(19) United States
(12) Patent Application Publication
(10) Pub. No.: US 2026/0359475 A1
(43) Pub. Date: Nov. 12, 2026

(54) Systems and Methods for Relational Database Management

(71) Applicant: Veeva Systems Inc., Pleasanton, CA (US)

(72) Inventors: Arno Sosna, Pleasanton, CA (US); Egor George Zhelezovskiy, Hermitage, PA (US); Brian Scates, Oakland, CA (US); Matthew William Taylor, San Diego, CA (US); Christine Bui, Alameda, CA (US)

(73) Assignee: Veeva Systems Inc., Pleasanton, CA (US)

(21) Appl. No.: 19/675,411

(22) Filed: May 12, 2026

Related U.S. Application Data
(60) Provisional application No. 63/804,478, filed on May 12, 2025.

(51) Int. Cl. G06F 21/62; G06F 16/29; G06F 40/174; G16H 40/20

(52) U.S. Cl. CPC G06F 21/6245; G06F 16/29; G06F 40/174; G16H 40/20

(57) ABSTRACT

A system comprises a first database storing patient data including protected health information or personally identifiable information associated with patients, and a second database separate from the first database storing customer relationship management data associated with healthcare providers. A server in communication with the first and second databases comprises processors and memory storing instructions that cause the server to receive, from a user computing device associated with a field representative, a request to access patient data associated with a patient case, retrieve from the first database patient data associated with the patient case, retrieve from the second database healthcare provider data associated with a healthcare provider linked to the patient case, and transmit to the user computing device a user interface displaying the patient data and the healthcare provider data while maintaining the patient data in the first database separate from the second database.

### CROSS-REFERENCE TO RELATED APPLICATIONS

This application claims priority to U.S. Provisional Application No. 63/804,478, titled “Systems and Methods for Patient CRM”, filed May 12, 2025, which is hereby incorporated by reference in its entirety.

### FIELD OF INVENTION

The present disclosure relates to database management systems, and more particularly to systems and methods for managing secure data and patient services programs within a segregated database environment that facilitates compliance with regulations governing protected health information and personally identifiable information.

### BACKGROUND

Enterprise customer relationship management (CRM) systems have become widely adopted across various industries to manage interactions with customers, track communications, and organize business processes. These systems typically store contact information, preferences, and interaction histories in centralized databases that can be accessed by multiple users within an organization.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a system is provided. The system comprises a first database configured to store patient data including at least one of protected health information or personally identifiable information associated with a plurality of patients. The system further comprises a second database separate from the first database, the second database configured to store customer relationship management data associated with a plurality of healthcare providers. The system further comprises a server in communication with the first database and the second database, the server comprising one or more processors and a memory storing instructions that, when executed by the one or more processors, cause the server to receive, from a user computing device associated with a field representative, a request to access patient data associated with a patient case. The instructions further cause the server to retrieve, from the first database, patient data associated with the patient case. The instructions further cause the server to retrieve, from the second database, healthcare provider data associated with a healthcare provider linked to the patient case. The instructions further cause the server to transmit, to the user computing device, a user interface displaying the patient data and the healthcare provider data while maintaining the patient data in the first database separate from the second database.

According to other aspects of the present disclosure, the system may include one or more of the following features. The instructions may further cause the server to receive a fax document containing patient information, perform optical character recognition on the fax document to extract text from the fax document, identify a plurality of data fields within the extracted text, and automatically populate a patient intake form with the plurality of data fields. The instructions may further cause the server to receive a selection of a field in the patient intake form and display a visual indication on the fax document corresponding to a location from which data populating the selected field was extracted. The user interface may include a visual indicator adjacent to fields containing at least one of protected health information or personally identifiable information. Data associated with fields having the visual indicator may be encrypted during transmission. The instructions may further cause the server to receive geographic location data associated with a plurality of patient cases, display a map interface depicting locations associated with the plurality of patient cases, receive a selection of a geographic area on the map interface, and identify patient cases located within the selected geographic area for scheduling. The instructions may further cause the server to generate a schedule grouping the identified patient cases based on proximity to each other. The patient data stored in the first database may not be stored locally on the user computing device. The patient data stored in the first database may not be stored in the second database. The instructions may further cause the server to track a patient journey comprising a plurality of workflow stages including at least one of intake, enrollment, benefits verification, coverage verification, fulfillment, or adherence monitoring.

According to another aspect of the present disclosure, a method is provided. The method comprises storing, in a first database, patient data including at least one of protected health information or personally identifiable information associated with a plurality of patients. The method further comprises storing, in a second database separate from the first database, customer relationship management data associated with a plurality of healthcare providers. The method further comprises receiving, by a server from a user computing device, a request to access data associated with a patient case. The method further comprises retrieving, by the server from the first database, patient data associated with the patient case. The method further comprises retrieving, by the server from the second database, healthcare provider data associated with a healthcare provider linked to the patient case. The method further comprises transmitting, by the server to the user computing device, a user interface displaying the patient data and the healthcare provider data while maintaining the patient data in the first database separate from the second database.

According to other aspects of the present disclosure, the method may include one or more of the following features. The method may further comprise receiving a fax document containing patient information, performing optical character recognition on the fax document to extract text from the fax document, identifying a plurality of data fields within the extracted text, and automatically populating a patient intake form with the plurality of data fields. The method may further comprise receiving a selection of a field in the patient intake form and displaying a visual indication on the fax document corresponding to a location from which data populating the selected field was extracted. The method may further comprise displaying a visual indicator adjacent to fields in the user interface containing at least one of protected health information or personally identifiable information. The method may further comprise receiving geographic location data associated with a plurality of patient cases, displaying a map interface depicting locations associated with the plurality of patient cases, receiving a selection of a geographic area on the map interface, and identifying patient cases located within the selected geographic area for scheduling. The method may further comprise tracking a patient journey comprising a plurality of workflow stages including at least one of intake, enrollment, benefits verification, coverage verification, fulfillment, or adherence monitoring.

According to another aspect of the present disclosure, a non-transitory computer-readable medium storing instructions is provided. The instructions, when executed by one or more processors, cause the one or more processors to maintain a first database storing patient data including at least one of protected health information or personally identifiable information associated with a plurality of patients. The instructions further cause the one or more processors to maintain a second database separate from the first database, the second database storing customer relationship management data associated with a plurality of healthcare providers. The instructions further cause the one or more processors to receive, from a user computing device associated with a field representative, a request to access data associated with a patient case. The instructions further cause the one or more processors to retrieve, from the first database, patient data associated with the patient case. The instructions further cause the one or more processors to retrieve, from the second database, healthcare provider data associated with a healthcare provider linked to the patient case. The instructions further cause the one or more processors to transmit, to the user computing device, a user interface displaying the patient data and the healthcare provider data while maintaining the patient data in the first database separate from the second database.

According to other aspects of the present disclosure, the non-transitory computer-readable medium may include one or more of the following features. The instructions may further cause the one or more processors to receive a fax document containing patient information, perform optical character recognition on the fax document to extract text from the fax document, and automatically populate a patient intake form with data extracted from the fax document. The instructions may further cause the one or more processors to receive a selection of a field in the patient intake form and display a visual indication on the fax document corresponding to a location from which data populating the selected field was extracted. The instructions may further cause the one or more processors to receive geographic location data associated with a plurality of patient cases, display a map interface depicting locations associated with the plurality of patient cases, and enable selection of patient cases within a geographic area for grouped scheduling.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 depicts a schematic diagram of a customer relationship management system, according to aspects of the present disclosure.

FIG. 2 depicts a schematic diagram of a controlled content repository and associated components, according to an embodiment.

FIG. 3 depicts a block diagram of a computing device, according to an embodiment.

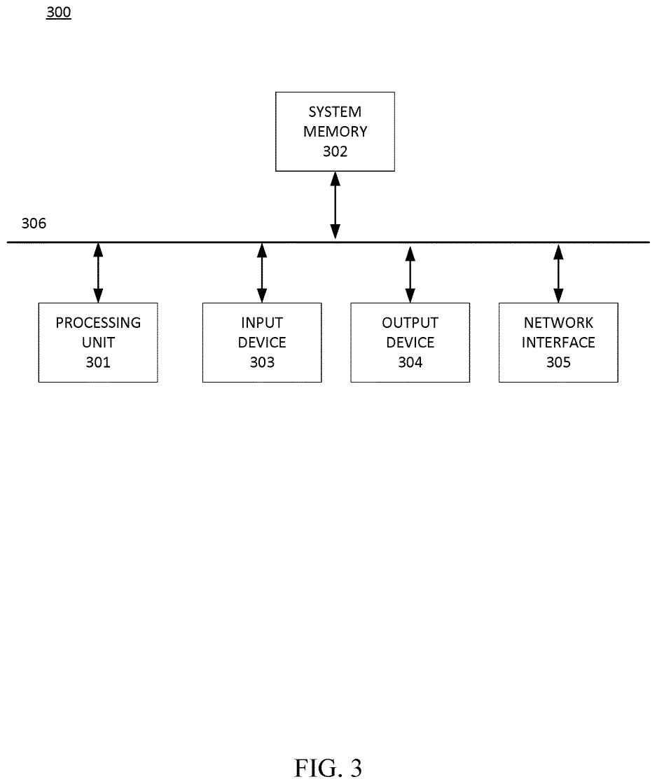

FIG. 4 depicts an exemplary user interface displaying a patient CRM dashboard, according to aspects of the present disclosure.

FIG. 5 depicts an exemplary user interface displaying a case inbox with patient case details, according to an embodiment.

FIG. 6 depicts an exemplary user interface displaying a fax inbox with a fax document, according to aspects of the present disclosure.

FIG. 7 depicts an exemplary user interface displaying a patient search and fax document with patient information, according to an embodiment.

FIG. 8 depicts an exemplary user interface displaying a patient intake form with data fields, according to aspects of the present disclosure.

FIG. 9 depicts an exemplary user interface displaying a patient intake form with auto-populated data, according to an embodiment.

FIG. 10 depicts an exemplary user interface displaying patient services and patient journey information, according to aspects of the present disclosure.

FIG. 11 depicts an exemplary user interface displaying patient intake with prescription and insurance information, according to an embodiment.

FIG. 12 depicts an exemplary user interface displaying a patient journey with workflow stages, according to aspects of the present disclosure.

FIG. 13 depicts an exemplary user interface displaying a case task with communication guidance, according to an embodiment.

FIG. 14 depicts an exemplary user interface displaying an active call interface, according to aspects of the present disclosure.

FIG. 15 depicts an exemplary user interface displaying a case comment entry interface, according to an embodiment.

FIG. 16 depicts an exemplary user interface displaying a patient journey with completed workflow stages, according to aspects of the present disclosure.

FIG. 17 depicts an exemplary user interface displaying a coverage verification workflow, according to an embodiment.

FIG. 18 depicts an exemplary user interface displaying a benefits verification task, according to aspects of the present disclosure.

FIG. 19 depicts an exemplary user interface displaying a coverage outcome entry interface, according to an embodiment.

FIG. 20 depicts an exemplary user interface displaying a patient journey with coverage verification details, according to aspects of the present disclosure.

FIG. 21 depicts an exemplary user interface displaying a benefits summary fax draft, according to an embodiment.

FIG. 22 depicts an exemplary user interface displaying a patient journey with fulfillment stage, according to aspects of the present disclosure.

FIG. 23 depicts an exemplary user interface displaying a patient journey with fulfillment and shipment confirmation, according to an embodiment.

FIG. 24 depicts an exemplary user interface displaying a patient journey with adherence monitoring, according to aspects of the present disclosure.

FIG. 25 depicts an exemplary user interface displaying a geographic scheduling map with patient cases, according to an embodiment.

FIG. 26 depicts an exemplary user interface displaying a field representative home dashboard, according to aspects of the present disclosure.

FIG. 27 depicts an exemplary user interface displaying a patient list with workflow status indicators, according to an embodiment.

FIG. 28 depicts an exemplary user interface displaying open and scheduled cases, according to aspects of the present disclosure.

FIG. 29 depicts an exemplary user interface displaying a geographic map with case details, according to an embodiment.

FIG. 30 depicts an exemplary user interface displaying a geographic map with case selection for scheduling, according to aspects of the present disclosure.

FIG. 31 depicts an exemplary user interface displaying a scheduling request interface, according to an embodiment.

FIG. 32 depicts an exemplary user interface displaying a field representative performance dashboard, according to aspects of the present disclosure.

FIG. 33 depicts an exemplary user interface displaying a weekly schedule calendar view, according to an embodiment.

### DETAILED DESCRIPTION

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, the subject technology is not limited to the specific details set forth herein and may be practiced without these specific details. In some instances, well-known structures and components are shown in block diagram form in order to avoid obscuring the concepts of the subject technology.

This disclosure arises from the realization that in the healthcare and life sciences industries, organizations face particular challenges when managing patient-related data. Healthcare providers, pharmaceutical companies, and patient services organizations handle sensitive information that is subject to regulatory requirements governing the protection of protected health information (PHI) and personally identifiable information (PII). These regulations impose restrictions on how such data may be stored, accessed, transmitted, and processed.

Traditional CRM systems designed for general commercial use may not adequately address the compliance considerations associated with patient data management. When patient information is commingled with other customer data in a shared database environment, organizations may encounter difficulties in implementing appropriate access controls, audit trails, and data handling procedures that satisfy regulatory requirements.

Additionally, patient services programs often involve complex workflows that span multiple stakeholders, including healthcare providers, field representatives, reimbursement specialists, and patient support personnel. Coordinating these workflows while maintaining appropriate data segregation and compliance controls can present technical and operational challenges.

The intake and enrollment processes for patient services programs frequently rely on legacy communication methods, such as fax transmissions, which may involve manual data entry and transcription. These manual processes can introduce delays and potential errors into patient onboarding workflows.

Furthermore, field personnel who interact with both healthcare providers and patient services systems may benefit from tools that facilitate coordination across these different domains while respecting the boundaries between patient data and other CRM data.

The present disclosure addresses these challenges through a segregated database architecture in which secure patient data including protected health information and personally identifiable information is stored in a first database that is separate from a second database storing healthcare provider customer relationship management data. This architectural separation facilitates compliance with regulations governing sensitive patient information by ensuring that patient data is not commingled with other CRM data and is not stored locally on user computing devices. The system enables field representatives and other authorized personnel to access data from both databases through a unified user interface while maintaining the underlying data segregation, thereby supporting coordination across patient services and healthcare provider interactions without compromising data isolation. To address inefficiencies associated with legacy intake processes, the system may employ optical character recognition and automated data extraction techniques to process fax documents and populate patient intake forms, reducing manual data entry and potential transcription errors. The system may further provide visual indicators that identify fields containing protected health information or personally identifiable information, with associated security handling including encryption during transmission. Additionally, geographic scheduling tools may assist field personnel in coordinating visits across multiple patient cases by enabling selection of cases within a geographic area for grouped scheduling, thereby improving operational efficiency for personnel who cover large territories.

The present disclosure describes systems, methods, and non-transitory computer-readable media for managing patient data and patient services programs within a customer relationship management (CRM) environment. In some aspects, a patient CRM system may be configured to manage protected health information (PHI) and personally identifiable information (PII) associated with patients while maintaining separation from healthcare provider CRM data. This segregated database architecture may facilitate compliance with regulations governing the handling of sensitive patient information.

In some cases, patient services programs involve complex workflows that span multiple stakeholders, including healthcare providers, patient support specialists, benefits specialists, field reimbursement managers, and other personnel. These workflows may include intake and enrollment processes, benefits verification, coverage verification, prior authorization handling, fulfillment coordination, and adherence monitoring. A patient CRM system as described herein may provide tools to manage these workflows while maintaining appropriate data segregation between patient data and healthcare provider data.

According to aspects of the present disclosure, a system may comprise a first database configured to store patient data including PHI or PII associated with a plurality of patients, and a second database separate from the first database configured to store customer relationship management data associated with a plurality of healthcare providers. A server in communication with the first database and the second database may retrieve data from both databases and present a unified user interface to users while maintaining the underlying data separation. In some implementations, patient data stored in the first database may not be stored in the second database, and patient data may not be stored locally on user computing devices.

According to other aspects of the present disclosure, a method may be performed for managing patient data within a segregated database environment. The method may comprise storing patient data in a first database, storing healthcare provider CRM data in a second database separate from the first database, and retrieving data from both databases in response to user requests while maintaining the separation between the databases.

According to further aspects of the present disclosure, a non-transitory computer-readable medium storing instructions may be provided. The instructions, when executed by one or more processors, may cause the one or more processors to maintain separate databases for patient data and healthcare provider data, process requests for data associated with patient cases, and transmit user interfaces displaying combined data while preserving the underlying database segregation.

In some implementations, the patient CRM system may support various user types, including call center personnel who perform patient intake and enrollment, benefits specialists who manage coverage verification workflows, and field representatives who coordinate with healthcare providers. The system may provide different views and access controls based on user roles while maintaining consistent data segregation practices across all user interactions.

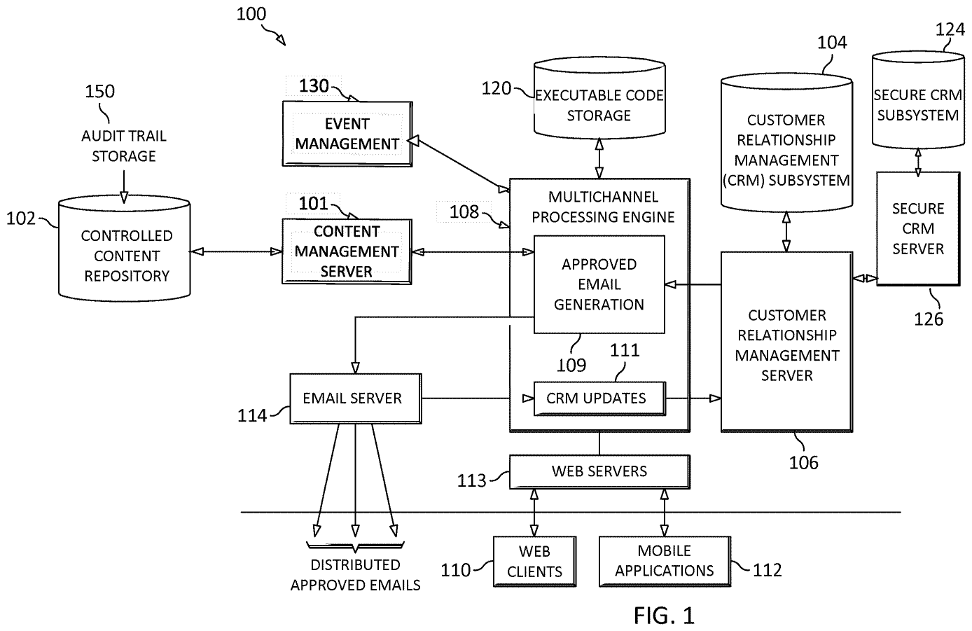

FIG. 1 is a system overview illustrating an embodiment of an secure patient CRM system 100. The presently disclosed embodiment comprises a controlled content repository 102, a Customer Relationship Management (CRM) server 106, and a multichannel processing engine 108, and an event management controller 130. The customer relationship management server 106 may provide access to a customer relationship management subsystem 104, and the multichannel processing engine 108 may be coupled to an email server 114, the content management server 101, the CRM server 106 and the event management controller 130. In one embodiment, the customer relationship management subsystem 104 and/or the email server 114 may be operated by a third party. The multichannel processing engine 108 may be accessed by users such as company sales representatives through web clients 110 or through mobile apps 112 (such as iOS, Android, Blackberry, or Windows Mobile systems), communicating with the multichannel processing engine 108 through web servers 113. Although the users may be described in the present application as being company sales representatives, this particular described embodiment is not intended to limit the generality of the claims that may eventually issue in patents stemming from the present disclosure.

The controlled content repository 102 is designed to have a process for developing approved content that is sharable across multiple users, such as shareholders, reviewers, managers, marketing personnel, sales representatives, etc. The content generated in the controlled content repository 102 may be accessed on a regulated basis and used to generate approved electronic communications. This regulated basis may be determined, in part, by the company as a whole and additionally by interaction with data from the customer relationship management subsystem 104, described in further detail below. In one embodiment, approved content, customer profile information, customer preferences, and regulatory limitations and requirements may be stored in a table in the controlled content repository 102. In addition to storage and development of content, the controlled content repository 102 may also store an audit trail, tracking exact content of communications as they were sent by the user (e.g. pharmaceutical rep), as well as metadata about the communications and information regarding the content accessed by recipients (e.g. HCP).

The customer relationship management subsystem 104 contains all professional information of HCPs that may be available to users, including contact information, licensing information, areas of practice, and specialties. In addition, the customer relationship management subsystem 104 may also be capable of storing configurations regarding specific preferences, regulatory limitations and requirements, and other fields that will facilitate the generation of appropriate approved electronic communications, in general or on a by recipient basis. These preferences and/or requirements include both the preferences of the user (e.g., maintaining account lists) as well as the preferences of the enterprise (e.g., employers of the users), discussed in further detail below. In some examples, the approved content and email templates may be pre-processed and stored in the controlled content repository 102 and provided to the multichannel processing engine 108 during the process for generating a tailored approved email. In other examples, the customer relationship management subsystem 104 may have a content management subsystem and may provide the approved content and the templates.

In other examples, the customer relationship management subsystem 104 may have a content management subsystem and may provide the approved content. The customer relationship management subsystem 104 may store event related data, e.g., a link to approved content in the controlled content repository 102, a speaker list, upcoming and past event date/time/locations/topics, content associated with each event, content sharing rules allowing access to content based on speaker qualifications, contracts associated with an associated speaker, and webinar tracking information as will be described in detail below.

In one embodiment, the event related data, e.g., a link to approved content in the controlled content repository 102, a speaker list, upcoming and past event date/time/locations/topics, content associated with each event, content sharing rules allowing access to content based on speaker qualifications, contracts associated with an associated speaker, and webinar tracking information may be stored in the controlled content repository 102.

In this embodiment, the customer relationship management subsystem 104 is capable of communication with multiple sources through the customer relationship management server 106 or through other channels to maintain a current and accurate collection of information regarding customer accounts. The interface with the multiple sources can be, for example, through an Applications Programming Interface or API, as the API interface will allow compatibility with a flexible array of third-party provider servers. The information being updated may include, but is not limited to, licensing information, area of practice, and location of the various customer contacts. In this manner, the customer relationship management subsystem 104 pulls the approved version of what represents an account or physician, which then pulls from multiple networks to ensure that the information regarding an account is up-to-date. The customer relationship management subsystem 104 may also be used to determine the type of domain an email communication is delivered through. A recipient in Spain may receive an email from “Companyx.es, whereas a recipient in Germany would receive the same email from “Companyx.ge.” This may allow for additional branding options for the company controlling and sending the electronic communications.

With further reference to the customer relationship management subsystem 104, this system may be a cloud-based customer database that provides a central access to store and distribute consistent data across customer companies as well as their possible third-party partners and agencies that are used to keep this data updated. This system can provide standard data formats and provide an easy and automated way for customers (e.g. pharmaceutical companies campaign manager) and/or users (e.g. pharmaceutical reps) to have access to coordinated and frequently updated CRM data and to use that coordinated data for sending approved electronic communications in accordance with the system described herein. In an embodiment, the multichannel processing engine 108 is responsible for combining the customer account information from the customer relationship management subsystem 104 with content available from the controlled content repository 102. Within the customer relationship management subsystem 104, customer accounts may be assigned a set of alignment rules which determine specific pieces of content that are available for use from the controlled content repository 102. The multichannel processing engine 108 may apply these rules and supply the user with a list of approved tailored email templates and pieces of content that may then be used to construct a tailored approved email communication. Tailored approved email generation occurs within the multichannel processing engine 108 according to executable code computer instructions stored in executable code storage 120. The executable code comprises computer readable instructions stored on the computer readable medium (the code storage medium 120). The executable code storage 120 is in communication with the various computing machines in the system 100 such as the customer relationship management server 106 and the multichannel processing engine 108. The same or another executable code storage 120 may be accessed by the previously described components of the controlled content repository 102 for providing separate computer readable code for operating upon by processing machines in that system. In all cases, the code is programmed to perform the functions that are described in the present embodiments and/or additional functions according to system design needs.

Communication between the multichannel processing engine 108 and the customer relationship management subsystem 104 may occur via the customer relationship management server 106, which acts as an interface between the two. The customer relationship management server 106 may act solely as an entry and exit point for the customer relationship management subsystem 104. The user may access the multi-channel processing engine 108 through either a Web Client 110 or through the mobile apps 112 (such as iOS, Android, Blackberry, or Windows Mobile systems).

The event management controller 130 may store event related information, such as information about attendees and speakers, event content, and event details.

Secure patient CRM system 100 includes a secure CRM subsystem 124, configured to store patient data including protected health information (PHI) and personally identifiable information (PII) associated with a plurality of patients. Secure CRM subsystem 124 may be separate from and operate independently of the customer relationship management subsystem 104, which is configured to store customer relationship management data associated with a plurality of healthcare providers (HCPs).

The secure CRM subsystem 124 may store patient-specific data including, but not limited to, patient demographic information, contact information, medical history, prescription information, insurance details, enrollment status, benefits verification data, coverage determination outcomes, prior authorization records, fulfillment tracking information, and adherence monitoring data. In some implementations, the secure CRM subsystem 124 may store data associated with patient cases, where each patient case represents a patient's participation in a patient services program and tracks the patient's journey through various workflow stages.

The customer relationship management subsystem 104 may store healthcare provider data including, but not limited to, HCP contact information, licensing information, areas of practice, specialties, account preferences, alignment rules, and interaction histories. The customer relationship management subsystem 104 may also store data associated with pharmaceutical representatives, field reimbursement managers, and other personnel who interact with healthcare providers.

In some aspects, the secure CRM subsystem 124 and the customer relationship management subsystem 104 may be implemented as physically separate database instances, logically separate database partitions, or separate database schemas within a database management system. The separation between the secure CRM subsystem 124 and the customer relationship management subsystem 104 may be enforced at the database level, the application level, or both, to ensure that patient data stored in the secure CRM subsystem 124 is not commingled with healthcare provider data stored in the customer relationship management subsystem 104.

In some implementations, the secure CRM subsystem 124 may be subject to enhanced security controls relative to the customer relationship management subsystem 104. For example, data stored in the secure CRM subsystem 124 may be encrypted at rest using encryption algorithms compliant with applicable regulatory requirements. Access to the secure CRM subsystem 124 may be controlled through security profiles and permission sets that restrict which users may access patient data based on user roles and responsibilities. In some cases, fields containing PHI or PII within the secure CRM subsystem 124 may be masked in audit trails and administrative interfaces to prevent unauthorized exposure of sensitive information.

In some aspects, the first database and the second database may be linked through one or more reference identifiers that enable cross-database queries without commingling patient data with healthcare provider data. For example, a patient case record stored in the secure CRM subsystem 124 may include a healthcare provider identifier that references a corresponding healthcare provider record in the customer relationship management subsystem 104. The healthcare provider identifier may be a unique alphanumeric code, a national provider identifier (NPI), or another identifier that allows the server to retrieve associated healthcare provider data from the customer relationship management subsystem 104 when processing a request related to a patient case.

In some implementations, the mapping between patient cases and healthcare providers may be established during the patient intake process. When a patient enrollment form is received, the system may associate the patient case with one or more healthcare providers based on information provided in the enrollment form, such as the prescribing physician or the healthcare facility where the patient received a diagnosis. The association may be stored as a reference identifier in the patient case record within the secure CRM subsystem 124, while the detailed healthcare provider information remains stored exclusively in the customer relationship management subsystem 104.

In some aspects, the server may perform cross-database retrieval at query time rather than pre-populating patient records with healthcare provider data. When a request is received from a user computing device, the server may first retrieve the patient case data from the secure CRM subsystem 124, extract the healthcare provider reference identifier from the patient case data, and then use the reference identifier to query the customer relationship management subsystem 104 for the associated healthcare provider data. The server may then combine the retrieved data for presentation in the user interface without persistently storing the combined data in either database.

In some implementations, patient data stored in the secure CRM subsystem 124 may not be stored locally on user computing devices. When a field representative or other user accesses patient data through a client application, the patient data may be retrieved from the secure CRM subsystem 124 and displayed in the user interface without being cached or persisted on the user computing device. This approach may reduce the risk of unauthorized access to patient data in the event that a user computing device is lost, stolen, or compromised.

In some aspects, patient data stored in the secure CRM subsystem 124 may not be replicated to or stored in the customer relationship management subsystem 104. The segregation between the two databases may be maintained throughout all system operations, including data retrieval, data updates, reporting, and analytics. This architectural separation may facilitate compliance with regulations governing the handling of PHI and PII by ensuring that patient data remains isolated within the secure CRM subsystem 124.

In some implementations, the system may support multiple healthcare provider associations for a single patient case. For example, a patient case may be associated with a prescribing physician, a treating facility, and one or more specialists involved in the patient's care. Each association may be represented by a separate reference identifier stored in the patient case record, allowing the server to retrieve data for multiple healthcare providers when generating the user interface.

In some aspects, the mapping between patient cases and healthcare providers may be updated as the patient journey progresses. For example, if a patient is referred to a different healthcare provider or transfers care to a new facility, the system may update the reference identifiers in the patient case record to reflect the new associations while maintaining the historical associations for audit and tracking purposes.

The secure patient CRM system may include a server (e.g., secure CRM server 126) in communication with the first database and the second database. The server may comprise one or more processors and a memory storing instructions that, when executed by the one or more processors, cause the server to perform various data management and processing operations. In some implementations, secure CRM server 126 may be implemented as a customer relationship management server that provides access to both the secure CRM subsystem storing patient data and the customer relationship management subsystem storing healthcare provider data.

In some aspects, the server may include a processing unit configured to execute computer instructions stored in a computer-readable medium. The processing unit may be a central processing unit (CPU) or another type of processor. The memory of the server may include computer storage media in the form of volatile and nonvolatile memory such as read-only memory (ROM) and random-access memory (RAM). The memory may store instructions and data including operating system components, program modules, and application programs that support data retrieval and processing operations.

The server may be in communication with the first database and the second database through network connections. In some cases, the server may communicate with the first database to retrieve patient data including protected health information or personally identifiable information, and may communicate with the second database to retrieve healthcare provider customer relationship management data. As described above, patient data stored in the first database may not be stored in the second database, and the server may maintain the separation between the first database and the second database during data retrieval and processing operations.

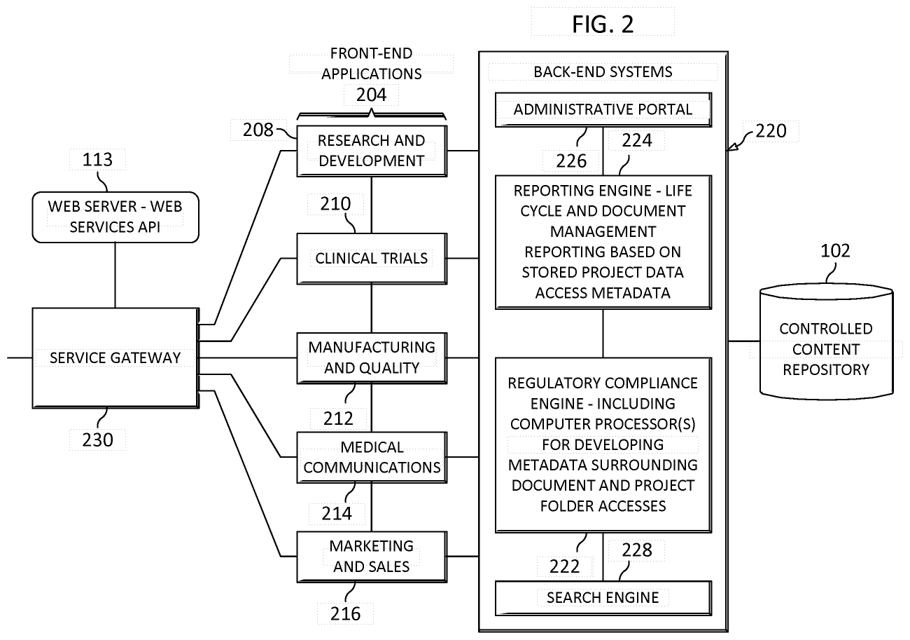

FIG. 2 provides a description of the controlled content repository 102 with additional specific applications and interfaces connected thereto. In an embodiment, this controlled content repository 102 is a cloud-based or distributed network-based system for consolidating an enterprise's data, often times integrating multiple content repositories in an enterprise into a single system having coordinated control, measuring, and auditing of data creation, access and distribution.

In an embodiment of the controlled content repository 102 for the life sciences industry, as illustrated in the figure, this repository 102 can include specific data collections for the following areas and/or business process-specific front-end applications 204: The Research & Development (R&D) front end application 208 provides for an aggregation of materials in support of research and initial clinical trial submissions through building organized and controlled content repositories within the controlled content repository 102. Elements that can be stored, organized, and managed through this frontend include submission bills of materials, Drug Information Association (DIA) reference models support, and submission-ready renderings. This front end 208 is designed to provide an interface to the controlled content repository 102 whereby researchers, contract research organizations (CROs), and other collaboration partners can access and/or distribute content through a single controlled document system.

The clinical trials front-end application 210 provides for faster and more organized access to trial documents and reports, while supporting seamless collaboration between sponsors, CROS, sites, investigators and other trial participants. Specific features both ease study and site administration as well as support the DIA trial master file (TMF) reference model. Having this front-end application providing access to the controlled content repository 102 further provides for efficient passing off of controlled content repository content between this phase and other phases of the life sciences development process. The manufacturing and quality application 212 enables the creation, review, approval and distribution of controlled documents across the organization and with external partners in the context of materials control and other manufacturing elements. The application 212 provides functionality in support of the manufacturing process including watermarking, controlled print, signature manifestation and “Read and Understood” signature capabilities. The documents and metadata associated with this process is managed and stored in the controlled content repository 102 whereby it can be assured that the related documents are not distributed in contravention of law and company policy.

The medical communications application 214 provides for communications with medical facilities, including call center access, integration, and interface functionality. Particular access control features and metadata associated with this application 214 include expiration and periodic review elements, multi-channel support, global documents and automatic response package generation through the controlled content repository 102.

The marketing and sales application 216 application provides an end-to-end solution for the development, approval, distribution, expiration and withdrawal of promotional mate rials. Specific features include Support for global pieces, approved Form FDA 2253 (or similar international forms) form generation, online document, and video annotation, and a built-in digital asset library (DAL). Again, the communications may be through the controlled content repository 102. In disclosed embodiments, there are provided a number of back-end system applications 220 that provide for the management of the data, forms, and other communications in the controlled content repository. For example, the back-end systems applications 220 may include a regulatory compliance engine 222 to facilitate regulatory compliance, including audit trail systems, electronic signatures systems, and system traceability to comply with government regulations, such as 21 CFR Part 11, Annex 11 and GXP-related requirements. The regulatory compliance engine 222 may include processors for developing metadata surrounding document and project folder accesses so from a regulatory compliance standpoint it can be assured that only allowed accesses have been permitted. The regulatory compliance engine 122 may further includes prevalidation functionality to build controlled content in support of installation qualification (IQ) and/or operational qualification (OQ), resulting in significant savings to customers for their system validation costs.

In further disclosed embodiments, the back-end systems 220 may contain a reporting engine 224 that reports on documents, their properties and the complete audit trail of changes. These simple-to-navigate reports show end users and management how content moves through its life cycle overtime, enabling the ability to track plan versus actual and identify process bottlenecks. The reporting engine may include processors for developing and reporting life cycle and document management reporting based on Stored project data and access metadata relative to documents, forms and other communications stored in the controlled content repository 102.

In further disclosed embodiments, the back-end systems 220 can include an administrative portal 226 whereby administrators can control documents, properties, users, security, workflow and reporting with a simple, point-and-click web interface. Customers also have the ability to quickly change and extend the applications or create brand new applications, including without writing additional software code.

In further disclosed embodiments, the back-end systems 220 may include a search engine 228 whereby the controlled content repository 102 can deliver simple, relevant and secure searching. In providing this holistic combination of front-end applications 204 and back-end system applications 220, the various applications can further be coordinated and communicated with by the service gateway 230, which in turn can provide for communications with various web servers and/or web services APIs 113. Such web servers and/or web services APIs 113 can include access to the content and metadata layers of some or all of the various applications 204 and systems 220, enabling seamless integration among complementary systems.

In the context of the described embodiments, the particular features and capabilities integrate with the other elements of the controlled email communication system 100 such that particular features are usable within the various user screens. For example, and without limitation to any other possible combination of features, in the below-described description of generating a tailored approved electronic communication, the above-described search portal can be used to search for approved attachments (i.e. content) within the controlled content repository 102 which may be then attached to one of the tailored template emails being prepared for sending to a recipient. And the synergy of combining these particular systems is that the generation of the controlled content can be done in a controlled fashion such that the approved content can be sent to recipients with greater confidence in the integrity of the data being sent.

Secure patient CRM 100 may include a first database (e.g., secure CRM subsystem 124) configured to store secure patient data including at least one of protected health information (PHI) or personally identifiable information (PII) associated with a plurality of patients. The first database may be referred to as a patient CRM database and may operate separately from other databases in the system to maintain regulatory compliance and safeguard sensitive patient information.

In some implementations, the first database may store patient data using a patient-centric data model. The patient-centric data model may organize data around individual patient records, where each patient record may include demographic information, medical information, and program-related information. Demographic information may include patient name, date of birth, contact information such as phone number and email address, physical address, and gender. Medical information stored in the first database may include prescription details, treatment information, and medical history. Program-related information may include insurance details, consent records, and patient program enrollment data.

In some cases, the first database may store patient data in association with patient cases. A patient case may represent an instance of a patient's participation in a patient services program and may link patient data to associated workflow information. The first database may store case-related data including case identifiers, case status, case ownership assignments, and case notes.

The first database may maintain patient data separately from other CRM data stored in the system. In some aspects, patient data stored in the first database may not be stored in a second database that stores healthcare provider customer relationship management data. This separation may facilitate compliance with regulations governing the handling of protected health information and personally identifiable information by ensuring that sensitive patient data is not commingled with other types of CRM data.

In some implementations, the first database may apply security measures to fields containing protected health information or personally identifiable information. Fields designated as containing PHI or PII may be encrypted at rest within the first database. The first database may also mask PHI and PII values in audit trails such that audit history records indicate that a change occurred without revealing the actual values that were changed.

Access to patient data stored in the first database may be controlled through security profiles and permission sets. Based on a user's configured user type, the user may be granted access to objects containing patient data and to specific patient records. In some cases, call center users or users performing patient intake may have access to patient information across multiple records to enable cross-referencing based on patient identifiers such as name or phone number.

In some aspects, patient data stored in the first database may not be stored locally on user computing devices. When a user computing device requests access to patient data, the patient data may be retrieved from the first database and transmitted to the user computing device for display without being persisted in local storage on the user computing device. This approach may help maintain the segregation of patient data within the first database.

The first database may be implemented using cloud-based infrastructure. In some implementations, the first database may be part of a multi-tenant system where various elements of hardware and software may be shared by multiple customers. The first database may include computer storage media in the form of volatile and nonvolatile memory such as read-only memory (ROM) and random-access memory (RAM). The first database may store instructions and data including operating system components, program modules, and application programs that support patient data management operations.

The secure patient CRM system 100 may include a second database (e.g., CRM subsystem 104) separate from the first database. The second database may be configured to store customer relationship management data associated with a plurality of healthcare providers. As described above, the first database stores patient data including protected health information or personally identifiable information, and the second database maintains separation from the first database such that patient data stored in the first database is not stored in the second database.

In some implementations, the second database may store professional information associated with healthcare providers. Healthcare providers may include healthcare professionals (HCPs) such as physicians, as well as healthcare organizations such as hospitals and medical practices. The second database may store contact information for healthcare provider accounts, where contact information may include addresses, phone numbers, email addresses, and other communication details.

The second database may store licensing information associated with healthcare providers. Licensing information may include medical license numbers, license expiration dates, license status, and jurisdictions in which healthcare providers are licensed to practice. The second database may also store areas of practice associated with healthcare providers, where areas of practice may indicate medical disciplines or treatment areas in which healthcare providers focus their professional activities. In some cases, the second database may store specialty information indicating medical specialties or subspecialties held by healthcare providers.

In some aspects, the second database may store interaction histories associated with healthcare provider accounts. Interaction histories may include records of communications between users of the system and healthcare providers, including dates and times of interactions, communication channels used, topics discussed, and outcomes of interactions. The second database may track interactions conducted by sales representatives, field reimbursement managers, and other personnel who engage with healthcare providers.

The second database may store configurations regarding specific preferences associated with healthcare provider accounts. Preferences may include communication preferences indicating how healthcare providers prefer to receive communications, scheduling preferences, and content preferences. The second database may also store regulatory limitations and requirements associated with healthcare provider accounts, where regulatory limitations may govern what types of communications or content may be shared with particular healthcare providers.

In some implementations, the second database may store sales data and other fields that facilitate communications with healthcare providers. Sales data may include information about products discussed with healthcare providers, samples distributed, and promotional materials shared. The second database may support communications on a by-recipient basis, where communications may be tailored based on data stored in association with individual healthcare provider accounts.

The second database may communicate with multiple sources to maintain a current and accurate collection of information regarding healthcare provider accounts. Healthcare provider accounts may include group accounts representing healthcare organizations and individual accounts representing individual healthcare providers. The second database may interface with multiple sources through an Application Programming Interface (API), where the API interface allows compatibility with a flexible array of third-party provider servers. Information updated through these interfaces may include licensing information, area of practice, and location of healthcare provider accounts. In this manner, the second database may pull approved versions of account information and pull from multiple networks to ensure that information regarding healthcare provider accounts is up-to-date.

In some cases, the second database may be implemented as a cloud-based customer database that provides central access to store and distribute consistent data across customer companies as well as third-party partners and agencies. The second database may provide standard data formats and provide an automated way for users to have access to coordinated and frequently updated CRM data. In some implementations, the second database may be part of a multi-tenant system where various elements of hardware and software may be shared by multiple customers. In a multi-tenant system, a user may be associated with a particular customer, such as a pharmaceutical company that is a tenant of the system.

In some aspects, the second database may be operated by a third party. The second database may store event-related data including speaker lists, event dates, times, locations, and topics, content associated with events, content sharing rules, and contracts associated with speakers. Customer accounts stored in the second database may be assigned alignment rules that determine specific pieces of content available for use from a controlled content repository.

The separation between the first database and the second database may facilitate compliance with regulations governing protected health information and personally identifiable information. By maintaining patient data in the first database separate from healthcare provider customer relationship management data in the second database, the system may ensure that sensitive patient information is not commingled with other types of CRM data. Users such as field reimbursement managers may have access to data from both the first database and the second database to coordinate patient services and healthcare provider interactions, while the underlying data segregation is maintained.

In some aspects, secure CRM server 126 may access data associated with a patient case. In response to the request, secure CRM server 126 may retrieve, from secure CRM subsystem 124, patient data associated with the patient case. The retrieval operation may involve querying secure CRM subsystem 124 using a case identifier, a patient identifier, or other identifying information included in the request.

In some implementations, secure CRM server 126 may retrieve patient data based on a case identifier provided in the request. The case identifier may be a unique alphanumeric code assigned to a patient case when the patient case is created in the first database. Secure CRM server 126 may use the case identifier to locate the corresponding patient case record in the first database and retrieve associated patient data including demographic information, medical information, and program-related information linked to the patient case.

In some cases, secure CRM server 126 may retrieve patient data based on a patient identifier. The patient identifier may include a patient name, a phone number, a date of birth, or a combination of identifying attributes. When a user computing device submits a request containing patient identifying information, the server may query the first database to locate patient records matching the provided identifying information. Secure CRM server 126 may return matching patient records to the user computing device for selection when multiple records match the provided identifying information.

In some aspects, the retrieval of patient data from the first database may be subject to security controls. As described above, access to patient data stored in the first database may be controlled through security profiles and permission sets. When secure CRM server 126 receives a request to access patient data, secure CRM server 126 may verify that the user associated with the user computing device has authorization to access the requested patient data. The verification may include determining whether the user has access to objects containing patient data and whether the user has access to the specific patient records identified in the request.

In some implementations, secure CRM server 126 may apply record-level access controls when retrieving patient data from the first database. Record-level access controls may restrict which patient records a particular user may access based on the user's role, territory assignment, or other access criteria. For example, a field reimbursement manager may have access to patient cases assigned to the field reimbursement manager's territory, while a call center user performing patient intake may have access to patient information across multiple records to enable cross-referencing based on patient identifiers.

In some cases, secure CRM server 126 may retrieve patient data from the first database without storing the patient data locally on the user computing device. When the user computing device requests access to patient data, the server may retrieve the patient data from the first database and transmit the patient data to the user computing device for display. The patient data may be rendered in a user interface on the user computing device without being persisted in local storage on the user computing device. When the user navigates away from a view displaying patient data, the patient data may not remain stored on the user computing device.

In some aspects, secure CRM server 126 may retrieve patient data from the first database in real-time in response to user navigation within a client application. When a user navigates to a patients tab or accesses a specific patient record within the client application, secure CRM server 126 may retrieve the corresponding patient data from the first database and transmit the patient data to the user computing device for display. The real-time retrieval approach may ensure that users view current patient data from the first database rather than cached or stale data.

In some implementations, secure CRM server 126 may retrieve patient data associated with a patient case including case-related data. Case-related data may include case identifiers, case status, case ownership assignments, case severity, case type, case queue assignments, and case notes. Secure CRM server 126 may retrieve case-related data along with patient demographic and medical information to provide a comprehensive view of the patient case to the user.

In some cases, secure CRM server 126 may retrieve patient data from the first database in response to requests from different types of users. Users may include call center personnel who perform patient intake and enrollment, benefits specialists who manage coverage verification workflows, patient support specialists who coordinate patient communications, and field reimbursement managers who coordinate with healthcare providers. Secure CRM server 126 may retrieve patient data from the first database based on the access permissions associated with each user type while maintaining the segregation of patient data within the first database.

In some aspects, secure CRM server 126 may retrieve, from the second database, healthcare provider data associated with a healthcare provider linked to the patient case. As described above, patient cases stored in the first database may be linked to healthcare providers through reference identifiers such as healthcare provider identifiers or national provider identifiers (NPIs). When secure CRM server 126 receives a request to access data associated with a patient case, secure CRM server 126 may extract the healthcare provider reference identifier from the patient case record and use the reference identifier to query the second database for associated healthcare provider data.

In some implementations, secure CRM server 126 may retrieve healthcare provider data from the second database based on a healthcare provider identifier stored in the patient case record. The healthcare provider identifier may be established during the patient intake process when a patient enrollment form is received and processed. The enrollment form may include information identifying the prescribing physician, the treating facility, or other healthcare providers involved in the patient's care. Secure CRM server 126 may use the healthcare provider identifier to locate the corresponding healthcare provider record in the second database and retrieve associated healthcare provider data.

In some cases, the healthcare provider data retrieved from the second database may include contact information associated with the healthcare provider. Contact information may include office phone numbers, office fax numbers, email addresses, and physical addresses. Secure CRM server 126 may retrieve healthcare provider contact information to enable users to communicate with healthcare providers regarding patient cases.

In some aspects, secure CRM server 126 may retrieve licensing information associated with the healthcare provider from the second database. Licensing information may include medical license numbers, license status, and jurisdictions in which the healthcare provider is licensed to practice. Secure CRM server 126 may also retrieve areas of practice and specialty information associated with the healthcare provider.

In some implementations, secure CRM server 126 may retrieve healthcare provider data from the second database for multiple healthcare providers linked to a single patient case. A patient case may be associated with multiple healthcare providers, such as a prescribing physician and a treating facility. Each association may be represented by a separate reference identifier stored in the patient case record. Secure CRM server 126 may use each reference identifier to query the second database and retrieve data for each associated healthcare provider.

In some cases, secure CRM server 126 may retrieve healthcare provider data from the second database at query time rather than pre-populating patient records with healthcare provider data. When a request is received from a user computing device, secure CRM server 126 may first retrieve the patient case data from the first database, extract the healthcare provider reference identifiers from the patient case data, and then use the reference identifiers to query the second database for the associated healthcare provider data. Secure CRM server 126 may combine the retrieved data for presentation in the user interface without persistently storing the combined data in either database.

In some aspects, secure CRM server 126 may retrieve healthcare provider data from the second database in response to requests from field reimbursement managers. Field reimbursement managers may coordinate with healthcare providers to resolve issues that arise during the patient journey. When a field reimbursement manager accesses a patient case, secure CRM server 126 may retrieve healthcare provider data from the second database to enable the field reimbursement manager to contact the healthcare provider associated with the patient case. The field reimbursement manager may have access to data from both the first database and the second database to coordinate patient services and healthcare provider interactions while the underlying data segregation is maintained.

In some implementations, secure CRM server 126 may retrieve healthcare provider data from the second database to support scheduling operations. When a field reimbursement manager selects patient cases for grouped scheduling, secure CRM server 126 may retrieve healthcare provider data associated with each selected patient case. Secure CRM server 126 may use the retrieved healthcare provider data to identify sales representatives aligned with the healthcare providers associated with the selected patient cases. Scheduling requests may be transmitted to the identified sales representatives to coordinate joint visits between field reimbursement managers and sales representatives.

In some cases, secure CRM server 126 may retrieve updated healthcare provider data from the second database. As described above, the second database may communicate with multiple sources to maintain current information regarding healthcare provider accounts. When secure CRM server 126 retrieves healthcare provider data in response to a request, secure CRM server 126 may retrieve the current healthcare provider data from the second database, which may reflect updates received from external sources through API interfaces.

In some aspects, the server may transmit, to the user computing device, a user interface displaying the patient data and the healthcare provider data while maintaining the patient data in the first database separate from the second database. As described above, the server may retrieve patient data from the first database and healthcare provider data from the second database in response to a request from the user computing device. The server may combine the retrieved patient data and healthcare provider data for presentation in the user interface without persistently storing the combined data in either database.

In some implementations, the user interface may display patient data and healthcare provider data in a unified view that enables users to access information from both databases through a single interface. The user interface may include patient demographic information, medical information, and program-related information retrieved from the first database, as well as healthcare provider contact information, licensing information, and specialty information retrieved from the second database. The server may generate the user interface by assembling data elements from both databases and transmitting the assembled user interface to the user computing device for rendering.

In some cases, the user interface may include a patient overview screen that displays patient data retrieved from the first database. The patient overview screen may provide access to patient demographic information, prescription details, insurance details, and patient journey information. The user interface may also display healthcare provider information associated with the patient case, where the healthcare provider information is retrieved from the second database based on reference identifiers stored in the patient case record.

In some aspects, the server may transmit the user interface to the user computing device using network communication protocols. The server may format the user interface data for transmission and transmit the formatted data to the user computing device through a network interface. The user computing device may receive the transmitted data and render the user interface for display to the user.

In some implementations, the patient data stored in the first database may not be stored locally on the user computing device. When the server transmits the user interface to the user computing device, the patient data may be rendered for display without being persisted in local storage on the user computing device. The user computing device may display the patient data in the user interface while the patient data remains stored in the first database. When the user navigates away from a view displaying patient data, the patient data may not remain stored on the user computing device.

In some cases, the server may retrieve patient data from the first database in real-time in response to user navigation within a client application running on the user computing device. When a user navigates to a patients tab or accesses a patient case within the client application, the server may retrieve the corresponding patient data from the first database and transmit the patient data to the user computing device for display. The real-time retrieval approach may ensure that users view current patient data from the first database rather than cached data stored locally on the user computing device.

In some aspects, the user interface may display patient data and healthcare provider data in separate regions or panels while presenting a unified view to the user. The server may organize the user interface such that patient data retrieved from the first database is displayed in designated areas of the user interface, and healthcare provider data retrieved from the second database is displayed in other designated areas. The separation of display regions may correspond to the underlying separation between the first database and the second database.

In some implementations, the server may transmit the user interface to the user computing device in response to requests from different types of users. Users may include call center personnel, benefits specialists, patient support specialists, and field reimbursement managers. The server may generate user interfaces tailored to the access permissions and role-based requirements of each user type while maintaining the patient data in the first database separate from the second database.

In some cases, the server may transmit updated user interfaces to the user computing device as patient data or healthcare provider data changes. When data in the first database or the second database is updated, the server may retrieve the updated data and transmit an updated user interface to the user computing device. The user computing device may display the updated user interface without storing the patient data locally, thereby maintaining the segregation of patient data within the first database.

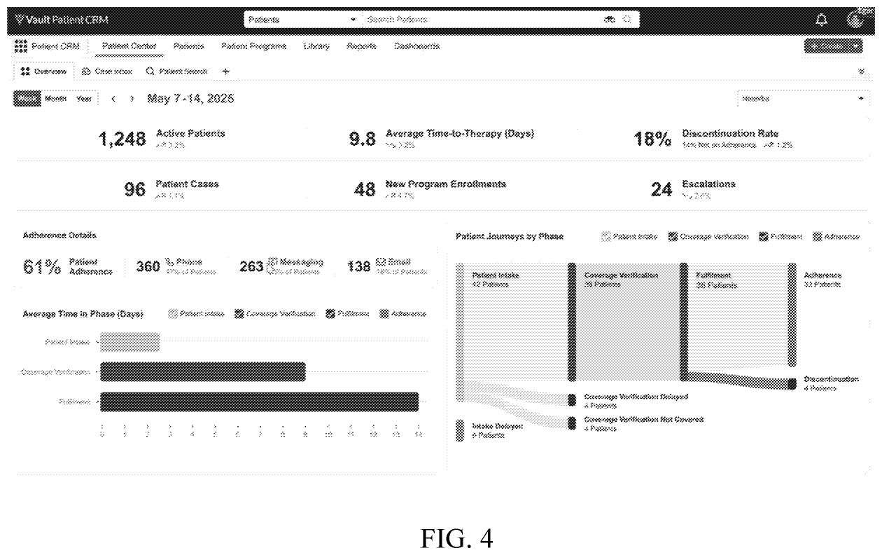

FIG. 4 depicts an exemplary user interface displaying a patient CRM dashboard, according to aspects of the present disclosure. The dashboard displays a case inbox with multiple inbox views including a My Cases view and channel-specific inboxes such as a fax inbox and an email inbox. The case list displays case identifiers (e.g., CA0000107, CA0000110, CA0006040), status indicators (Open, Normal, Escalated), patient names (e.g., Nicole Winston, Megan Mazza, Lane Marks), product associations (e.g., Natevba, CholeCap), case types (Coverage Verification, Task, Question), and a case activity feed showing case reassignments, comments, and timestamps. The interface enables users to view and manage patient cases from a centralized dashboard.

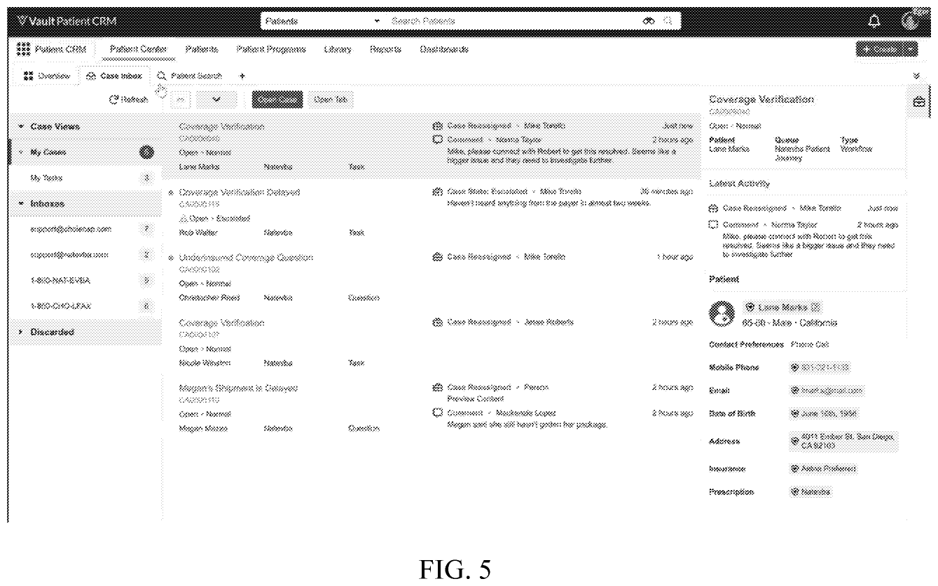

FIG. 5 depicts an exemplary user interface displaying a case inbox with patient case details, according to an embodiment. The interface displays a selected case with a case details panel showing a case identifier (CA0006040), status (Open, Normal), and patient information including contact preferences, mobile phone number (831-221-1133), email address (lmarks@mail.com), date of birth (Jun. 10, 1956), address (4011 Ember St. San Diego, CA 92103), insurance information (Aetna Preferred), and prescription details (Natevba). The interface displays the patient's current workflow stage (Coverage Verification) and queue assignment (Natevba Patient Journey).

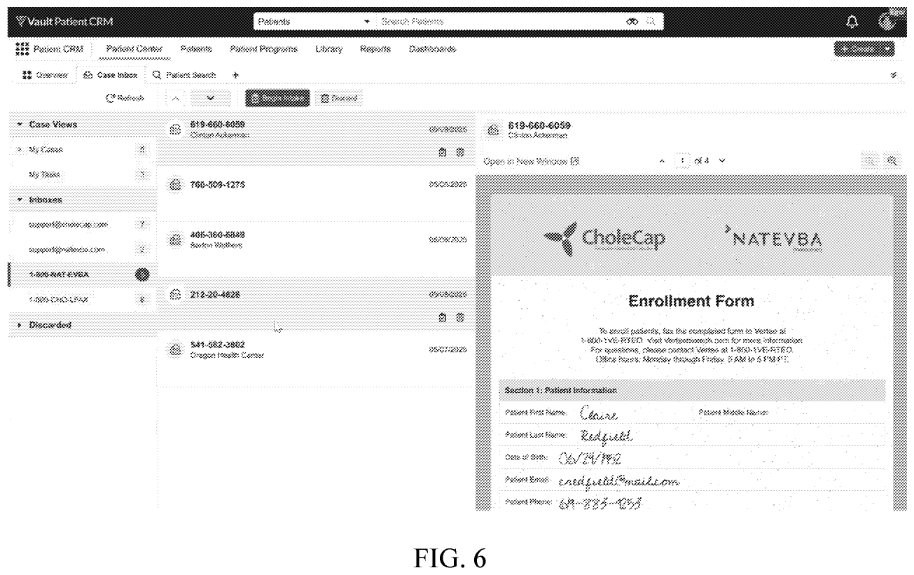

FIG. 6 depicts an exemplary user interface displaying a fax inbox with a fax document, according to aspects of the present disclosure. The interface includes a patient search function enabling users to search for existing patients by name or phone number (e.g., Claire Redfield 6198839253), with search results displayed. A “Create Case” option enables creation of new cases when no matching patient is found. The fax document from a sender (Clinton Ackerman) is displayed with an option to open in a new window.

In some aspects, the server may receive a fax document containing patient information. Fax transmission remains a communication channel through which healthcare providers and other parties submit patient enrollment forms, prescription information, and other patient-related documents to patient services programs. The server may receive inbound fax documents through a fax processing subsystem that converts received fax transmissions into digital document images for storage and processing.

In some implementations, a case inbox provides access to received fax documents and other case-related items. The case inbox may include multiple inbox views that enable users to organize and access cases based on different criteria. A my cases view may display cases assigned to the current user, enabling the user to view cases for which the user has ownership or responsibility. The case inbox may also include channel-specific inboxes that organize cases based on the communication channel through which the case originated or through which communications have been received.

In some cases, a fax inbox as a channel-specific inbox within the case inbox. The fax inbox may display fax documents that have been received and have not yet been processed. Users may navigate to the fax inbox to review received fax documents and determine whether to process the fax documents for patient intake. When a user selects a fax document from the fax inbox, the user may add the fax document to the user's queue for processing.

In some aspects, received fax images are viewed side-by-side alongside case details and intake forms. When a user accesses a fax document for processing, the user interface may display the fax document image in one region of the display and case details or intake form fields in another region of the display. The side-by-side arrangement may enable users to view the original fax document while entering or verifying data in the intake form without switching between different views or windows.

In some implementations, the user interface may display case details adjacent to the fax document image. Case details may include a case identifier, a case owner, a case severity level, a case type, and a case queue assignment. The case details may be displayed in a panel alongside the fax document image, enabling users to view case metadata while reviewing the fax document content.

In some cases, the user interface may display a patient intake form alongside the fax document image. The patient intake form may include fields for entering patient demographic information, contact information, and other patient data. Users may reference the fax document image displayed in the adjacent region while populating fields in the patient intake form. The side-by-side arrangement may reduce the need for users to memorize information from the fax document or to switch between different screens during the intake process.

In some aspects, the fax inbox may display a list of received fax documents with identifying information for each fax document. Identifying information may include a sender name, a sender fax number, a receipt timestamp, and a subject or description. Users may select a fax document from the list to view the fax document image and associated case details.

In some implementations, multiple channel-specific inboxes are supported in addition to the fax inbox. Channel-specific inboxes may include inboxes for email communications, phone communications, and other communication channels supported by the system. Each channel-specific inbox may display cases or communications received through the corresponding channel, enabling users to organize work based on communication channel.

In some cases, the case inbox may include filtering and sorting capabilities that enable users to locate cases based on various criteria. Users may filter cases by status, severity, type, queue assignment, or other case attributes. Users may also sort cases by receipt date, priority, or other ordering criteria. The filtering and sorting capabilities may enable users to prioritize work and locate cases that require attention.

In some aspects, when a user selects a fax document for processing, the server may create a case record associated with the fax document. The case record may store metadata about the fax document and may track the processing status of the fax document through the intake workflow. The case record may be stored in the first database along with patient data that is subsequently entered based on information extracted from the fax document.

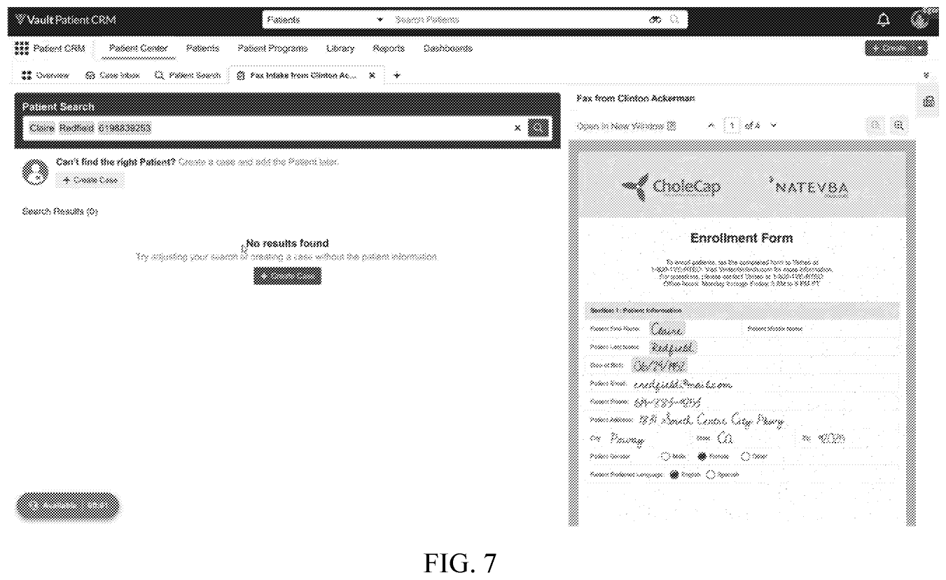

FIG. 7 depicts an exemplary user interface displaying a patient search and fax document with patient information, according to an embodiment. The interface displays a side-by-side view of a fax document (enrollment form) and case details. The case details panel displays case owner (Mike Torello), severity (Normal), type (Intake), queue (Default), and patient intake form fields including first name, last name, sex, home phone, email, date of birth, preferred communication, address, city, and state. The fax document displays an enrollment form with patient information sections.

In some aspects, CRM server 106 may receive a selection of a field in the patient intake form. As described above, the server may automatically populate a patient intake form with data fields extracted from a fax document using optical character recognition. Users may interact with the auto-populated patient intake form to verify that the extracted data fields have been correctly identified and populated. When a user selects a field in the patient intake form, the server may receive an indication of the selected field from the user computing device.

In some implementations, CRM server 106 may display a visual indication on the fax document corresponding to a location from which data populating the selected field was extracted. The visual indication may enable users to verify that the auto-populated value in the selected field corresponds to the correct source location in the original fax document. The visual indication may include a highlight, a border, a bounding box, or another graphical element that draws attention to the region of the fax document from which the data was extracted.

In some cases, the server may maintain a mapping between fields in the patient intake form and corresponding source locations in the fax document. When the server performs optical character recognition on the fax document and identifies data fields within the extracted text, the server may record the spatial coordinates or region identifiers associated with each extracted data field. The mapping may associate each field in the patient intake form with the corresponding source location in the fax document from which the data populating the field was extracted.

In some aspects, when CRM server 106 receives a selection of a field in the patient intake form, the server may retrieve the source location associated with the selected field from the mapping. CRM server 106 may then generate a visual indication at the retrieved source location on the fax document. The visual indication may be rendered on the fax document image displayed in the user interface, enabling the user to view the highlighted source area alongside the selected field in the patient intake form.

In some implementations, the side-by-side display arrangement described above may facilitate the verification process. The user interface may display the fax document image in one region of the display and the patient intake form in another region of the display. When a user selects a field in the patient intake form, the visual indication may appear on the fax document image in the adjacent region, enabling the user to compare the auto-populated value with the source text without switching between different views or windows.

In some cases, users may tab through fields in the patient intake form to sequentially verify auto-populated values. As the user navigates from one field to another field in the patient intake form, the server may update the visual indication on the fax document to highlight the source location corresponding to the currently selected field. The sequential verification process may enable users to confirm that each auto-populated value in the patient intake form matches the corresponding information in the original fax document.

In some aspects, the visual indication may include a highlight overlay applied to the source region on the fax document. The highlight overlay may use a color, transparency level, or visual effect that distinguishes the highlighted region from surrounding areas of the fax document. The highlight overlay may be sized and positioned to encompass the text or data element from which the auto-populated value was extracted.

In some implementations, the visual indication may include a bounding box drawn around the source region on the fax document. The bounding box may define the boundaries of the region from which the data populating the selected field was extracted. The bounding box may be rendered with a line style, color, or thickness that makes the bounding box visible against the fax document content.

In some cases, CRM server 106 may display visual indications for multiple fields simultaneously. When a user selects multiple fields in the patient intake form or when the user interface displays all auto-populated fields, the server may display visual indications on the fax document corresponding to the source locations for each of the selected or displayed fields. Each visual indication may be associated with a corresponding field in the patient intake form through color coding, numbering, or connecting lines.

In some aspects, the field selection and source highlighting functionality may reduce transcription errors during the patient intake process. By enabling users to verify that auto-populated values correspond to the correct source locations in the original fax document, the functionality may help users identify cases where the optical character recognition process has incorrectly extracted or associated data fields. Users may edit auto-populated values in the patient intake form when the visual indication reveals a discrepancy between the auto-populated value and the source text in the fax document.

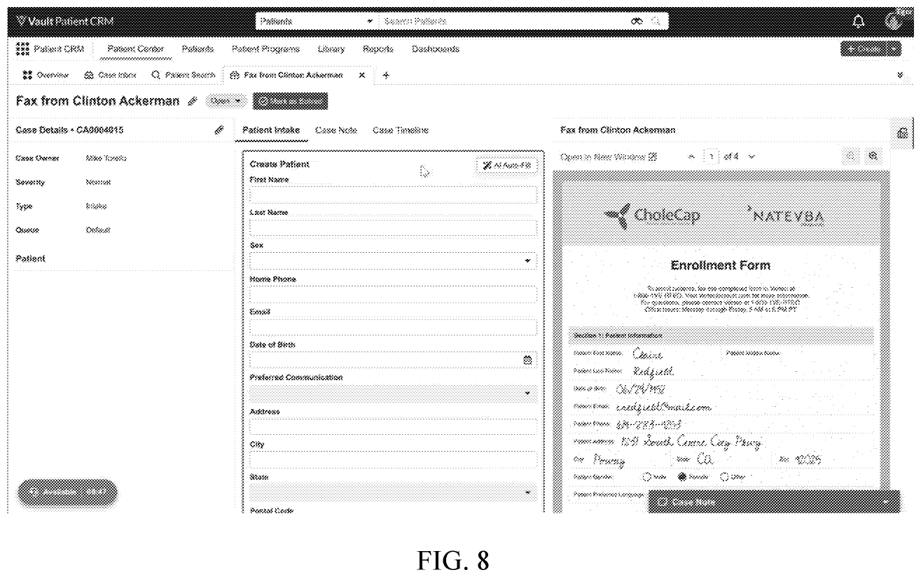

FIG. 8 depicts an exemplary user interface displaying a patient intake form with data fields, according to aspects of the present disclosure. The patient intake form interface displays empty fields for entering patient demographic information. The fax document enrollment form is displayed adjacent to the intake form, showing sections for patient information including patient first name (Claire), last name (Redfield), date of birth (Jun. 24, 1952), email (credfield@mail.com), phone (619-883-9253), address (1831 South Centre City Pkwy), city (Poway), state (CA), zip (92025), and gender selection options (Male, Female, Other).

In some aspects, CRM server 106 may perform optical character recognition on a fax document to extract text from the fax document. As described above, fax transmission remains a communication channel through which healthcare providers and other parties submit patient enrollment forms, prescription information, and other patient-related documents to patient services programs. The server may process received fax documents to extract text content for use in populating patient intake forms and other data entry workflows.

In some implementations, CRM server 106 may employ a multimodal large language model (MLLM) with vision capabilities for performing optical character recognition on fax documents. A multimodal large language model may combine natural language processing capabilities with image analysis capabilities, enabling the model to process document images and extract text content from the document images. The multimodal large language model may analyze visual features of a fax document image, identify text regions within the fax document image, and generate text output corresponding to the identified text regions.

In some cases, CRM server 106 may employ the multimodal large language model for optical character recognition to handle handwritten text recognition. Fax documents received from healthcare providers may contain handwritten information, such as handwritten patient names, handwritten signatures, handwritten dates, or handwritten notes. Handwritten text may exhibit variations in letter formation, spacing, and alignment that differ from printed or typed text. The multimodal large language model may process handwritten text within fax documents and extract text from the handwritten regions with accuracy that may exceed the accuracy achievable using rule-based optical character recognition engines.

In some aspects, CRM server 106 may use LLAVA (Large Language and Vision Assistant) as the multimodal large language model for document processing and text extraction. LLAVA may combine a vision encoder with a large language model to process image inputs and generate text outputs. The server may transmit fax document images to LLAVA for processing, and LLAVA may return extracted text corresponding to the content of the fax document images.

In some implementations, CRM server 106 may execute LLAVA on Ollama infrastructure for document processing operations. Ollama may provide a runtime environment for executing large language models, including multimodal large language models with vision capabilities. The server may communicate with Ollama infrastructure to submit fax document images for processing and receive extracted text results. The Ollama infrastructure may manage model loading, inference execution, and resource allocation for optical character recognition operations.

In some cases, the optical character recognition architecture may be designed to be flexible and modular. The modular architecture may allow the underlying large language model to be switched to different models without requiring changes to other components of the system. CRM server 106 may include an abstraction layer that interfaces with the multimodal large language model, where the abstraction layer defines a standard interface for submitting document images and receiving extracted text. Different multimodal large language models may be substituted behind the abstraction layer, enabling the system to adopt different models as model capabilities evolve or as different models become available.

In some aspects, the modular optical character recognition architecture may support configuration of the multimodal large language model used for text extraction. System administrators may configure the system to use a particular multimodal large language model for optical character recognition operations. The configuration may specify model parameters, inference settings, and connection details for the model infrastructure. The modular architecture may enable switching from one multimodal large language model to another multimodal large language model by updating the configuration without modifying application code.

In some implementations, CRM server 106 may perform optical character recognition on the fax document to extract text from the fax document by transmitting the fax document image to the multimodal large language model and receiving extracted text in response. The extracted text may include patient demographic information, contact information, prescription details, insurance information, and other data fields contained within the fax document. The server may use the extracted text to identify data fields for populating patient intake forms, as described above.

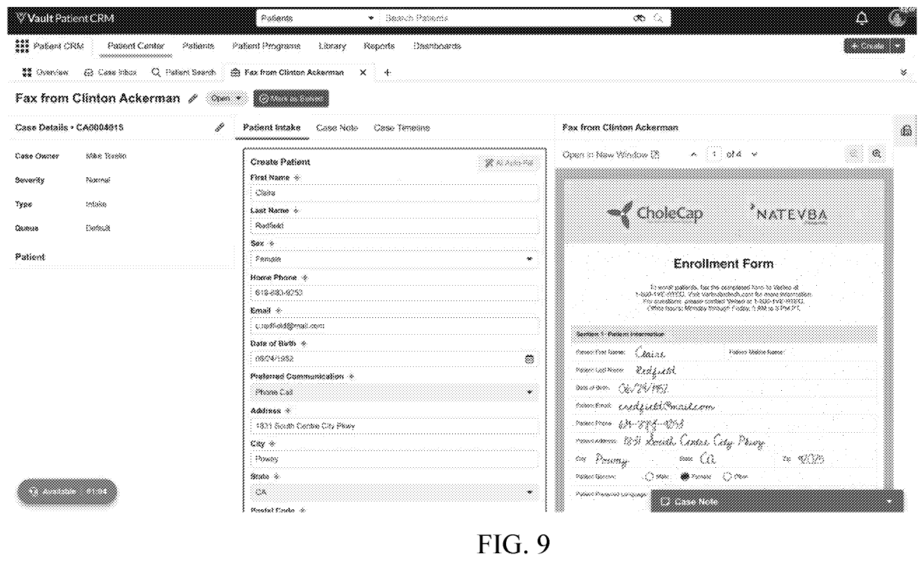

FIG. 9 depicts an exemplary user interface displaying a patient intake form with auto-populated data, according to an embodiment. The patient intake form displays fields automatically populated from the fax document using OCR extraction. Fields shown include first name (Claire), last name (Redfield), sex (Female), home phone (619-883-9253), email (c.redfield@mail.com), date of birth (Jun. 24, 1952), preferred communication (Phone Call), address (1831 South Centre City Pkwy), city (Poway), and state (CA). The fax document is displayed alongside showing the source enrollment form with corresponding fields from which data was extracted.

In some aspects, CRM server 106 may identify a plurality of data fields within extracted text obtained from a fax document. As described above, CRM server 106 may perform optical character recognition on a received fax document to extract text from the fax document. CRM server 106 may analyze the extracted text to identify data fields corresponding to patient information contained within the fax document. Data fields may include patient demographic information such as patient first name, patient last name, date of birth, gender, phone number, email address, physical address, city, state, and postal code. Data fields may also include medical information such as prescription details, treatment information, and insurance details.

In some implementations, CRM server 106 may identify data fields within the extracted text by recognizing field labels and associated values within the fax document. CRM server 106 may detect text patterns that correspond to form fields, such as labels followed by handwritten or typed values. CRM server 106 may use pattern matching, natural language processing, or machine learning techniques to associate extracted text values with corresponding data field types. For example, CRM server 106 may identify a sequence of digits in a format consistent with a phone number and associate the sequence with a phone number data field.

In some cases, CRM server 106 may identify data fields based on the spatial arrangement of text within the fax document. Enrollment forms and other patient intake documents may have predefined layouts in which patient information appears in designated regions of the document. CRM server 106 may use the spatial positions of extracted text elements to determine which data fields the text elements represent. CRM server 106 may maintain mappings between document regions and data field types to facilitate data field identification.

In some aspects, CRM server 106 may automatically populate a patient intake form with the plurality of data fields identified within the extracted text. The patient intake form may include input fields for entering patient demographic information, contact information, and other patient data. CRM server 106 may map each identified data field to a corresponding input field in the patient intake form and populate the input field with the extracted value. The automatic population of the patient intake form may reduce manual data entry and may reduce transcription errors that may occur during manual intake processes.

In some implementations, CRM server 106 may automatically populate a patient intake form with data extracted from the fax document. The patient intake form may display populated fields including first name, last name, sex, home phone, email, date of birth, preferred communication method, address, city, and state. Users may review the auto-populated values in the patient intake form and verify that the values match the information contained in the original fax document. Users may edit auto-populated values if corrections are needed before completing the intake process.

In some cases, CRM server 106 may display the fax document alongside the patient intake form in a side-by-side arrangement. The side-by-side display may enable users to compare the auto-populated values in the patient intake form with the corresponding information in the original fax document. Users may visually verify that the extracted data fields have been correctly identified and populated without switching between different views or windows.

In some aspects, CRM server 106 may include patient search functionality with three-point search criteria to identify whether a patient exists in a patient universe before creating new records. The patient universe may comprise patient records stored in the first database. When a user initiates patient intake based on a received fax document, the server may perform a patient search using search criteria derived from the fax document or entered by the user. The three-point search criteria may include a combination of patient identifying attributes such as patient name, phone number, and date of birth.

In some implementations, CRM server 106 may receive search criteria from a user computing device and query the first database to locate patient records matching the search criteria. The search criteria may include a patient name and a phone number, such as a search query containing both a patient name and an associated phone number. CRM server 106 may compare the search criteria against patient records stored in the first database and return matching patient records to the user computing device for display.

In some cases, CRM server 106 may display search results indicating whether matching patient records were found in the patient universe. If one or more matching patient records are found, the user may select an existing patient record to associate with the current intake case. If no matching patient records are found, the user may create a new patient record. The user interface may display a message indicating that no matching patient was found and may provide an option to create a new case with a new patient record.

In some aspects, the three-point search may reduce the creation of duplicate patient records in the first database. By searching for existing patient records before creating new records, the system may identify cases where a patient already exists in the patient universe. The user may then associate the intake case with the existing patient record rather than creating a duplicate record. The three-point search criteria may be configurable based on the requirements of the patient services program.

In some implementations, CRM server 106 may use data extracted from the fax document to pre-populate the patient search criteria. The server may extract patient name and phone number from the fax document using optical character recognition and use the extracted values as search criteria to query the first database. The server may display the search results to the user, enabling the user to determine whether the patient already exists in the patient universe before proceeding with patient record creation.

In some cases, the patient intake form may include fields for entering prescription information and insurance information in addition to patient demographic information. CRM server 106 may identify data fields within the extracted text corresponding to prescription details, such as medication name, dosage, and number of refills. The server may also identify data fields corresponding to insurance information, such as insurance provider name and policy details. CRM server 106 may automatically populate the patient intake form with the identified prescription and insurance data fields extracted from the fax document.

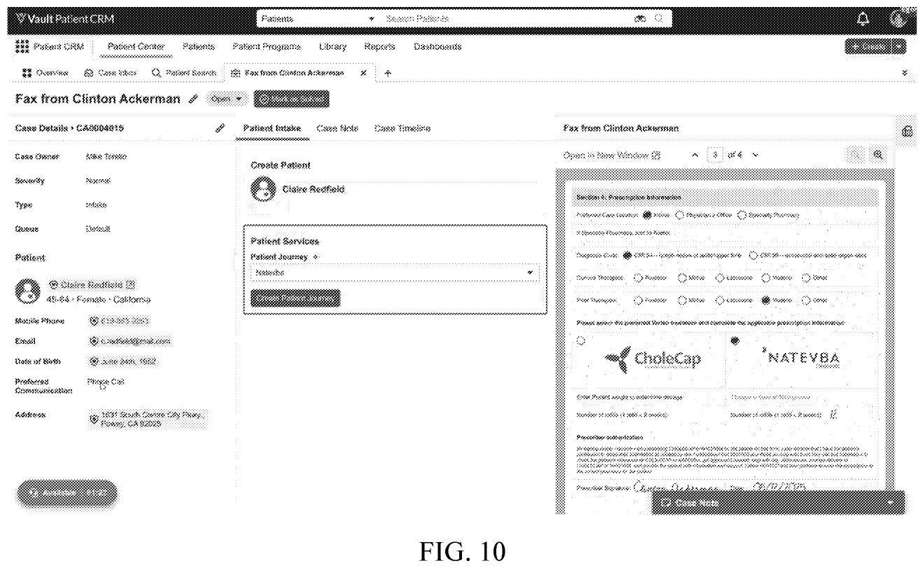

FIG. 10 depicts an exemplary user interface displaying patient services and patient journey information, according to aspects of the present disclosure. The interface displays patient journey selection, current therapies checkboxes (Restolar, Mithal, Labrinone, Vodoral, Other), prior therapies selection, treatment selection options (CholeCap, Natevba), dosage information (500 mg/dose), number of refills (12, where 1 refill=2 weeks), and a prescriber authorization section with signature confirmation text indicating the prescriber is prescribing the medication and has patient permission to share information as needed.

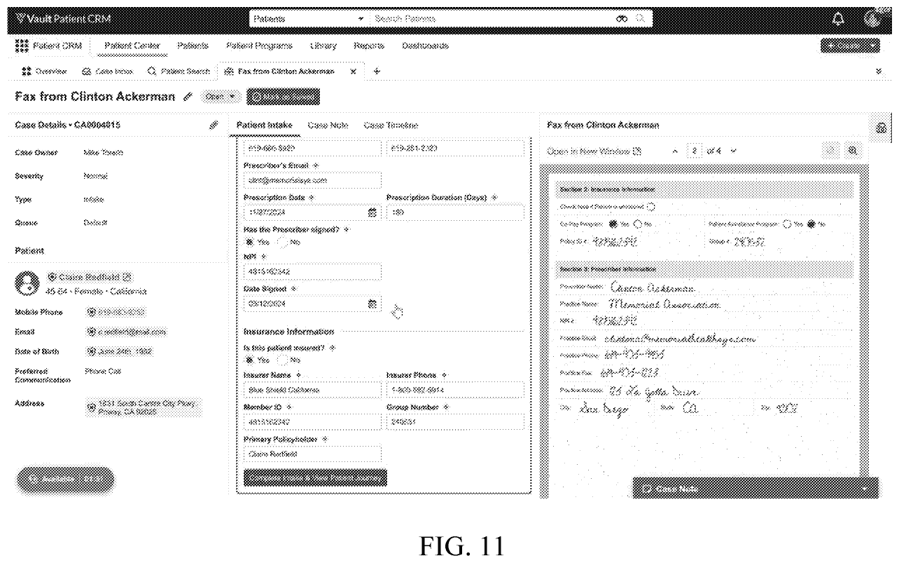

FIG. 11 depicts an exemplary user interface displaying patient intake with prescription and insurance information, according to an embodiment. The interface displays a case details panel with case owner (Mike Torello), severity (Normal), type (Intake), queue (Default), and patient information including mobile phone (619-883-9253), email (c.redfield@mail.com), date of birth (Jun. 24, 1952), preferred communication (Phone Call), and address (1831 South Centre City Pkwy., Poway, CA 92025). The interface includes prescription information fields and insurance details entry sections.

In some aspects, a user interface may include a visual indicator adjacent to fields containing at least one of protected health information or personally identifiable information. The visual indicator may identify fields that are subject to additional security handling relative to other fields displayed in the user interface. In some implementations, the visual indicator may comprise a shield icon displayed adjacent to or around fields containing protected health information or personally identifiable information. The shield icon may provide a visual cue to users that the associated field contains sensitive patient data.

In some cases, the visual indicator may be displayed as a border around the field containing protected health information or personally identifiable information. The border may distinguish fields containing sensitive patient data from other fields in the user interface that do not contain protected health information or personally identifiable information. In some implementations, the visual indicator may be rendered adjacent to the field label, adjacent to the field value, or surrounding the field input area.

In some aspects, the system may determine which fields to display with the visual indicator based on field-level configuration settings. A field on an object may be configured to indicate whether the field contains protected health information or personally identifiable information. When the field-level configuration setting indicates that a field contains protected health information or personally identifiable information, the user interface may display the visual indicator adjacent to the field. The field-level configuration may be established by system administrators during system setup or configuration.

In some implementations, displaying a visual indicator adjacent to fields in the user interface containing at least one of protected health information or personally identifiable information may enable users to identify sensitive data fields while viewing patient records. The visual indicator may appear on patient overview screens, patient intake forms, case detail views, and other user interface screens that display patient data retrieved from the first database.

In some cases, fields having the visual indicator may be associated with additional security handling. The additional security handling may include encryption of data associated with fields having the visual indicator during transmission between the server and user computing devices. The additional security handling may also include encryption at rest within the first database for data stored in fields designated as containing protected health information or personally identifiable information. In some aspects, the additional security handling may include masking of values in audit trails such that audit history records indicate that a change occurred to a field without revealing the actual values that were changed. For example, an audit trail entry may indicate that a field value was changed from one value to another value without displaying the actual protected health information or personally identifiable information values.

In some implementations, the visual indicator may be displayed consistently across different user interface views and screens. When a user navigates from a patient overview screen to a case detail screen or to a patient intake form, the visual indicator may appear adjacent to the same fields that contain protected health information or personally identifiable information. The consistent display of the visual indicator may enable users to recognize fields containing sensitive patient data regardless of the user interface context in which the fields are displayed.

In some aspects, fields designated as containing protected health information or personally identifiable information may be subject to additional security handling. As described above, a user interface may include a visual indicator adjacent to fields containing at least one of protected health information or personally identifiable information. Data associated with fields having the visual indicator may be encrypted during transmission between the server and user computing devices. The encryption during transmission may protect sensitive patient data as the data traverses network connections between the server and user computing devices.

In some implementations, data associated with fields having the visual indicator may also be encrypted at rest within the first database. The encryption at rest may protect sensitive patient data while the data is stored in the first database. The combination of encryption during transmission and encryption at rest may provide protection for protected health information and personally identifiable information throughout the data lifecycle.

In some cases, the system may mask protected health information and personally identifiable information values in audit trails. When a change is made to a field containing protected health information or personally identifiable information, the audit trail may record that a change occurred without revealing the actual values that were changed. For example, an audit history record may indicate that a field value was changed from a first masked value to a second masked value rather than displaying the actual protected health information or personally identifiable information values. The audit trail masking may enable tracking of changes to sensitive fields for compliance and accountability purposes while preventing exposure of actual patient data in audit records.

In some aspects, the system may mask protected health information and personally identifiable information values in administrative interfaces. A Vault Management Console (VMC) may provide administrative access to system data for super administrators. When a super administrator accesses patient data through the Vault Management Console, values in fields designated as containing protected health information or personally identifiable information may be masked. The masking in the Vault Management Console may prevent super administrators from viewing actual sensitive patient data even when the super administrators have administrative access to the system. The masked values may appear as asterisks or other placeholder characters in place of the actual protected health information or personally identifiable information values.

In some implementations, the designation of fields as containing protected health information or personally identifiable information may be configured at the field level on objects within the system. A field on an object may be configured with a setting indicating that the field contains protected health information or personally identifiable information. When the field-level setting indicates that a field contains protected health information or personally identifiable information, the system may apply the additional security handling to data stored in the field, including encryption during transmission, encryption at rest, audit trail masking, and administrative interface masking.

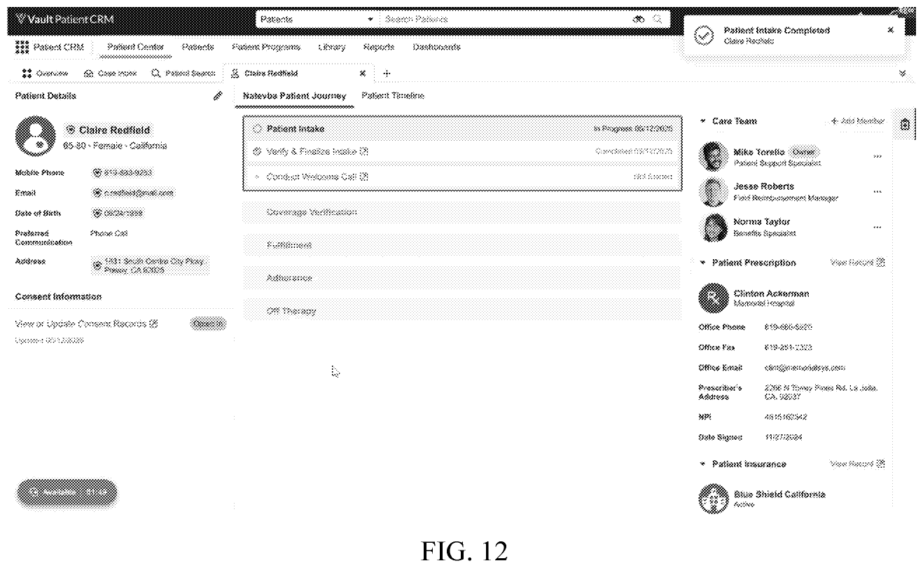

FIG. 12 depicts an exemplary user interface displaying a patient journey with workflow stages, according to aspects of the present disclosure. The interface displays workflow stages including Intake, Enrollment, Benefits Verification, Coverage Verification, Fulfillment, and Adherence. Each stage displays status indicators (Completed, In Progress, Not Started) with completion dates. The care team section displays team members with roles including Patient Support Specialist (Mike Torello), Benefits Specialist (Norma Taylor), and Field Reimbursement Manager. The prescriber information panel displays office phone (619-660-5920), fax (619-251-2323), email (clint@memorialsys.com), address (2266 N Torrey Pines Rd. La Jolla, CA, 92037), NPI (4815162342), and date signed (Nov. 27, 2024).

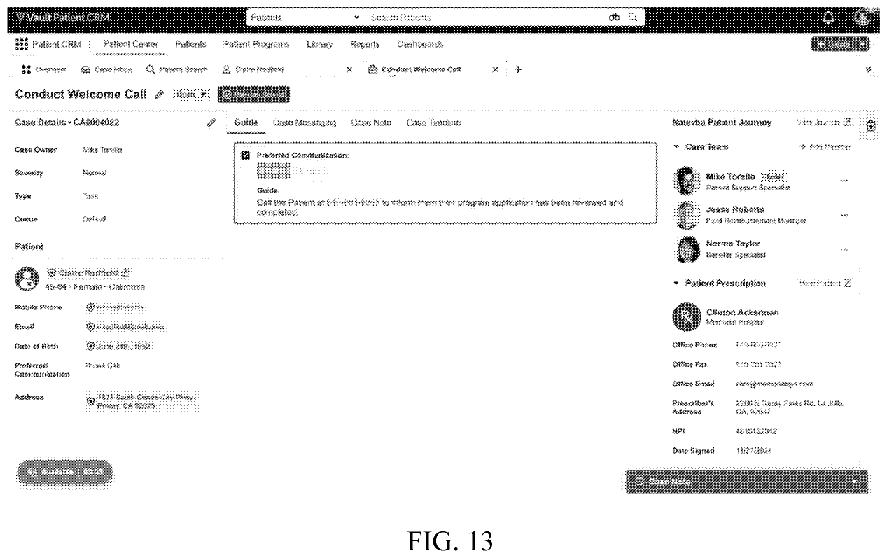

FIG. 13 depicts an exemplary user interface displaying a case task with communication guidance, according to an embodiment. The interface displays case details, a guide section, case messaging, case note entry, and case timeline. The interface includes a “Change Case State to Solved” option and a comment entry field with character count indicator (3162 characters left) and a “Mark Case as Solved” button. The care team panel displays team members including Patient Support Specialist (Mike Torello), Benefits Specialist (Norma Taylor), and prescriber information including Clinton Ackerman at Memorial Hospital with contact details.

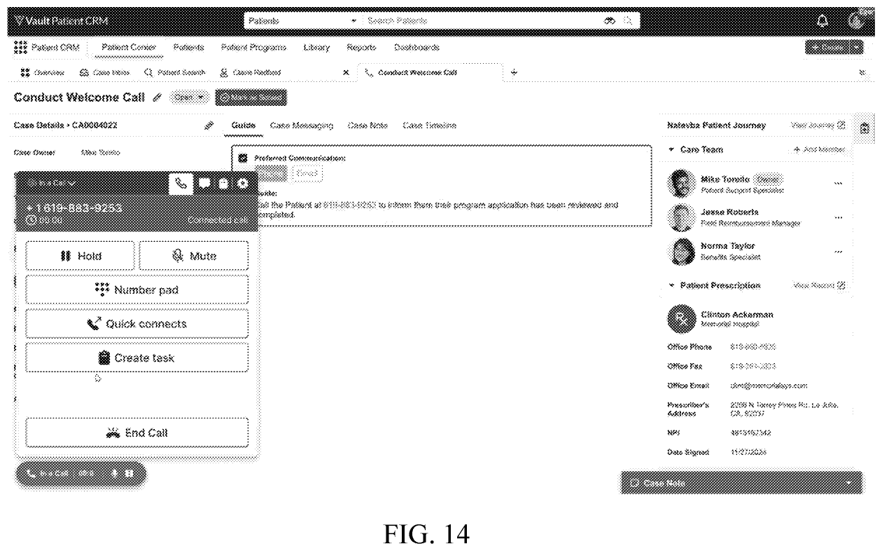

FIG. 14 depicts an exemplary user interface displaying an active call interface, according to aspects of the present disclosure. The interface displays a call status indicator showing availability status and call duration timer (04:03). The interface enables users to conduct phone communications with patients while viewing case information and patient details in adjacent panels.

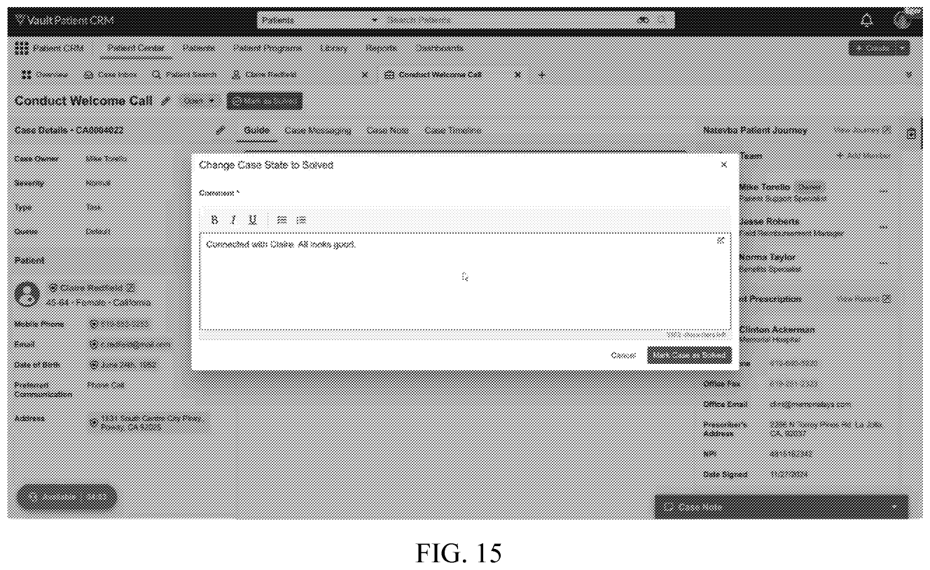

FIG. 15 depicts an exemplary user interface displaying a case comment entry interface, according to an embodiment. The interface displays a text field for entering case notes (e.g., “Connected with Claire. All looks good.”), a character count indicator, and a “Mark Case as Solved” button. The interface displays case details and care team information alongside the comment entry area, enabling users to document case interactions and update case status.

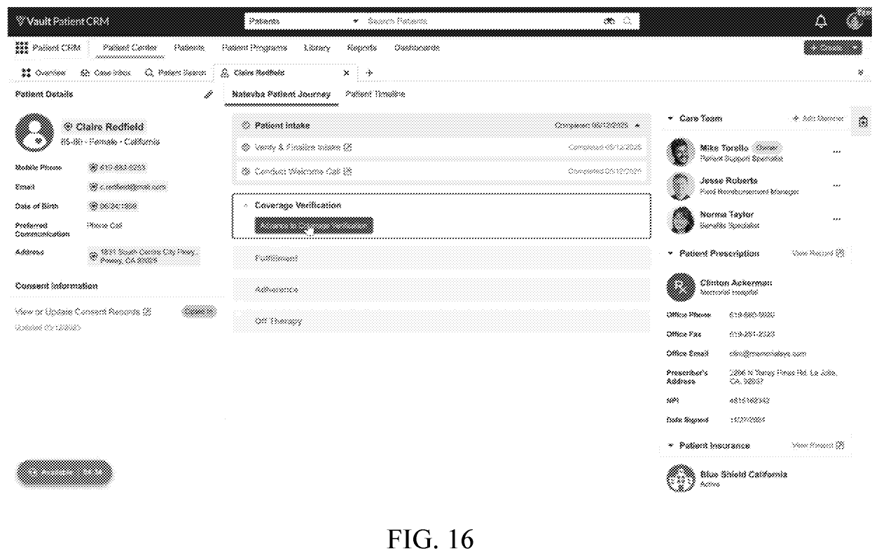

FIG. 16 depicts an exemplary user interface displaying a patient journey with completed workflow stages, according to aspects of the present disclosure. The interface displays completed workflow stages with completion dates. The patient consent information section includes a “View or Update Consent Records” option with an update timestamp (Updated May 12, 2025). Patient demographic information is displayed including mobile phone (619-883-9253), email (c.redfield@mail.com), date of birth (Jun. 24, 1959), preferred communication (Phone Call), and address. The workflow stage progression displays stages from Intake through Adherence with status indicators (Completed, In Progress, Not Started) for each stage.

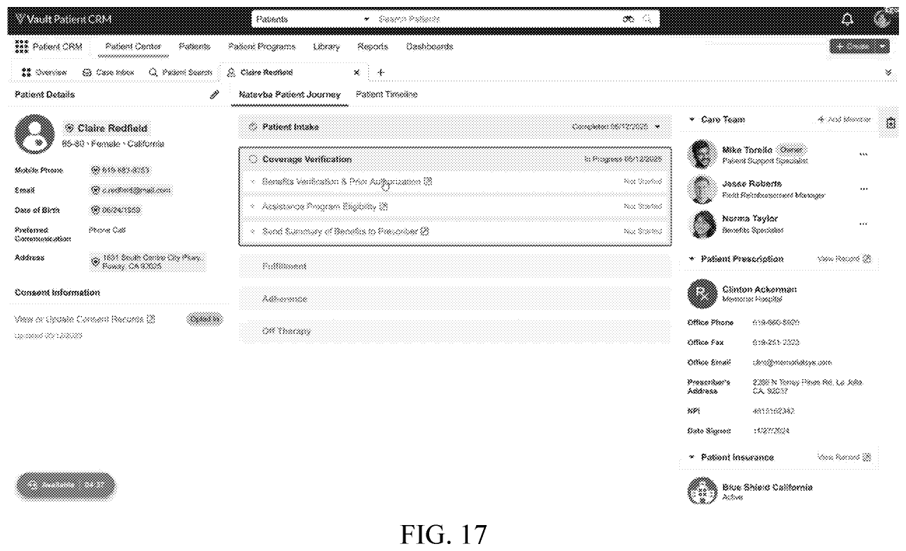

FIG. 17 depicts an exemplary user interface displaying a coverage verification workflow, according to an embodiment. The interface displays benefits verification task details, coverage outcome entry options, and insurance information. The interface enables users to record coverage determination outcomes and manage prior authorization workflows as part of the patient journey through the coverage verification stage.

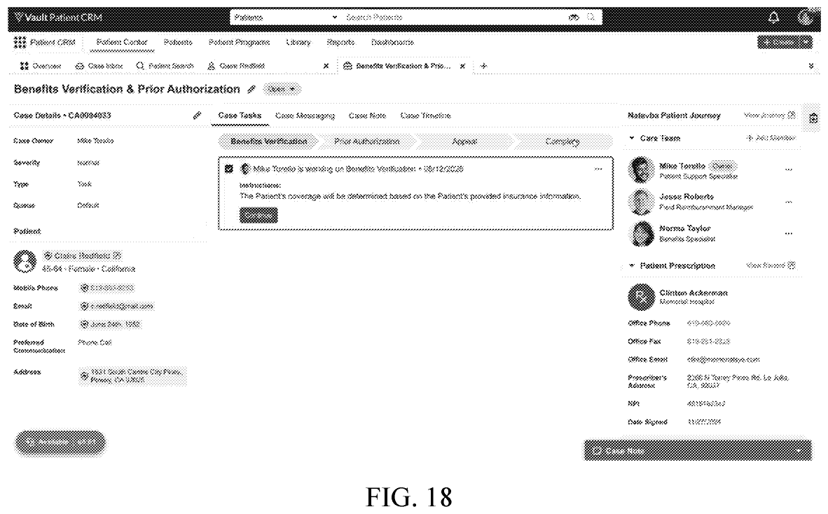

FIG. 18 depicts an exemplary user interface displaying a benefits verification task, according to aspects of the present disclosure. The interface displays task details, patient information, and benefits verification workflow steps. The interface includes options for recording verification outcomes and managing coverage-related tasks within the patient journey workflow.

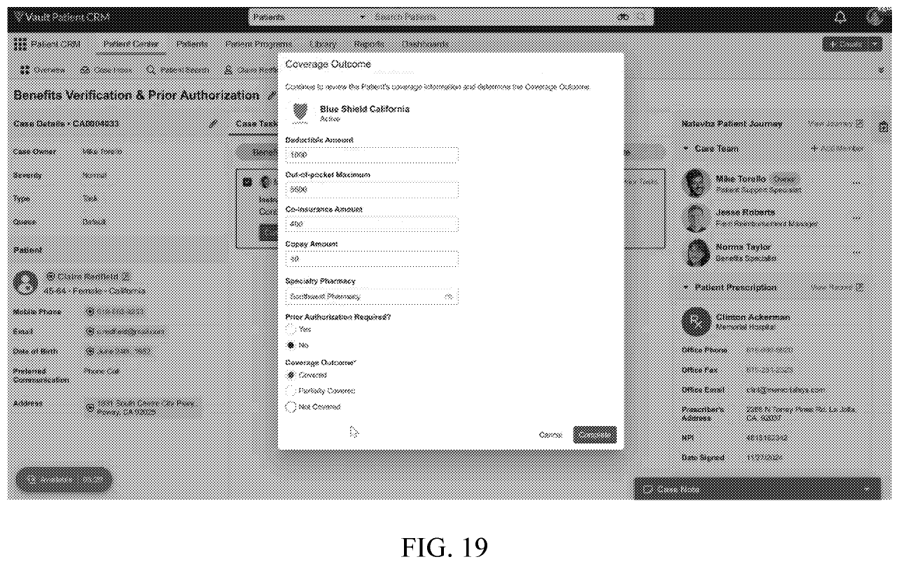

FIG. 19 depicts an exemplary user interface displaying a coverage outcome entry interface, according to an embodiment. The interface enables users to record coverage determination results. The interface displays coverage details including group number (240531), effective date (Jan. 1, 2025), and a coverage details table showing individual deductible ($1000 in-network, $2000 out-of-network), family deductible ($2000 in-network, $4000 out-of-network), and individual out-of-pocket maximum ($3000 in-network) with descriptions for each coverage element.

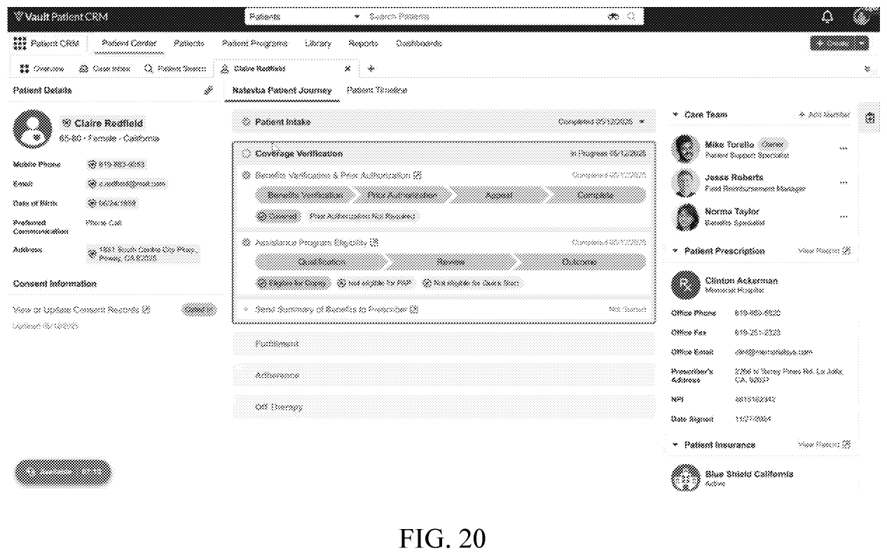

FIG. 20 depicts an exemplary user interface displaying a patient journey with coverage verification details, according to aspects of the present disclosure. The interface displays the completed coverage verification stage with detailed coverage information. The care team section displays team members including Patient Support Specialist (Mike Torello), Field Reimbursement Manager (Jesse Roberts), and Benefits Specialist (Norma Taylor). The prescriber information panel displays Clinton Ackerman at Memorial Hospital with contact details including office phone, fax, email, address, NPI (4815162342), and date signed (Nov. 27, 2024).

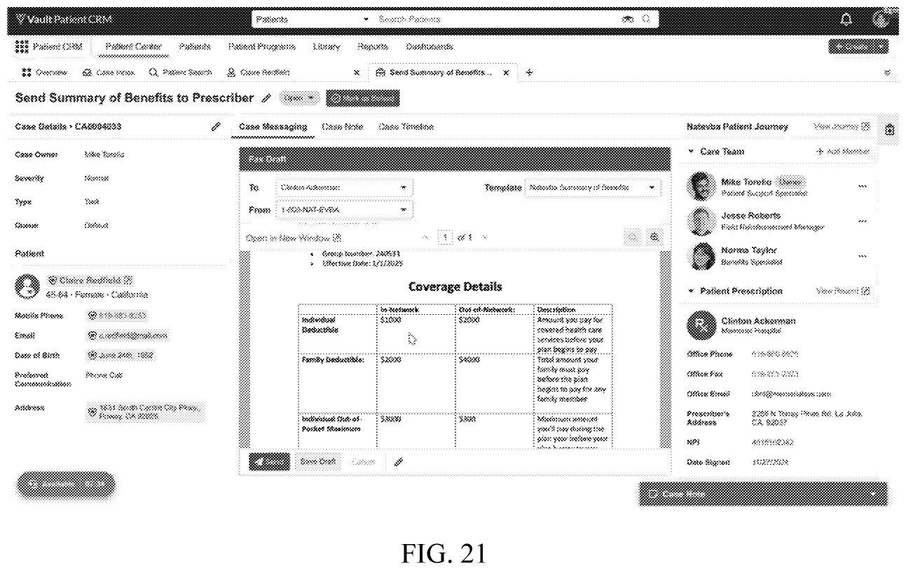

FIG. 21 depicts an exemplary user interface displaying a benefits summary fax draft, according to an embodiment. The interface displays a fax draft for sending benefits summary information to healthcare providers. The interface displays fax recipient (Clinton Ackerman), sender information (1-800-NAT-EVBA), template selection (Natevba Summary of Benefits), and a coverage details table showing individual deductible, family deductible, and out-of-pocket maximum amounts for in-network and out-of-network coverage with descriptions for each element.

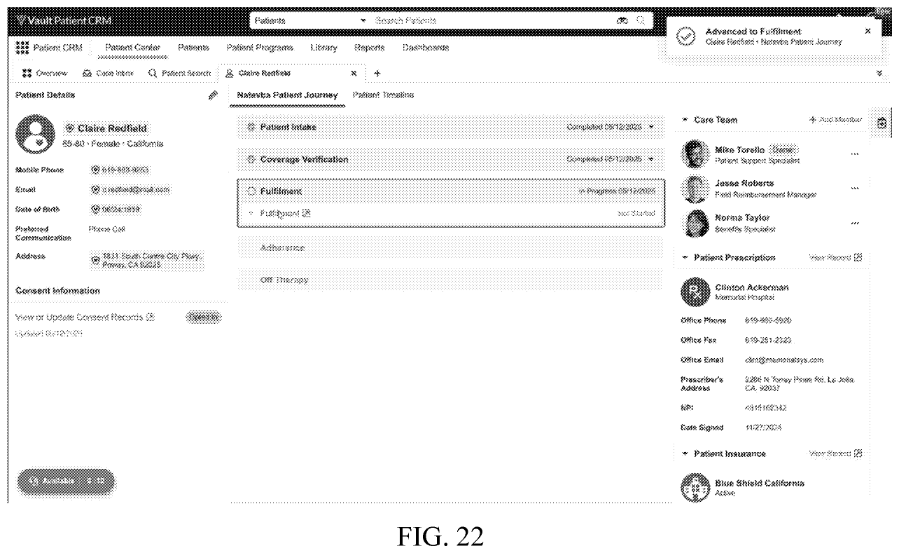

FIG. 22 depicts an exemplary user interface displaying a patient journey with fulfillment stage, according to aspects of the present disclosure. The interface displays the patient journey with progression through intake, enrollment, benefits verification, coverage verification, and fulfillment stages with status indicators and completion dates for each stage.

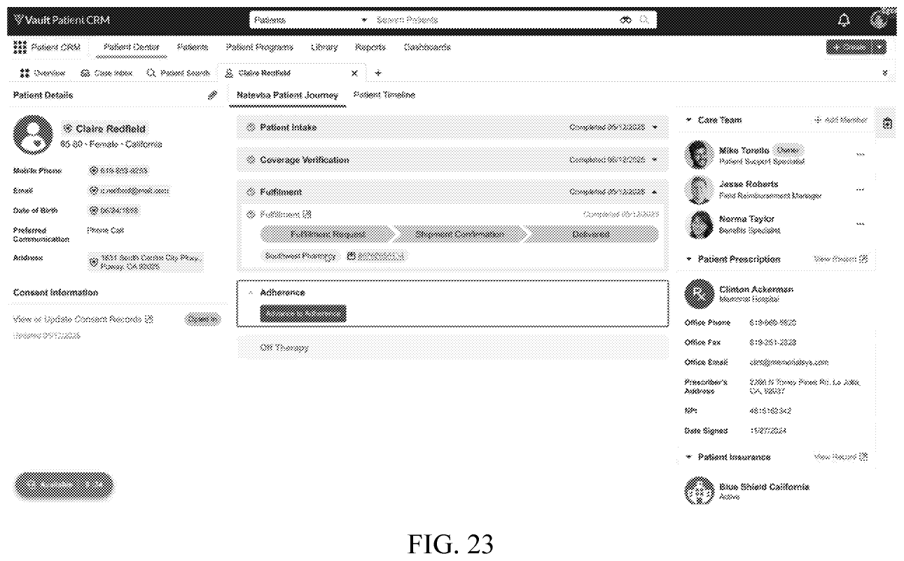

FIG. 23 depicts an exemplary user interface displaying a patient journey with fulfillment and shipment confirmation, according to an embodiment. The interface displays the fulfillment stage with shipment tracking information. Patient details are displayed including mobile phone (619-883-9253), email (c.redfield@mail.com), date of birth (Jun. 24, 1959), preferred communication, and address. The consent information section displays “View or Update Consent Records” with update timestamp. Care team members and prescriber information including prescription details are displayed.

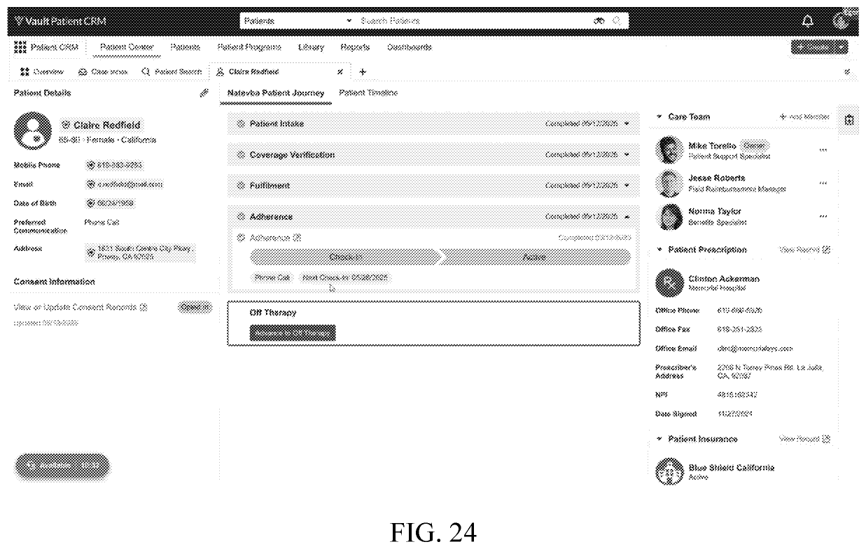

FIG. 24 depicts an exemplary user interface displaying a patient journey with adherence monitoring, according to aspects of the present disclosure. The interface displays the adherence monitoring stage showing completed workflow stages and adherence status indicators. Patient timeline information and care team details are displayed alongside patient demographic information including mobile phone, email, date of birth, preferred communication, and address. The prescriber information panel displays contact details, NPI, and prescription information.

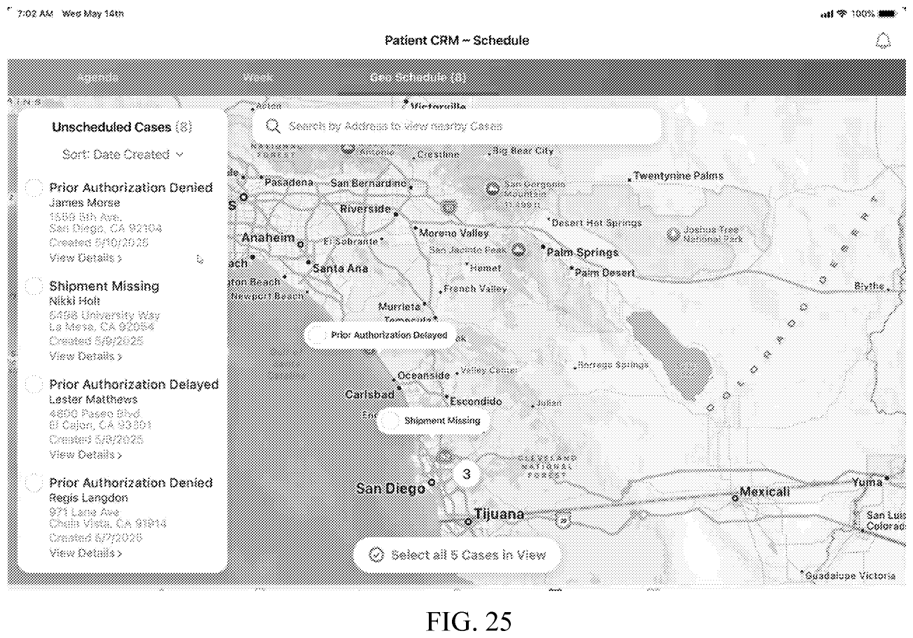

FIG. 25 depicts an exemplary user interface displaying a geographic scheduling map with patient cases, according to an embodiment. The geographic map interface displays patient case locations using pins or markers indicating locations associated with patient cases. The interface enables field reimbursement managers to visualize case distribution across their territory and select cases for scheduling.

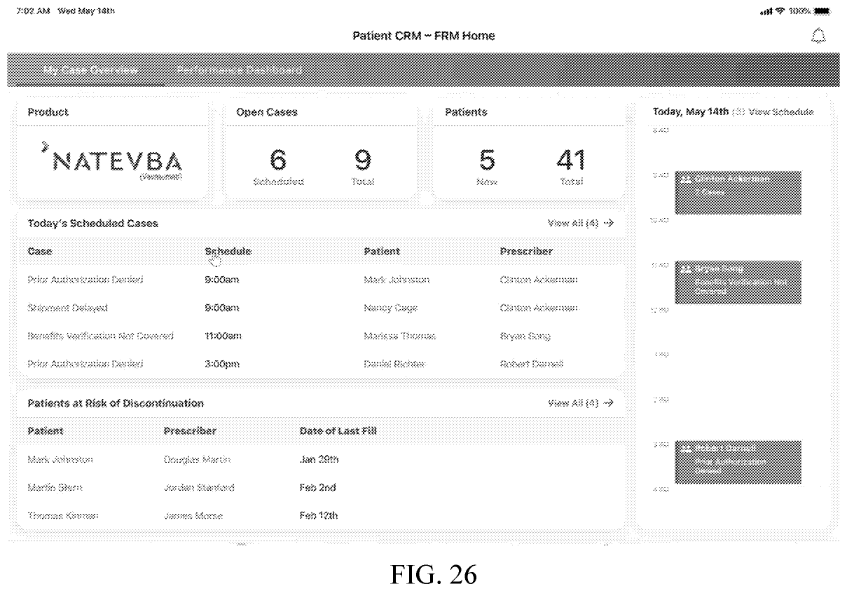

FIG. 26 depicts an exemplary user interface displaying a field representative home dashboard, according to aspects of the present disclosure. The FRM home dashboard displays a case overview with open cases count (6), scheduled cases, total cases (9), new patients count (5), and total patients (41). The dashboard displays product information (Natevba/Vevasumab), today's scheduled cases list showing case type (Prior Authorization Denied, Shipment Delayed, Benefits Verification Not Covered), patient name (Mark Johnston, Nancy Cage, Marissa Thomas, Daniel Richter), and prescriber (Clinton Ackerman, Bryan Song, Robert Darnell). A patients at risk of discontinuation list displays patient names and prescribers.

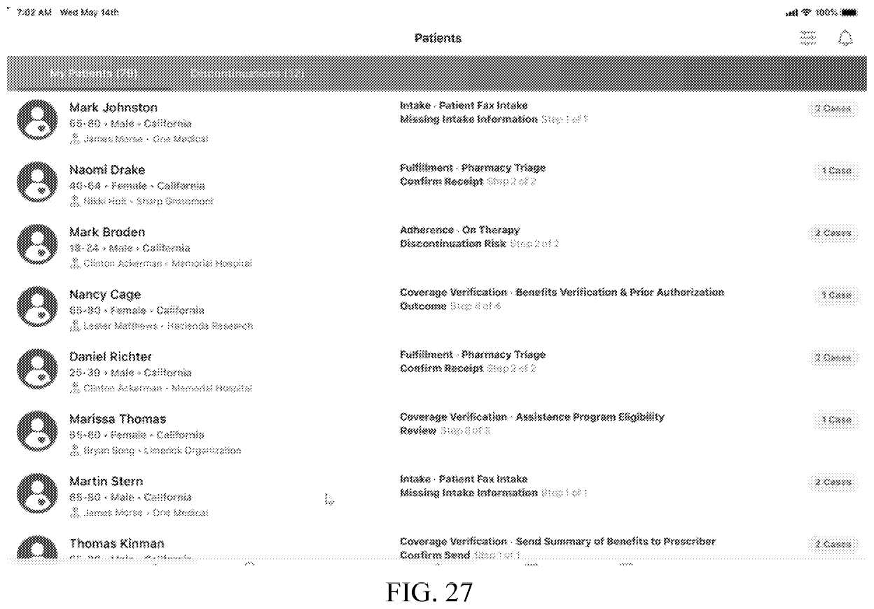

FIG. 27 depicts an exemplary user interface displaying a patient list with workflow status indicators, according to an embodiment. The patient list interface displays patients with workflow status indicators. Each patient entry shows patient name (Mark Johnston, Naomi Drake), age range (65-80, 40-64), gender (Male, Female), location (California), prescriber information (James Morse at One Medical, Nikki Holt at Sharp Grossmont), current workflow stage (Intake, Fulfillment, Adherence, Coverage Verification), specific task within the stage (Patient Fax Intake, Pharmacy Triage, Discontinuation Risk, Benefits Verification), and step progress indicator (e.g., Step 1 of 1, Step 2 of 2, Step 4 of 4).

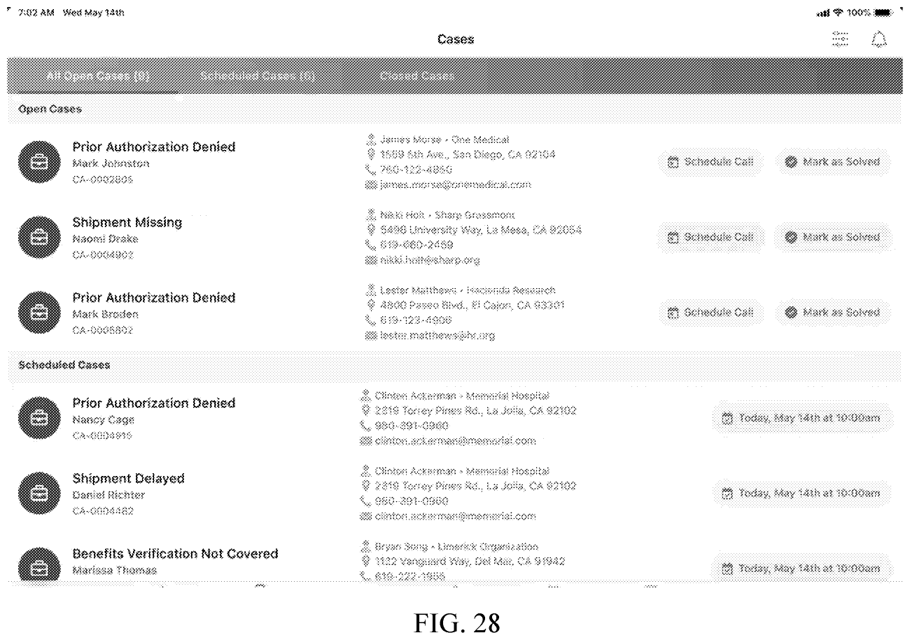

FIG. 28 depicts an exemplary user interface displaying open and scheduled cases, according to aspects of the present disclosure. The cases interface displays open cases and scheduled cases in separate sections. Open cases display case identifier (CA-0002805, CA-0004902, CA-0005802), case type (Shipment Missing, Prior Authorization Denied, Benefits Verification Not Covered), patient name (Mark Johnston, Naomi Drake, Mark Broden), and prescriber contact information including name, address, phone, and email. Scheduled cases display scheduled date and time (Today, May 14th at 10:00 am) with options to schedule call or mark as solved.

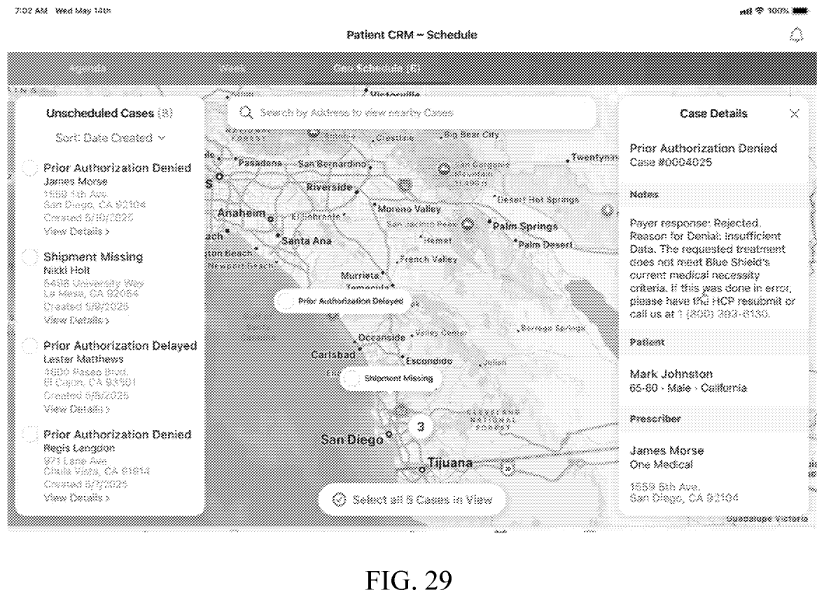

FIG. 29 depicts an exemplary user interface displaying a geographic map with case details, according to an embodiment. The geographic map interface displays case locations with a case detail overlay. The map shows pins indicating case locations and displays case information when a location is selected. The interface enables field representatives to view case distribution and access case details from the map view.

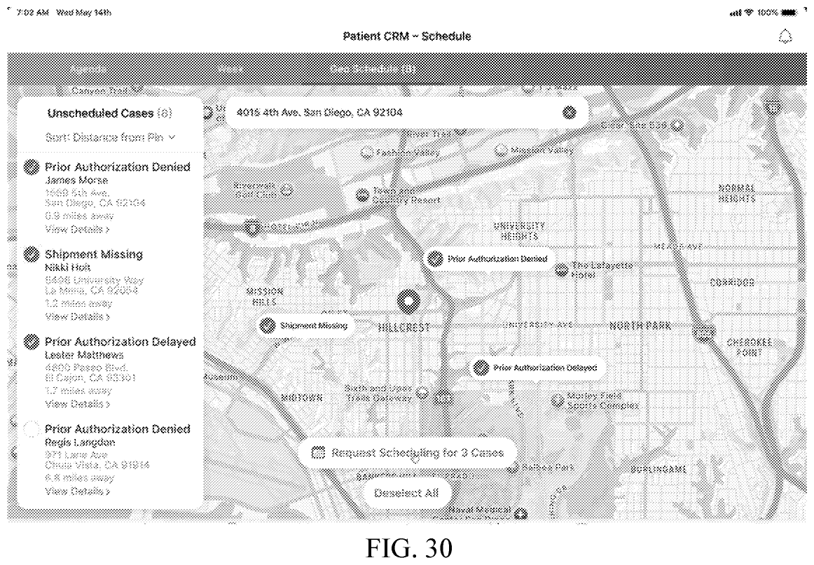

FIG. 30 depicts an exemplary user interface displaying a geographic map with case selection for scheduling, according to aspects of the present disclosure. The geographic map interface includes case selection functionality for grouped scheduling. The interface enables users to select multiple cases within a geographic area by dropping a pin and selecting surrounding cases. Selected cases are highlighted for batch scheduling operations, enabling field reimbursement managers to efficiently schedule visits to multiple nearby patient cases.

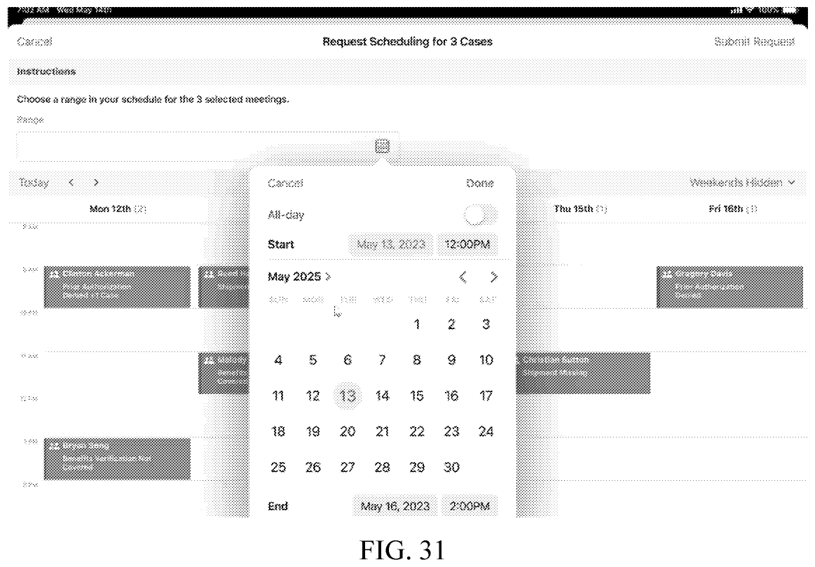

FIG. 31 depicts an exemplary user interface displaying a scheduling request interface, according to an embodiment. The scheduling request interface enables requesting scheduling for multiple cases (e.g., “Request Scheduling for 3 Cases”). The interface displays a calendar view with date range selection, start date picker (May 13, 2023), end date picker (May 16, 2023), and time selection (12:00 PM to 2:00 PM). The interface shows scheduled appointments on the calendar including prescriber names (Clinton Ackerman, Bryan Song, Gregory Davis, Christian Sutton) and case types (Prior Authorization Denied, Shipment Missing). Submit Request and Cancel buttons enable submission or cancellation of scheduling requests.

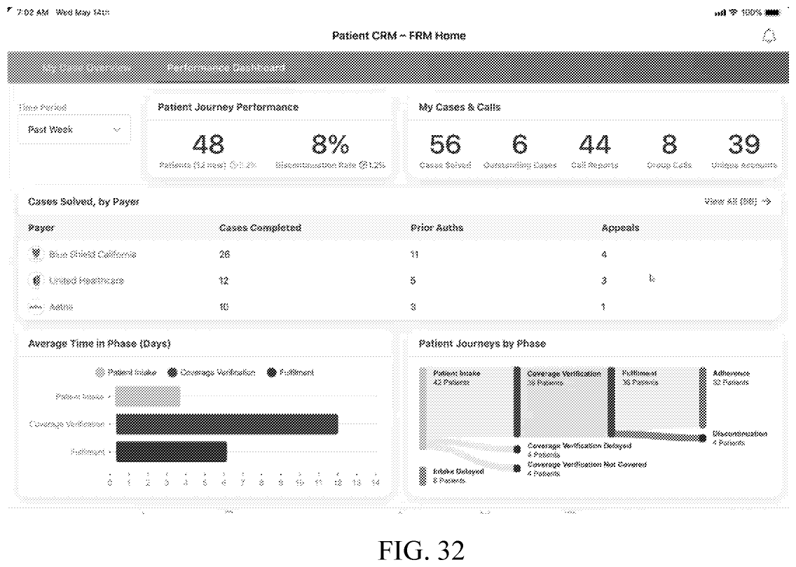

FIG. 32 depicts an exemplary user interface displaying a field representative performance dashboard, according to aspects of the present disclosure. The FRM performance dashboard displays performance metrics and analytics. The dashboard provides visibility into case resolution rates, patient outcomes, and territory performance indicators for field reimbursement managers.

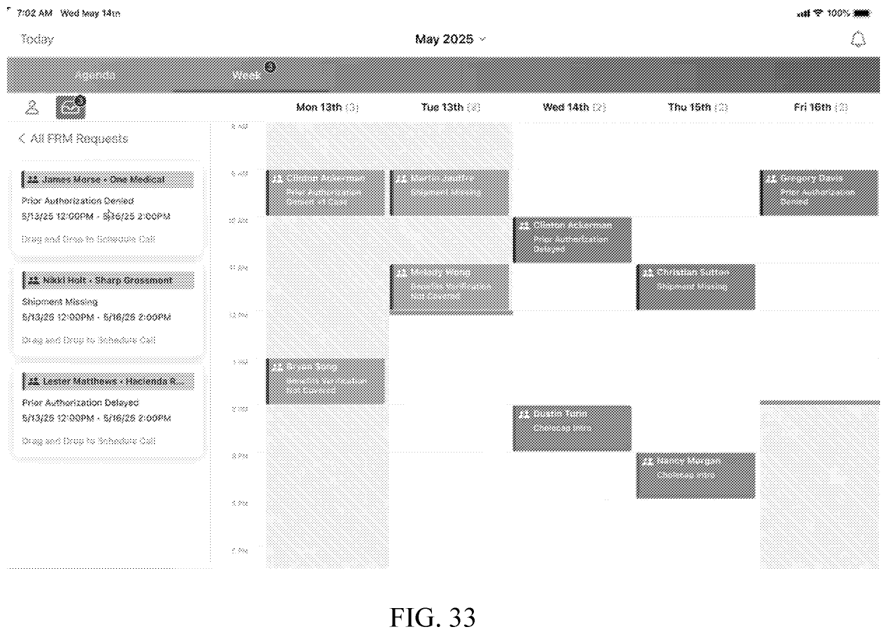

FIG. 33 depicts an exemplary user interface displaying a weekly schedule calendar view, according to an embodiment. The weekly schedule calendar view displays scheduled appointments across the week. The calendar displays time slots (8 AM through 2 PM), scheduled cases with prescriber names and case types, and enables navigation between weeks. A “Weekends Hidden” option is displayed. The interface displays case details including prescriber name (Clinton Ackerman, Bryan Song, Gregory Davis, Christian Sutton) and case type (Prior Authorization Denied, Shipment Missing, Benefits Verification Not Covered) for each scheduled appointment.

In some aspects, a server may identify a plurality of data fields within extracted text obtained from a fax document. As described above, the server may perform optical character recognition on a received fax document to extract text from the fax document. The server may analyze the extracted text to identify data fields corresponding to patient information contained within the fax document. Data fields may include patient demographic information such as patient first name, patient last name, date of birth, gender, phone number, email address, physical address, city, state, and postal code. Data fields may also include medical information such as prescription details, treatment information, and insurance details.

In some implementations, the server may identify data fields within the extracted text by recognizing field labels and associated values within the fax document. The server may detect text patterns that correspond to form fields, such as labels followed by handwritten or typed values. The server may use pattern matching, natural language processing, or machine learning techniques to associate extracted text values with corresponding data field types. For example, the server may identify a sequence of digits in a format consistent with a phone number and associate the sequence with a phone number data field.

In some cases, the server may identify data fields based on the spatial arrangement of text within the fax document. Enrollment forms and other patient intake documents may have predefined layouts in which patient information appears in designated regions of the document. The server may use the spatial positions of extracted text elements to determine which data fields the text elements represent. The server may maintain mappings between document regions and data field types to facilitate data field identification.

In some aspects, the server may automatically populate a patient intake form with the plurality of data fields identified within the extracted text. The patient intake form may include input fields for entering patient demographic information, contact information, and other patient data. The server may map each identified data field to a corresponding input field in the patient intake form and populate the input field with the extracted value. The automatic population of the patient intake form may reduce manual data entry and may reduce transcription errors that may occur during manual intake processes.

In some implementations, the server may automatically populate a patient intake form with data extracted from the fax document. The patient intake form may display populated fields including first name, last name, sex, home phone, email, date of birth, preferred communication method, address, city, and state. Users may review the auto-populated values in the patient intake form and verify that the values match the information contained in the original fax document. Users may edit auto-populated values if corrections are needed before completing the intake process.

In some cases, the server may display the fax document alongside the patient intake form in a side-by-side arrangement. The side-by-side display may enable users to compare the auto-populated values in the patient intake form with the corresponding information in the original fax document. Users may visually verify that the extracted data fields have been correctly identified and populated without switching between different views or windows.

In some aspects, the system may include patient search functionality with three-point search criteria to identify whether a patient exists in a patient universe before creating new records. The patient universe may comprise patient records stored in a first database. When a user initiates patient intake based on a received fax document, the server may perform a patient search using search criteria derived from the fax document or entered by the user. The three-point search criteria may include a combination of patient identifying attributes such as patient name, phone number, and date of birth.

In some implementations, the server may receive search criteria from a user computing device and query the first database to locate patient records matching the search criteria. The search criteria may include a patient name and a phone number, such as a search query containing both a patient name and an associated phone number. The server may compare the search criteria against patient records stored in the first database and return matching patient records to the user computing device for display.

In some cases, the server may display search results indicating whether matching patient records were found in the patient universe. If one or more matching patient records are found, the user may select an existing patient record to associate with the current intake case. If no matching patient records are found, the user may create a new patient record. The user interface may display a message indicating that no matching patient was found and may provide an option to create a new case with a new patient record.

In some aspects, the three-point search may reduce the creation of duplicate patient records in the first database. By searching for existing patient records before creating new records, the system may identify cases where a patient already exists in the patient universe. The user may then associate the intake case with the existing patient record rather than creating a duplicate record. The three-point search criteria may be configurable based on the requirements of the patient services program.

In some implementations, the server may use data extracted from the fax document to pre-populate the patient search criteria. The server may extract patient name and phone number from the fax document using optical character recognition and use the extracted values as search criteria to query the first database. The server may display the search results to the user, enabling the user to determine whether the patient already exists in the patient universe before proceeding with patient record creation.

In some cases, the patient intake form may include fields for entering prescription information and insurance information in addition to patient demographic information. The server may identify data fields within the extracted text corresponding to prescription details, such as medication name, dosage, and number of refills. The server may also identify data fields corresponding to insurance information, such as insurance provider name and policy details. The server may automatically populate the patient intake form with the identified prescription and insurance data fields extracted from the fax document.

In some aspects, the system may include an audit trail that tracks communications and associated data. The audit trail may track exact content of communications as the communications were sent by a user, such as a pharmaceutical representative or other personnel. The audit trail may also track metadata about the communications, where metadata may include timestamps, sender identifiers, recipient identifiers, communication channels, and other attributes associated with the communications. In some implementations, the audit trail may track information regarding content accessed by recipients, such as healthcare providers (HCPs), enabling the system to record which content items were viewed, downloaded, or otherwise accessed by recipients of communications.

In some cases, the audit trail may be stored in a controlled content repository. The controlled content repository may store approved content, customer profile information, customer preferences, and regulatory limitations and requirements. The audit trail stored in the controlled content repository may provide a record of communications that enables organizations to demonstrate compliance with applicable regulations and internal policies.

In some implementations, the system may include a regulatory compliance engine to facilitate regulatory compliance. The regulatory compliance engine may include audit trail systems that record changes to documents, communications, and other data within the system. The regulatory compliance engine may also include electronic signatures systems that enable users to apply electronic signatures to documents and communications in a manner that satisfies regulatory requirements. The regulatory compliance engine may further include system traceability functionality to comply with government regulations, such as 21 CFR Part 11, Annex 11, and GXP-related requirements.

In some aspects, the regulatory compliance engine may include processors for developing metadata surrounding document and project folder accesses. The metadata may record which users accessed which documents, when the accesses occurred, and what actions were performed during the accesses. From a regulatory compliance standpoint, the metadata may enable verification that accesses to documents and content have been permitted in accordance with applicable access controls and authorization rules.

In some cases, the regulatory compliance engine may include prevalidation functionality to build controlled content in support of installation qualification (IQ) and operational qualification (OQ). The prevalidation functionality may assist organizations in validating that the system operates in accordance with applicable regulatory requirements and organizational policies.

In some implementations, the system may include a reporting engine that reports on documents, document properties, and a complete audit trail of changes. The reporting engine may generate reports that show end users and management how content moves through a life cycle over time. The reports may enable tracking of planned activities versus actual activities and may enable identification of process bottlenecks. The reporting engine may include processors for developing and reporting life cycle and document management reporting based on stored project data and access metadata relative to documents, forms, and other communications stored in the controlled content repository.

In some aspects, the audit trail may record changes to fields containing protected health information or personally identifiable information in a masked format. When a change is made to a field designated as containing protected health information or personally identifiable information, the audit trail may record that a change occurred without revealing the actual values that were changed. The masking of protected health information and personally identifiable information in audit trails may enable tracking of changes for compliance and accountability purposes while preventing exposure of actual patient data in audit records.

In some aspects, a server may receive geographic location data associated with a plurality of patient cases. Patient cases stored in a first database may include location information associated with patients, healthcare providers, or both. Geographic location data may include address information, postal codes, geographic coordinates, or other location identifiers that enable the server to determine the geographic positions of patient cases. The server may receive geographic location data when patient cases are created during intake processes, when patient records are updated with address information, or when healthcare provider location data is retrieved from a second database.

In some implementations, the server may display a map interface depicting locations associated with the plurality of patient cases. The map interface may render a geographic map with visual markers indicating the locations of patient cases assigned to a user, such as a field reimbursement manager. The map interface may display patient case locations using pins, markers, or other graphical indicators positioned at geographic coordinates corresponding to patient or healthcare provider addresses associated with the patient cases.

In some cases, the map interface may enable users to visualize case distribution across a territory. Field reimbursement managers may cover large geographic territories, such as an entire state, and the map interface may provide a visual representation of how patient cases are distributed across the territory. The map interface may enable users to identify geographic patterns in case distribution, including areas with higher concentrations of patient cases and areas with fewer patient cases.

In some aspects, the map interface may enable visualization of case clusters across territories. A case cluster may comprise a group of patient cases located in geographic proximity to each other. The map interface may display case clusters by rendering multiple case markers in close proximity on the map, enabling users to identify areas where multiple patient cases are concentrated. Users may observe case clusters when viewing the map interface at different zoom levels.

In some implementations, the map interface may enable visualization of hot spots across territories. A hot spot may comprise a geographic area with a concentration of patient cases that may warrant attention or scheduling priority. The map interface may enable users to identify hot spots by displaying case markers in a manner that reveals areas with elevated case density. Users may use the hot spot visualization to prioritize travel and scheduling decisions based on geographic concentrations of patient cases.

In some cases, the map interface may support zoom functionality that enables users to view patient case locations at different levels of geographic detail. Users may zoom out to view case distribution across an entire territory, such as a state or region, and may zoom in to view case locations within a specific city or neighborhood. The zoom functionality may enable users to transition between high-level territory views and detailed local views of patient case locations.

In some aspects, the map interface may enable users to select individual patient cases by interacting with case markers displayed on the map. When a user selects a case marker on the map interface, the server may retrieve case details associated with the selected patient case and display the case details in a panel or overlay adjacent to the map. The case details may include patient information, case type, case status, and healthcare provider information associated with the selected patient case.

In some implementations, the map interface may display case markers with visual attributes that indicate case characteristics. Case markers may be rendered with different colors, shapes, or icons based on case type, case status, case severity, or other case attributes. The visual differentiation of case markers may enable users to identify cases with particular characteristics when viewing the map interface.

In some cases, the map interface may be accessible through a geographic scheduling tab or view within a client application. Field reimbursement managers and other users may navigate to the geographic scheduling tab to access the map interface and view patient case locations across their assigned territories. The geographic scheduling tab may provide access to map-based visualization and scheduling functionality in addition to list-based case views available elsewhere in the client application.

In some aspects, the server may receive a selection of a geographic area on the map interface. As described above, the map interface may display patient case locations using visual markers positioned at geographic coordinates corresponding to patient or healthcare provider addresses associated with patient cases. Users may select a geographic area on the map interface to identify patient cases located within the selected geographic area for scheduling purposes.

In some implementations, the map interface may support a touch-and-hold gesture for selecting a geographic area. When a user performs a touch-and-hold gesture on the map interface, such as by pressing and holding a finger on a touchscreen display of a user computing device, the server may receive an indication of the touch-and-hold gesture along with the geographic coordinates corresponding to the location of the gesture on the map interface. In response to the touch-and-hold gesture, the server may drop a pin at the indicated location on the map interface. The pin may serve as a reference point for defining a geographic area surrounding the pin location.

In some cases, the pin dropped in response to the touch-and-hold gesture may define a geographic area encompassing patient cases located in proximity to the pin location. The geographic area may be defined as a radius around the pin location, a bounding region around the pin location, or another geographic boundary that encompasses nearby patient case locations. The server may determine which patient cases are located within the geographic area defined by the pin.

In some aspects, the server may identify patient cases located within the selected geographic area for scheduling. When a pin is dropped on the map interface, the server may query patient case location data to identify patient cases having geographic coordinates that fall within the geographic area defined by the pin. The server may compare the geographic coordinates of patient case locations against the boundaries of the selected geographic area to determine which patient cases are located within the selected geographic area.

In some implementations, the map interface may enable selection of patient cases within a geographic area for grouped scheduling. After a pin is dropped and patient cases within the surrounding geographic area are identified, the user interface may display the identified patient cases as selectable items. Users may select all of the patient cases within the geographic area as a group for batch scheduling operations. The selection of patient cases surrounding the pin may enable users to schedule visits to multiple nearby patient cases in a single scheduling operation.

In some cases, the map interface may display visual indicators highlighting patient cases located within the selected geographic area. When a pin is dropped on the map interface, case markers corresponding to patient cases within the geographic area may be visually distinguished from case markers outside the geographic area. The visual distinction may include highlighting, color changes, selection borders, or other graphical effects that indicate which patient cases are included in the selected geographic area.

In some aspects, users may select a subset of the patient cases identified within the geographic area or may select all identified patient cases for scheduling. The user interface may provide selection controls that enable users to add or remove individual patient cases from the selection before proceeding with scheduling operations. Users may review case details for patient cases within the selected geographic area before confirming the selection for scheduling.

In some implementations, the selection of patient cases within a geographic area may enable grouped scheduling operations. After selecting patient cases surrounding a dropped pin, users may initiate a scheduling request for the selected patient cases. The scheduling request may specify a date range and time range during which the user plans to visit the geographic area containing the selected patient cases. The server may process the scheduling request to coordinate visits to the selected patient cases based on the specified date and time parameters.

In some cases, the geographic area selection functionality may assist field reimbursement managers who cover large territories. Field reimbursement managers may travel between different geographic regions within a territory, such as traveling between different cities within a state. The ability to drop a pin on the map interface and select surrounding patient cases as a group may enable field reimbursement managers to plan travel and schedule visits to multiple patient cases located in the same geographic region.

In some aspects, the server may generate a schedule grouping the identified patient cases based on proximity to each other. As described above, the server may identify patient cases located within a selected geographic area for scheduling. After identifying patient cases within the geographic area, the server may generate a schedule that groups the identified patient cases based on the geographic proximity of the patient cases to each other. The grouping of patient cases based on proximity may enable field reimbursement managers to visit multiple patient cases in a single trip to a geographic region.

In some implementations, the server may analyze the geographic coordinates of the identified patient cases to determine proximity relationships between the patient cases. The server may calculate distances between patient case locations and group patient cases that are located within a threshold distance of each other. The proximity-based grouping may reduce travel time between patient case visits by organizing visits to nearby patient cases in sequence.

In some cases, the server may generate a schedule that orders patient case visits based on geographic proximity. The schedule may arrange patient case visits such that a field reimbursement manager travels from one patient case location to a nearby patient case location rather than traveling between distant locations. The proximity-based ordering may enable efficient routing through multiple patient case locations within a geographic region.

In some aspects, field reimbursement managers may specify date ranges for visiting specific geographic areas. Field reimbursement managers may cover large territories, such as an entire state, and may travel between different geographic regions within the territory. When a field reimbursement manager plans to visit a particular geographic area, the field reimbursement manager may specify a date range during which the field reimbursement manager will be present in the geographic area. The date range may include a start date and an end date that define the period during which the field reimbursement manager plans to conduct visits in the geographic area.

In some implementations, the user interface may display a scheduling request interface that enables field reimbursement managers to specify date range parameters. The scheduling request interface may include date selection controls for specifying a start date and an end date. The scheduling request interface may also include time selection controls for specifying a time range during which the field reimbursement manager is available for visits. The date range and time range parameters may define the scheduling window for patient case visits in the selected geographic area.

In some cases, the scheduling request interface may display a calendar view that enables field reimbursement managers to visualize the specified date range. The calendar view may display days within the date range and may indicate existing scheduled appointments or commitments. Field reimbursement managers may use the calendar view to select date ranges that do not conflict with existing schedule entries.

In some aspects, field reimbursement managers may submit scheduling requests for multiple patient cases. After selecting patient cases within a geographic area and specifying a date range, the field reimbursement manager may submit a scheduling request that encompasses the selected patient cases. The scheduling request may include the identifiers of the selected patient cases, the specified date range, and the specified time range. The server may receive the scheduling request and process the scheduling request to coordinate visits to the selected patient cases.

In some implementations, the server may transmit scheduling requests to sales representatives aligned with healthcare providers associated with the selected patient cases. As described above, patient cases may be linked to healthcare providers through reference identifiers stored in patient case records. The server may retrieve healthcare provider data from a second database based on the reference identifiers and identify sales representatives aligned with the healthcare providers. The server may transmit scheduling request notifications to the identified sales representatives to coordinate joint visits between field reimbursement managers and sales representatives.

In some cases, the scheduling request may enable coordination of joint visits between field reimbursement managers and sales representatives. Field reimbursement managers may coordinate with sales representatives who have established relationships with healthcare providers associated with patient cases. The sales representatives may have knowledge of healthcare provider availability, office hours, and scheduling preferences. The scheduling request may enable sales representatives to schedule appointments with healthcare providers on behalf of field reimbursement managers based on the specified date range and time range.

In some aspects, the user interface may display a submit request control that enables field reimbursement managers to submit scheduling requests. The submit request control may be displayed on the scheduling request interface after the field reimbursement manager has selected patient cases and specified date range parameters. When the field reimbursement manager activates the submit request control, the server may receive the scheduling request and initiate the scheduling coordination process.

In some implementations, the server may generate scheduling notifications based on submitted scheduling requests. The scheduling notifications may be transmitted to sales representatives aligned with the healthcare providers associated with the selected patient cases. The scheduling notifications may include information about the patient cases, the requested date range, and the requested time range. Sales representatives may receive the scheduling notifications and respond by scheduling appointments within the requested parameters.

In some cases, the scheduling request interface may display a cancel control that enables field reimbursement managers to cancel scheduling requests before submission. The cancel control may enable field reimbursement managers to return to the map interface without submitting a scheduling request. Field reimbursement managers may use the cancel control to modify patient case selections or date range parameters before submitting a scheduling request.

In some aspects, the system may enable field reimbursement managers to schedule joint visits with pharmaceutical sales representatives aligned to specific healthcare providers. Field reimbursement managers may coordinate with sales representatives to conduct joint visits to healthcare provider offices, where the sales representatives may have established relationships with the healthcare providers. The joint visits may enable field reimbursement managers to address patient case issues while leveraging the sales representatives' familiarity with healthcare provider schedules, preferences, and office procedures.

In some implementations, the system may provide a scheduling request workflow where field reimbursement managers submit scheduling requests that are received by sales representatives. When a field reimbursement manager submits a scheduling request for patient cases associated with a healthcare provider, the server may identify sales representatives aligned with the healthcare provider. The server may transmit the scheduling request to the identified sales representatives, enabling the sales representatives to review the scheduling request and coordinate appointment scheduling with the healthcare provider.

In some cases, sales representatives may receive scheduling requests from field reimbursement managers through a scheduling interface within a client application. The scheduling interface may display pending scheduling requests submitted by field reimbursement managers, where each scheduling request may include information about the patient cases, the requested date range, and the requested time range. Sales representatives may review the scheduling requests and determine appropriate appointment times based on healthcare provider availability.

In some aspects, the system may enable sales representatives to drag and drop scheduling requests onto a calendar view. The calendar view may display the sales representative's schedule, including existing appointments and available time slots. When a sales representative receives a scheduling request from a field reimbursement manager, the sales representative may drag the scheduling request from a pending requests list and drop the scheduling request onto a time slot in the calendar view. The drag and drop interaction may create a scheduled appointment at the selected time slot.

In some implementations, the calendar view displayed to sales representatives may include a visual representation of the field reimbursement manager's availability. When a sales representative reviews a scheduling request from a field reimbursement manager, the calendar view may display the field reimbursement manager's availability as grayed out regions on the calendar. The grayed out regions may indicate time periods during which the field reimbursement manager is unavailable for joint visits. Time periods during which the field reimbursement manager is available may be displayed without the grayed out visual treatment, enabling the sales representative to identify time slots that align with the field reimbursement manager's availability.

In some cases, the grayed out availability display may be derived from the date range and time range parameters specified by the field reimbursement manager in the scheduling request. When a field reimbursement manager specifies a date range and time range for visiting a geographic area, the server may transmit the availability information to sales representatives along with the scheduling request. The sales representative's calendar view may render time periods outside the specified date range and time range as grayed out, indicating that the field reimbursement manager is not available during those time periods.

In some aspects, the calendar view may display the field reimbursement manager's availability alongside the sales representative's own schedule. The sales representative may view both the sales representative's existing appointments and the field reimbursement manager's available time periods in a single calendar view. The combined display may enable the sales representative to identify time slots that are available for both the sales representative and the field reimbursement manager.

In some implementations, when a sales representative drops a scheduling request onto a time slot in the calendar view, the server may create a scheduled appointment record. The scheduled appointment record may include the appointment date and time, the patient cases associated with the appointment, the healthcare provider to be visited, and the identifiers of the field reimbursement manager and sales representative participating in the joint visit. The server may transmit a confirmation notification to the field reimbursement manager indicating that the scheduling request has been scheduled.

In some cases, the scheduling request workflow may enable coordination between field reimbursement managers and sales representatives without requiring direct communication outside the system. Field reimbursement managers may submit scheduling requests through the system, and sales representatives may respond by scheduling appointments through the calendar interface. The system may track the status of scheduling requests and provide visibility into pending, scheduled, and completed requests.

In some aspects, the server may track a patient journey comprising a plurality of workflow stages. A patient journey may represent the progression of a patient through a patient services program from initial intake through ongoing therapy management. The plurality of workflow stages may include at least one of intake, enrollment, benefits verification, coverage verification, fulfillment, or adherence monitoring. Each workflow stage may comprise one or more tasks that are completed as the patient progresses through the patient services program.

In some implementations, the system may include configurable patient journeys that may be customized per brand. A brand may correspond to a pharmaceutical product, a therapeutic treatment, or a patient services program offered by an organization. Different brands may have different patient journey configurations based on the therapeutic area, treatment type, or organizational requirements associated with the brand. The configurable patient journeys may enable organizations to define workflow stages and tasks that align with the specific requirements of each brand.

In some cases, a patient journey may comprise multiple phases, where each phase corresponds to a workflow stage in the patient journey. Each phase may include a series of tasks that are completed in order to move the patient forward through the patient journey. The phases and tasks within each phase may be configurable based on organizational needs, therapeutic area requirements, or treatment protocols associated with the patient services program.

In some aspects, the intake workflow stage may comprise tasks associated with receiving and processing patient enrollment information. Intake tasks may include receiving patient enrollment forms, extracting patient demographic information, verifying patient identity, and creating patient records in the first database. As described above, the server may receive fax documents containing patient information, perform optical character recognition on the fax documents to extract text, and automatically populate patient intake forms with extracted data fields.

In some implementations, the enrollment workflow stage may comprise tasks associated with enrolling a patient in a patient services program. Enrollment tasks may include selecting a patient journey associated with a brand, collecting prescription information, collecting insurance information, and confirming prescriber authorization. The enrollment workflow stage may establish the patient's participation in the patient services program and may initiate subsequent workflow stages.

In some cases, the benefits verification workflow stage may comprise tasks associated with verifying a patient's insurance benefits and coverage. Benefits verification tasks may include submitting benefit verification requests to e-services providers or pharmaceutical hubs, receiving benefit verification responses, and reviewing coverage information. The benefits verification workflow stage may determine whether a patient has coverage for a prescribed treatment and may identify coverage details such as deductibles, copayments, and out-of-pocket maximums.

In some aspects, the coverage verification workflow stage may comprise tasks associated with determining coverage outcomes and managing prior authorization processes. Coverage verification tasks may include benefits verification and prior authorization, assistance program eligibility determination, and sending summary of benefits communications to healthcare providers. The coverage verification workflow stage may determine whether a patient is covered, not covered, or partially covered for a prescribed treatment, and may determine whether prior authorization is required.

In some implementations, the fulfillment workflow stage may comprise tasks associated with coordinating medication fulfillment through specialty pharmacies. Fulfillment tasks may include transmitting prescription and financial information to specialty pharmacies, tracking shipment status, and confirming medication delivery. The fulfillment workflow stage may track specialty pharmacy triage processes and may record shipment tracking information associated with medication deliveries.

In some cases, the adherence monitoring workflow stage may comprise tasks associated with monitoring patient compliance with prescribed therapy. Adherence monitoring tasks may include scheduling patient check-ins, conducting follow-up communications, and tracking patient therapy status. The adherence monitoring workflow stage may establish communication cadences based on patient preferences and may identify patients at risk of discontinuation.

In some aspects, each task within a workflow stage may be represented as a case. When a task is completed, the associated case may be marked as solved, and the task status may be updated to indicate completion. The completion of tasks within a phase may enable progression to subsequent phases in the patient journey. The server may track task completion status and may display visual representations of patient journey progress in user interfaces.

In some implementations, the patient journey may display status indicators for each workflow stage. Status indicators may include completed, in progress, or not started designations that indicate the current state of each workflow stage. The user interface may display completion dates for completed workflow stages and may display information collected during previous workflow stages.

In some cases, the configurable patient journeys may support variations based on therapeutic area or treatment type. While workflow stages may be similar across different patient journeys, specific tasks within each stage may vary based on the requirements of the therapeutic area or treatment type. The configurability of patient journeys may enable organizations to adapt workflow stages and tasks to meet the specific needs of different patient populations and treatment protocols.

In some aspects, the server may track patient journey progression and may store patient journey data in the first database. Patient journey data may include workflow stage status, task completion records, and information collected during each workflow stage. The server may retrieve patient journey data when generating user interfaces that display patient journey progress to users such as patient support specialists, benefits specialists, and field reimbursement managers.

In some aspects, the system may use Vault platform lifecycles and workflows for benefits verification and prior authorization processes. The Vault platform lifecycles may define states through which benefits verification and prior authorization tasks progress, such as pending, in progress, awaiting response, review, and complete states. The Vault platform workflows may define transitions between lifecycle states and may specify actions that occur when tasks move between states. The combination of lifecycles and workflows may provide a framework for managing benefits verification and prior authorization processes within the patient services program.

In some implementations, the benefits verification and prior authorization lifecycles and workflows may be configurable to meet organizational needs. Organizations may define custom lifecycle states, workflow transitions, and associated actions based on the requirements of the patient services program. The configurability may enable organizations to adapt the benefits verification and prior authorization processes to align with organizational policies, therapeutic area requirements, or treatment protocols. Different brands or patient journeys may have different lifecycle and workflow configurations based on the specific requirements associated with each brand or patient journey.

In some cases, the system may support integration with e-services providers or pharmaceutical hubs for outsourced benefits verification. Pharmaceutical companies may partner with e-services providers or pharmaceutical hubs that perform benefits verification on behalf of the pharmaceutical companies. The system may transmit benefits verification requests to the e-services providers or pharmaceutical hubs and may receive benefits verification responses from the e-services providers or pharmaceutical hubs. The integration may enable pharmaceutical companies to outsource benefits verification functions while maintaining visibility into the benefits verification process within the patient services system.

In some aspects, the benefits verification workflow may include tasks for confirming data to be transmitted to an e-services provider or pharmaceutical hub. Before transmitting a benefits verification request, the system may display the data to be transmitted for review and confirmation by a user. The data may include patient demographic information, insurance information, prescription details, and other information used by the e-services provider or pharmaceutical hub to perform benefits verification. Users may verify the data before the system transmits the benefits verification request.

In some implementations, the system may await responses from e-services providers or pharmaceutical hubs after transmitting benefits verification requests. The lifecycle state of a benefits verification task may transition to an awaiting response state while the system waits for a response from the e-services provider or pharmaceutical hub. When a response is received from the e-services provider or pharmaceutical hub, the lifecycle state may transition to a review state, enabling users to review the response information.

In some cases, the system may receive benefits verification responses containing coverage information. Coverage information may include coverage status indicating whether the patient is covered, not covered, or partially covered for a prescribed treatment. Coverage information may also include coverage details such as deductibles, copayments, out-of-pocket maximums, and effective dates. The system may store the received coverage information in association with the patient case record in the first database.

In some aspects, the system may receive benefits verification responses containing prior authorization information. Prior authorization information may indicate whether prior authorization is required for the prescribed treatment. When prior authorization is required, the system may initiate prior authorization workflows to manage the prior authorization process. The prior authorization workflows may include tasks for submitting prior authorization requests, tracking prior authorization status, and recording prior authorization outcomes.

In some implementations, users may review benefits verification response information and provide a coverage determination verdict. The coverage determination verdict may indicate whether the patient is covered, not covered, or partially covered based on the information received from the e-services provider or pharmaceutical hub. The system may record the coverage determination verdict in association with the patient case record and may update the lifecycle state of the benefits verification task to indicate completion.

In some cases, the patient journey view may display information surfaced from previous workflow steps. When a user navigates to the patient journey view for a patient case, the system may display information collected during previous workflow stages. The displayed information may include coverage status indicating whether the patient is covered, not covered, or partially covered. The displayed information may also include prior authorization status indicating whether prior authorization is required for the prescribed treatment.

In some aspects, the patient journey view may display coverage status and prior authorization requirements as surfaced information. The coverage status may be displayed as a visual indicator or text label indicating the coverage determination outcome. The prior authorization requirements may be displayed as a visual indicator or text label indicating whether prior authorization is required. The surfaced information may enable users to view the current coverage and prior authorization status without navigating to detailed task records.

In some implementations, the system may configure which information is surfaced on the patient journey view. Organizations may specify which data fields from previous workflow steps are displayed on the patient journey view. The configuration may enable organizations to surface information that is relevant to the patient services program and to the users who access the patient journey view. Different patient journeys may have different configurations for surfaced information based on the requirements of the associated brand or therapeutic area.

In some cases, the surfaced information on the patient journey view may be updated as workflow tasks are completed. When a benefits verification task is completed and a coverage determination verdict is recorded, the patient journey view may be updated to display the coverage status. When a prior authorization task is completed and a prior authorization outcome is recorded, the patient journey view may be updated to display the prior authorization status. The updates may enable users to view current information when accessing the patient journey view.

In some aspects, the prior authorization workflow may include tasks for managing prior authorization submissions and outcomes. Prior authorization tasks may include preparing prior authorization requests, submitting prior authorization requests to payers, tracking prior authorization status, and recording prior authorization outcomes. The prior authorization outcomes may include approved, denied, or pending designations. When a prior authorization is denied, the system may initiate appeal workflows or notify appropriate personnel to address the denial.

In some implementations, the system may track prior authorization requirements and outcomes in association with patient case records. The first database may store prior authorization data including prior authorization request dates, submission dates, payer responses, and outcome determinations. The prior authorization data may be accessible through the patient journey view and through detailed task records associated with the patient case.

In some aspects, the system may include assistance program eligibility determination based on collected patient data. As described above, the coverage verification workflow stage may include tasks for benefits verification and prior authorization, assistance program eligibility determination, and sending summary of benefits communications to healthcare providers. The assistance program eligibility determination may evaluate patient data collected during previous workflow stages to determine whether a patient qualifies for one or more patient assistance programs offered by a pharmaceutical company or patient services organization.

In some implementations, the system may determine assistance program eligibility based on patient data including insurance type, deductible levels, and prior authorization requirements. Insurance type may indicate whether a patient has commercial insurance, government insurance such as Medicare or Medicaid, or no insurance coverage. Deductible levels may indicate the amount a patient pays for covered healthcare services before an insurance plan begins to pay, including individual deductibles and family deductibles. Prior authorization requirements may indicate whether prior authorization is required for a prescribed treatment and the status of any prior authorization requests.

In some cases, the system may support multiple types of patient assistance programs. Patient assistance programs may include Patient Assistance Programs (PAP), copay assistance programs, and quick start programs. Each type of patient assistance program may have different eligibility criteria based on patient characteristics and coverage status.

In some aspects, a Patient Assistance Program (PAP) may provide medication at reduced cost or no cost to patients who meet eligibility criteria. PAP eligibility may be based on insurance type, where patients with commercial insurance may not be eligible for PAP while patients without commercial insurance or with insufficient coverage may be eligible. The system may evaluate the insurance type stored in association with a patient case record to determine PAP eligibility.

In some implementations, a copay assistance program may provide financial assistance to reduce out-of-pocket costs for patients with insurance coverage. Copay assistance eligibility may be based on deductible levels, where patients with high deductibles may be eligible for copay assistance. The system may evaluate deductible information received during benefits verification to determine whether a patient has a high deductible that qualifies the patient for copay assistance.

In some cases, a quick start program may provide medication to patients while prior authorization or other coverage processes are pending. Quick start program eligibility may be based on prior authorization requirements, where patients for whom prior authorization is required may be eligible for quick start programs to enable therapy initiation while awaiting prior authorization approval. The system may evaluate prior authorization status to determine quick start program eligibility.

In some aspects, the system may automatically determine assistance program eligibility based on values collected during previous workflow stages. When a patient case progresses through the benefits verification workflow stage, the system may collect coverage information including insurance type, deductible amounts, and prior authorization requirements. The system may use the collected values to evaluate eligibility criteria for each type of patient assistance program and determine which programs the patient may be eligible for.

In some implementations, the assistance program eligibility determination may be performed as a task within the coverage verification workflow stage. The task may evaluate patient data against eligibility criteria configured for each assistance program type. Upon completion of the assistance program eligibility task, the system may record eligibility determinations in association with the patient case record in the first database.

In some cases, the system may display assistance program eligibility information on the patient journey view. When a user navigates to the patient journey view for a patient case, the system may display eligibility status for each type of patient assistance program. The eligibility status may indicate whether the patient is eligible, not eligible, or pending determination for each assistance program type.

In some aspects, the eligibility criteria for patient assistance programs may be configurable based on organizational requirements. Different brands or patient journeys may have different assistance program configurations based on the programs offered by the pharmaceutical company or patient services organization. The configurability may enable organizations to define eligibility rules that align with the specific assistance programs available for each brand or therapeutic area.

In some implementations, the system may use the assistance program eligibility determination to identify coverage gaps and recommend appropriate assistance programs. When a patient is determined to have insufficient coverage, coverage gaps, or lack of coverage, the system may identify assistance programs for which the patient may be eligible. The identification of eligible assistance programs may enable patient services personnel to enroll patients in programs that address coverage gaps and facilitate therapy initiation.

In some aspects, the fulfillment workflow stage may comprise tasks associated with coordinating medication fulfillment through specialty pharmacies. Specialty pharmacies may handle high-cost medications that involve complex storage protocols, such as cold chain requirements, and may provide extensive monitoring and support services for patients receiving specialty medications. The system may track specialty pharmacy fulfillment including triage flows, shipment status, tracking numbers, and delivery confirmation.

In some implementations, the system may support multiple specialty pharmacy integration methods for transmitting prescription and financial information to specialty pharmacies. The integration methods may include application programming interface (API) integration, fax transmission, and Secure File Transfer Protocol (SFTP) file payload delivery. Different specialty pharmacies may support different integration methods, and the system may be configured to communicate with each specialty pharmacy using the integration method supported by the specialty pharmacy.

In some cases, the system may transmit prescription information and financial information to a specialty pharmacy using API integration. API integration may enable real-time or near-real-time communication between the system and the specialty pharmacy. The system may transmit data payloads containing prescription details, patient information, and financial information to the specialty pharmacy through API calls, and may receive response data from the specialty pharmacy through API responses.

In some aspects, the system may transmit prescription information and financial information to a specialty pharmacy using fax transmission. Fax transmission may be used when a specialty pharmacy does not support API integration or when fax is the preferred communication method for the specialty pharmacy. The system may generate fax documents containing prescription details and financial information and transmit the fax documents to the specialty pharmacy.

In some implementations, the system may transmit prescription information and financial information to a specialty pharmacy using SFTP file payload delivery. SFTP file payload delivery may involve generating data files containing prescription and financial information and depositing the data files onto an SFTP server accessible by the specialty pharmacy. The specialty pharmacy may retrieve the data files from the SFTP server and process the prescription and financial information contained within the data files.

In some cases, the system may track specialty pharmacy triage flows. Triage flows may represent the processes through which specialty pharmacies receive, process, and fulfill prescription requests. Different specialty pharmacies may have different triage flows based on the specialty pharmacy's operational procedures. The system may track the status of prescription requests as the prescription requests progress through the specialty pharmacy's triage flow.

In some aspects, the system may track shipment status associated with medication fulfillment. Shipment status may indicate whether a medication shipment has been prepared, shipped, in transit, or delivered. The system may receive shipment status updates from specialty pharmacies through the integration methods described above, including API integration, fax transmission, or SFTP file payload delivery. The system may store shipment status information in association with patient case records in the first database.

In some implementations, the system may track tracking numbers associated with medication shipments. When a specialty pharmacy ships medication to a patient, the specialty pharmacy may provide a tracking number that enables tracking of the shipment through a shipping carrier. The system may receive tracking numbers from specialty pharmacies and store the tracking numbers in association with patient case records. Users may access tracking numbers through the patient journey view or through case detail views to monitor the status of medication shipments.

In some cases, the system may track delivery confirmation associated with medication shipments. Delivery confirmation may indicate that a medication shipment has been delivered to the patient. The system may receive delivery confirmation information from specialty pharmacies or from shipping carriers. The system may update patient case records to reflect delivery confirmation and may update the fulfillment workflow stage status to indicate completion upon delivery confirmation.

In some aspects, the patient journey view may display fulfillment status information including specialty pharmacy identification, shipment status, tracking numbers, and delivery confirmation. When a user navigates to the patient journey view for a patient case, the system may display the current fulfillment status and may display information collected during the fulfillment workflow stage. The displayed information may enable users to monitor the progress of medication fulfillment and to identify cases where fulfillment issues have arisen.

In some implementations, the system may generate cases when fulfillment issues arise. Fulfillment issues may include shipment delays, missing shipments, or delivery failures. When the system detects a fulfillment issue based on shipment status information received from a specialty pharmacy, the system may create a case record associated with the fulfillment issue. The case record may be assigned to appropriate personnel for resolution, such as patient support specialists or field reimbursement managers.

In some cases, the fulfillment workflow stage may include tasks for confirming receipt of medication by the patient. Confirm receipt tasks may involve contacting the patient to verify that the patient has received the medication shipment. The system may track confirm receipt task status and may update the fulfillment workflow stage status based on task completion. Upon confirmation of receipt, the patient journey may progress to subsequent workflow stages such as adherence monitoring.

In some aspects, the adherence monitoring workflow stage may comprise tasks associated with monitoring patient compliance with prescribed therapy and maintaining patient engagement throughout the treatment period. The system may track patient adherence status and may identify patients who may be at risk of discontinuing therapy. Adherence monitoring tasks may include scheduling patient check-ins, conducting follow-up communications, and tracking patient therapy status over time.

In some implementations, the system may include adherence monitoring with a configurable check-in cadence based on patient communication preferences. The check-in cadence may define the frequency and timing of follow-up communications with a patient to verify that the patient remains compliant with prescribed therapy. The check-in cadence may be configured based on patient preferences, therapeutic area requirements, or organizational policies associated with the patient services program.

In some cases, the system may store patient communication preferences indicating how a patient prefers to receive communications. Patient communication preferences may include phone calls, email, or text messaging (SMS). When scheduling adherence check-ins, the system may use the stored patient communication preferences to determine the communication channel through which to conduct the check-in. The system may schedule check-in tasks based on the configured cadence and may assign the check-in tasks to patient support specialists or other personnel responsible for patient communications.

In some aspects, the configurable check-in cadence may enable organizations to define different check-in frequencies for different patient populations or therapeutic areas. Some patients may receive weekly check-ins while other patients may receive monthly check-ins based on the nature of the prescribed therapy, the patient's therapy status, or other factors. The system may store cadence configuration settings in association with patient journey configurations, enabling different patient journeys to have different check-in cadence settings.

In some implementations, the system may track the date of the next scheduled check-in for each patient. When a check-in is completed, the system may calculate the date of the next check-in based on the configured cadence and may schedule a subsequent check-in task. The system may display upcoming check-in dates on patient journey views and on user dashboards, enabling patient support specialists to view scheduled check-ins and plan communications accordingly.

In some cases, the system may support multiple communication channels for adherence check-ins. Communication channels may include phone calls, email communications, and text messaging (SMS). The system may be configured to support additional communication channels as the channels become available. When conducting a check-in, the system may initiate communication through the patient's preferred communication channel and may record the outcome of the check-in in association with the patient case record.

In some aspects, the system may display patients at risk of discontinuation with information including prescriber and date of last fill. The system may identify patients who may be at risk of discontinuing therapy based on adherence indicators, fill history, or other factors. Patients identified as at risk of discontinuation may be displayed in a dedicated section of a user dashboard, enabling patient services personnel to prioritize outreach to patients who may discontinue therapy.

In some implementations, the system may display a patients at risk of discontinuation list on a field reimbursement manager home dashboard or other user interface. The patients at risk of discontinuation list may display patient names, prescriber names, and date of last fill for each patient identified as at risk. The date of last fill may indicate when the patient last received a medication fill from a specialty pharmacy, enabling users to assess how long the patient has been without medication and to prioritize outreach accordingly.

In some cases, the prescriber information displayed in the patients at risk of discontinuation list may be retrieved from the second database based on healthcare provider reference identifiers stored in patient case records. The system may retrieve prescriber names and other healthcare provider data from the second database and display the prescriber information alongside patient information in the patients at risk of discontinuation list. The display of prescriber information may enable field reimbursement managers to coordinate with healthcare providers regarding patients at risk of discontinuation.

In some aspects, the system may generate cases when patients are identified as at risk of discontinuation. The cases may be assigned to appropriate personnel, such as patient support specialists or field reimbursement managers, for follow-up and intervention. The cases may include information about the patient's adherence status, date of last fill, and prescriber information to facilitate outreach and resolution.

In some implementations, the patients at risk of discontinuation list may include navigation controls that enable users to view additional patients or to access detailed patient records. Users may select a patient from the patients at risk of discontinuation list to navigate to the patient's case record or patient journey view. The navigation may enable users to review patient details and to initiate communications or other interventions to address discontinuation risk.

In some cases, the system may track discontinuation risk indicators based on fill history and adherence data. The system may calculate the expected date of the next fill based on prescription details including dosage and number of refills. When a patient's expected fill date passes without a recorded fill, the system may flag the patient as at risk of discontinuation. The system may update discontinuation risk status as new fill data is received from specialty pharmacies or other sources.

In some aspects, the adherence monitoring workflow stage may track patient therapy status including on therapy, off therapy, and discontinuation risk designations. The patient therapy status may be displayed on patient journey views and may be updated as adherence check-ins are completed and as fill data is received. The therapy status designations may enable patient services personnel to monitor patient adherence across a patient population and to identify patients who may benefit from additional support or intervention.

In some aspects, the system may support multiple communication channels for patient engagement and outbound communications. Communication channels supported by the system may include fax, email, phone, and SMS text messaging. The system may enable patient services personnel to communicate with patients and healthcare providers through the communication channel appropriate for each communication type and recipient preference.

In some implementations, fax may serve as a communication channel for both inbound and outbound communications. As described above, the system may receive inbound fax documents containing patient enrollment forms and other patient-related documents. The system may also transmit outbound fax communications to healthcare providers, such as summary of benefits documents transmitted to prescribers during the coverage verification workflow stage.

In some cases, the system may include approved fax templates for outbound fax communications. Approved fax templates may be pre-approved document templates that define the structure and content of outbound fax communications. The approved fax templates may be stored in a controlled content repository and may be subject to approval workflows that ensure the templates comply with organizational policies and regulatory requirements.

In some aspects, approved fax templates may contain tokens that are automatically populated with verified patient journey data. Tokens may be placeholder elements within the fax template that are replaced with actual data values when the fax communication is generated. The tokens may reference data fields collected during various workflow stages of the patient journey, including patient demographic information, coverage information, benefits verification results, and other data associated with the patient case.

In some implementations, when a user initiates an outbound fax communication, the system may retrieve data values associated with the patient case and populate the tokens in the selected fax template with the retrieved data values. The data values used to populate the tokens may be verified data that has been collected and confirmed during previous workflow stages. The system may display the populated fax template to the user for review before transmission, enabling the user to verify that the data values have been correctly populated.

In some cases, the system may populate fax template tokens with coverage details collected during the benefits verification workflow stage. Coverage details may include group number, effective date, individual deductible amounts, family deductible amounts, and out-of-pocket maximum amounts. The system may retrieve the coverage details from the patient case record and insert the coverage details into corresponding token locations within the fax template.

In some aspects, email may serve as a communication channel for patient engagement and healthcare provider communications. The system may transmit email communications to patients and healthcare providers based on communication preferences and workflow requirements. Email communications may include appointment reminders, adherence check-in messages, and other patient engagement communications.

In some implementations, the system may determine an email domain for outbound email communications based on the geographic location of the recipient. The geographic location may be determined based on address information associated with the recipient, such as country of residence. The system may select an email domain that corresponds to the recipient's geographic location, enabling branded email communications that use country-specific domain extensions.

In some cases, a recipient located in a first country may receive an email communication from a first branded domain corresponding to the first country, while a recipient located in a second country may receive an email communication from a second branded domain corresponding to the second country. For example, a recipient in Spain may receive an email from a domain with a Spanish country code extension, while a recipient in Germany may receive an email from a domain with a German country code extension. The geographic-based email domain determination may enable organizations to present localized branding in email communications.

In some aspects, phone communications may serve as a communication channel for patient engagement. The system may support phone calls between patient services personnel and patients for adherence check-ins, enrollment communications, and other patient interactions. The system may track phone communication outcomes in association with patient case records.

In some implementations, SMS text messaging may serve as a communication channel for patient engagement. The system may transmit text messages to patients based on patient communication preferences. Text messaging may be used for adherence reminders, appointment notifications, and other patient communications. The system may store patient communication preferences indicating whether a patient prefers to receive communications via phone, email, or text messaging.

In some cases, the system may select a communication channel for patient communications based on stored patient communication preferences. When scheduling adherence check-ins or other patient communications, the system may retrieve the patient's preferred communication channel from the patient record and initiate communication through the preferred channel. The system may record the outcome of communications in association with the patient case record regardless of the communication channel used.

In some aspects, the system may support integration with Amazon Connect for cloud-based contact center services. Amazon Connect is a cloud-based contact center service that provides telephony infrastructure and call management capabilities. The integration with Amazon Connect may enable outbound calling directly from the system interface, allowing patient services personnel to initiate phone calls to patients and healthcare providers without leaving the patient CRM application.

In some implementations, the system may include an omni-channel connector that interfaces with Amazon Connect to provide telephony functionality within the system. The omni-channel connector may establish communication between the patient CRM system and the Amazon Connect contact center infrastructure. The omni-channel connector may handle call initiation requests, call routing, call status updates, and call termination events.

In some cases, the omni-channel connector may enable click-to-dial functionality when phone numbers are displayed on screen. Click-to-dial functionality may allow users to initiate a phone call by selecting a phone number displayed in the user interface. When a user selects a phone number displayed on a patient record, a case detail view, or another user interface screen, the system may transmit a call initiation request to Amazon Connect through the omni-channel connector. Amazon Connect may establish the outbound call and connect the user to the dialed phone number.

In some aspects, phone numbers displayed in the user interface may be rendered as selectable elements that trigger click-to-dial functionality. The user interface may display phone numbers associated with patients, healthcare providers, or other contacts as hyperlinks or buttons that users may select to initiate calls. When a user selects a phone number element, the system may invoke the omni-channel connector to initiate an outbound call through Amazon Connect.

In some implementations, the click-to-dial functionality may be available across multiple user interface screens within the patient CRM application. Phone numbers displayed on patient overview screens, case detail views, patient intake forms, and healthcare provider contact panels may support click-to-dial functionality. The consistent availability of click-to-dial functionality across user interface screens may enable users to initiate calls from the context in which the user is working without navigating to a separate dialing interface.

In some cases, the system may display call status information when a call is initiated through the click-to-dial functionality. Call status information may include call connection status, call duration, and call participant information. The user interface may display an active call indicator when a call is in progress, enabling users to view call status while continuing to access patient data and case information in the user interface.

In some aspects, the system may record call metadata in association with patient case records when calls are initiated through the omni-channel connector. Call metadata may include call start time, call end time, call duration, and the phone number dialed. The call metadata may be stored in the first database in association with the patient case record, enabling tracking of phone communications as part of the patient case history.

In some implementations, the integration with Amazon Connect may support inbound call handling in addition to outbound calling. When an inbound call is received through Amazon Connect, the system may identify the caller based on the caller's phone number and may display patient or healthcare provider information associated with the caller. The omni-channel connector may route inbound calls to appropriate patient services personnel based on call routing rules configured within Amazon Connect.

In some cases, the omni-channel connector may support call transfer and conference call functionality. Users may transfer active calls to other patient services personnel or may add additional participants to a call. The call transfer and conference call functionality may be accessible through controls displayed in the user interface during an active call.

In some aspects, the system may include case notes functionality for capturing notes during patient conversations. The case notes functionality may enable users to record information obtained during phone calls, meetings, or other interactions with patients. Users may enter case notes in a text entry field displayed in the user interface while a conversation is in progress or after a conversation concludes. The case notes may be stored in association with the patient case record in the first database, enabling other users who access the patient case to view notes captured during previous interactions.

In some implementations, the user interface may include a case note tab or panel that provides access to case notes functionality. The case note tab may be displayed alongside other case-related tabs such as case messaging, case timeline, and guide tabs. Users may select the case note tab to access a text entry area for entering notes related to the patient case. The case notes may capture information such as patient responses, action items, follow-up requirements, and other details discussed during patient interactions.

In some cases, users may capture case notes while conducting phone calls with patients through the integrated telephony functionality described above. When a user initiates a phone call to a patient using click-to-dial functionality, the user interface may display the case note entry area alongside call status information. Users may enter notes in real-time during the phone conversation without navigating away from the active call interface. The case notes captured during the call may be associated with the patient case record and may include a timestamp indicating when the notes were entered.

In some aspects, the system may include functionality for marking cases as solved with comments. When a user completes work on a patient case, the user may change the case state to solved to indicate that the case has been resolved. The user interface may display a control for changing the case state to solved, such as a button or menu option labeled to indicate case resolution. When the user activates the control to mark the case as solved, the system may prompt the user to provide comments describing the resolution or outcome of the case.

In some implementations, the user interface may display a comment entry field when a user initiates the action to mark a case as solved. The comment entry field may enable the user to enter text describing the resolution of the case, the outcome of patient interactions, or other information relevant to the case closure. The comment entry field may include a character count indicator that displays the number of characters remaining or the number of characters entered. Users may enter comments and then activate a control to confirm the case resolution, such as a button labeled to indicate marking the case as solved.

In some cases, the comments provided when marking a case as solved may be stored in association with the patient case record. The comments may be displayed in a case timeline or case history view, enabling other users to review the resolution details when accessing the patient case record. The case timeline may display a chronological record of case activities including case state changes, comments, case reassignments, and other events associated with the patient case.

In some aspects, the system may inherit case management capabilities from a service center application. The patient CRM system may be part of an application family that includes a service center application providing case management functionality. The case management capabilities inherited from the service center application may include case creation, case assignment, case routing, case state management, and case resolution workflows. The inherited case management capabilities may enable the patient CRM system to leverage established case handling functionality while extending the functionality for patient services use cases.

In some implementations, the case management capabilities may include case inbox functionality that organizes cases for user access and processing. The case inbox may include a my cases view that displays cases assigned to the current user. The case inbox may also include channel-specific inboxes that organize cases based on the communication channel through which the case originated, such as fax inboxes and email inboxes. Users may navigate between different inbox views to access cases based on assignment or communication channel.

In some cases, the case management capabilities may include case state management functionality. Cases may progress through multiple states during the case lifecycle, such as open, in progress, escalated, and solved states. The system may track case state transitions and may record state change events in the case timeline. Users may change case states based on case progress and may provide comments when transitioning cases between states.

In some aspects, the case management capabilities may include case assignment and reassignment functionality. Cases may be assigned to users based on case type, user role, territory assignment, or queue configuration. Users may reassign cases to other users when case handling responsibilities change or when cases are escalated. Case reassignment events may be recorded in the case timeline, enabling tracking of case ownership changes throughout the case lifecycle.

In some implementations, the case management capabilities may support case severity levels that indicate the priority or urgency of cases. Case severity levels may include designations such as normal and escalated. Users may change case severity levels based on case circumstances, and severity level changes may be recorded in the case timeline. The case inbox may display case severity indicators, enabling users to identify cases that may warrant prioritized attention.

In some cases, the case management capabilities may enable multiple user types to access case information. Field reimbursement managers may access cases to resolve patient-related issues that arise during the patient journey. Call center personnel may access cases to perform patient intake and enrollment tasks. Benefits specialists may access cases to manage coverage verification workflows. The case management capabilities may provide different levels of access based on user roles, where some users may have read-only access to cases while other users may have the ability to update cases and mark cases as solved.

In some aspects, the system may include a care team assignment feature for designating personnel responsible for moving patient journeys forward. The care team assignment feature may enable organizations to assign users to patient cases based on assignment rules or through manual assignment. The care team may comprise multiple users with different roles who collaborate to support a patient through the patient journey workflow stages.

In some implementations, the system may assign care team members to patient cases based on assignment rules. Assignment rules may define criteria for automatically assigning users to patient cases based on case attributes, patient attributes, user roles, territory assignments, or other factors. When a patient case is created or when case attributes change, the system may evaluate the assignment rules and assign appropriate users to the care team for the patient case. The assignment rules may be configurable based on organizational requirements and may vary across different patient journeys or brands.

In some cases, the system may support manual assignment of care team members to patient cases. Users with appropriate permissions may add care team members to a patient case through a user interface control. The user interface may display an add member control that enables users to select additional personnel to add to the care team. Manual assignment may enable users to add care team members when assignment rules do not automatically assign all personnel involved in a patient case or when case circumstances warrant additional team members.

In some aspects, the care team may include users with different roles who perform different functions within the patient journey. Care team roles may include patient support specialists, benefits specialists, and field reimbursement managers. Patient support specialists may handle patient-facing communications, including conducting welcome calls, responding to patient inquiries, and resolving patient-facing issues that arise during the patient journey. Benefits specialists may manage coverage verification workflows, including benefits verification tasks, prior authorization processes, and assistance program eligibility determinations. Field reimbursement managers may coordinate with healthcare providers to resolve issues that arise during the patient journey, such as issues related to prior authorization submissions or healthcare provider documentation.

In some implementations, the system may display the care team on patient records. The user interface may include a care team section or panel that displays the care team members assigned to a patient case. The care team section may display the name of each care team member, the role of each care team member, and an indication of which care team member is the owner of the patient case. The care team owner may be the user with primary responsibility for the patient case.

In some cases, the care team section may be displayed on a patient overview screen alongside patient demographic information, patient journey information, and other patient-related data. The care team section may enable users to view the personnel assigned to a patient case without navigating to a separate view or screen. The display of the care team on the patient overview screen may enable users to identify the appropriate personnel to contact regarding a patient case.

In some aspects, the care team section may include navigation controls that enable users to access additional information about care team members. The user interface may display a view record control or similar navigation element adjacent to care team member entries. Users may select the view record control to navigate to a detailed record for the care team member, where the detailed record may include contact information and other user attributes.

In some implementations, the care team assignment may be updated as the patient journey progresses. When a patient case transitions between workflow stages, the system may evaluate assignment rules and update the care team membership based on the current workflow stage. For example, a benefits specialist may be added to the care team when a patient case enters the benefits verification workflow stage. The care team membership may reflect the personnel involved in the current phase of the patient journey.

In some cases, the system may track care team membership changes in association with patient case records. When care team members are added or removed from a patient case, the system may record the change in a case timeline or audit trail. The tracking of care team membership changes may enable organizations to review the personnel involved in a patient case throughout the patient journey.

In some aspects, the system may provide a patient overview screen that consolidates patient-related information on a single screen. The patient overview screen may display demographic information, insurance details, prescription information, and patient journey status in a unified view. The consolidation of patient-related information on a single screen may enable users to access patient data without navigating between multiple views or screens.

In some implementations, the patient overview screen may display patient demographic information in a designated region of the screen. Demographic information displayed on the patient overview screen may include patient name, age range, gender, geographic location, mobile phone number, email address, date of birth, preferred communication method, and physical address. The demographic information may be retrieved from a first database storing patient data including protected health information or personally identifiable information.

In some cases, the patient overview screen may display insurance details associated with the patient. Insurance details may include insurance provider name, policy information, group number, and coverage status. The insurance details may be displayed in a collapsible or expandable section on the patient overview screen, enabling users to view insurance information when relevant while conserving screen space when the information is not actively being reviewed.

In some aspects, the patient overview screen may display prescription information associated with the patient. Prescription information may include medication name, dosage, number of refills, and prescriber details. The prescription information may be displayed in a designated section of the patient overview screen alongside other patient-related data. The prescriber details displayed on the patient overview screen may include prescriber name, healthcare facility, office phone number, office fax number, office email address, physical address, national provider identifier (NPI), and date signed.

In some implementations, the patient overview screen may display patient journey status information. The patient journey status may indicate the current workflow stage of the patient within a patient services program. Workflow stages may include intake, enrollment, benefits verification, coverage verification, fulfillment, and adherence monitoring. The patient overview screen may display visual indicators for each workflow stage, where the visual indicators may indicate whether each workflow stage is completed, in progress, or not started. Completion dates may be displayed for completed workflow stages.

In some cases, the patient overview screen may display the care team assigned to the patient case. The care team section may display the names and roles of care team members, including patient support specialists, benefits specialists, and field reimbursement managers. The care team section may indicate which care team member is the owner of the patient case. The display of the care team on the patient overview screen may enable users to identify personnel responsible for the patient case without navigating to a separate view.

In some aspects, the patient overview screen may display visual indicators adjacent to fields containing protected health information or personally identifiable information. The visual indicators may comprise shield icons or borders displayed around fields designated as containing protected health information or personally identifiable information. The visual indicators may enable users to identify fields that are subject to additional security handling, including encryption at rest, encryption during transmission, and masking in audit trails.

In some implementations, the system may include consent information management functionality. The patient overview screen may display a consent information section that provides access to consent records associated with the patient. The consent information section may display an update timestamp indicating when consent records were last updated. The consent information section may include a control for viewing or updating consent records, such as a link or button labeled to indicate viewing or updating consent records.

In some cases, users may select the control for viewing or updating consent records to access detailed consent record information. The consent records may include records of patient consent for participation in patient services programs, consent for communications, consent for data sharing, and other consent types associated with the patient services program. Users may review existing consent records and may update consent records when patient consent status changes.

In some aspects, the consent information management functionality may track consent record changes over time. When a consent record is updated, the system may record the update in association with the patient record and may update the timestamp displayed in the consent information section. The tracking of consent record changes may enable organizations to maintain records of patient consent status throughout the patient journey.

In some implementations, the patient overview screen may provide navigation controls that enable users to access additional patient-related views. The patient overview screen may include a control for viewing the patient journey in detail, such as a link or button labeled to indicate viewing the patient journey. Users may select the navigation control to access a detailed patient journey view that displays workflow stages, tasks, and task completion status.

In some cases, the patient overview screen may be accessible to multiple user types based on access permissions. User types that may access the patient overview screen may include call center personnel, patient support specialists, benefits specialists, and field reimbursement managers. The system may apply access controls based on user roles to determine which users may view patient data displayed on the patient overview screen.

In some aspects, the system may include an overview dashboard that summarizes program information and performance metrics. The overview dashboard may be powered by data pages that retrieve and display data from the patient CRM system. The data pages may query patient data, case data, and workflow data stored in the first database to generate summary information and metrics for display on the overview dashboard.

In some implementations, the overview dashboard may display information that enables users to assess daily workload and case distribution. The overview dashboard may display case counts indicating the number of open cases, scheduled cases, and total cases associated with a user or territory. The overview dashboard may also display patient counts indicating the number of new patients and total patients within a user's assigned territory or program.

In some cases, the overview dashboard may display a case overview section that provides visibility into case status across a user's assigned cases. The case overview section may display counts of cases in different states, such as open cases awaiting action and scheduled cases with planned follow-up activities. The case counts displayed in the case overview section may enable users to assess the volume of work associated with the user's case assignments.

In some aspects, the overview dashboard may display today's scheduled cases in a list format. The today's scheduled cases list may display case information including case type, patient name, and prescriber name for each case scheduled for the current day. Case types displayed in the today's scheduled cases list may include prior authorization denied, shipment delayed, benefits verification not covered, and other case types that arise during the patient journey. The display of today's scheduled cases may enable users to view planned activities for the current day.

In some implementations, the overview dashboard may display a patients at risk of discontinuation section. The patients at risk of discontinuation section may display patient names, prescriber names, and date of last fill for patients identified as at risk of discontinuing therapy. The display of patients at risk of discontinuation may enable users to identify patients who may benefit from outreach or intervention.

In some cases, the overview dashboard may display product information associated with the patient services program. Product information may include product name and product identifier for pharmaceutical products associated with the patient services program. The product information may provide context for the case and patient metrics displayed on the overview dashboard.

In some aspects, the overview dashboard may include navigation controls that enable users to access additional views and detailed information. The overview dashboard may include a view schedule control that enables users to navigate to a schedule view displaying scheduled appointments and activities. The overview dashboard may also include view all controls adjacent to list sections that enable users to navigate to expanded views displaying additional items beyond those shown in the summary lists.

In some implementations, the overview dashboard may be accessible to different user types based on user roles and access permissions. Field reimbursement managers may access an overview dashboard that displays case and patient information associated with the field reimbursement manager's assigned territory. Call center personnel may access an overview dashboard that displays case and patient information associated with the call center user's assigned queues or case assignments. The overview dashboard may display different information based on the user type and access permissions of the user accessing the dashboard.

In some cases, the overview dashboard may display performance metrics that enable users and managers to assess program performance. Performance metrics may include case resolution rates, patient enrollment counts, workflow stage completion rates, and other metrics derived from data stored in the first database. The performance metrics may be calculated based on data retrieved through data pages and displayed in designated sections of the overview dashboard.

In some aspects, the overview dashboard may provide a consolidated view of program status that enables users to assess the current situation without navigating to multiple views or screens. The consolidation of program information, case counts, patient counts, scheduled activities, and performance metrics on a single dashboard may enable users to obtain an overview of program status and identify areas that may warrant attention.

In some aspects, the system may include interactions between a first database, a second database, a server, user computing devices, and various processing components to perform patient CRM functions while maintaining database segregation for regulatory compliance. The first database may store patient data including protected health information (PHI) or personally identifiable information (PII) associated with a plurality of patients. The second database may store customer relationship management data associated with a plurality of healthcare providers. The server may be in communication with the first database and the second database and may coordinate data retrieval and processing operations across the databases.

In some implementations, the data flow for patient intake may begin when a fax document containing patient information is received by the system. A fax processing subsystem may convert received fax transmissions into digital document images for storage and processing. The server may receive the fax document and transmit the fax document image to an optical character recognition (OCR) processing component. The OCR processing component may employ a multimodal large language model with vision capabilities to extract text from the fax document image. The extracted text may be transmitted back to the server for further processing.

In some cases, the server may analyze the extracted text to identify a plurality of data fields corresponding to patient information contained within the fax document. The server may recognize field labels and associated values within the extracted text using pattern matching, natural language processing, or machine learning techniques. The server may map each identified data field to a corresponding input field in a patient intake form and may automatically populate the patient intake form with the extracted data fields. The server may transmit the populated patient intake form to a user computing device for display alongside the fax document image.

In some aspects, the server may maintain a mapping between fields in the patient intake form and corresponding source locations in the fax document. When a user computing device transmits a selection of a field in the patient intake form to the server, the server may retrieve the source location associated with the selected field from the mapping. The server may generate a visual indication at the retrieved source location on the fax document and may transmit the visual indication to the user computing device for display. The user computing device may render the visual indication on the fax document image, enabling the user to verify that the auto-populated value corresponds to the correct source location in the original fax document.

In some implementations, the server may perform a patient search before creating new patient records. The server may receive search criteria from a user computing device, where the search criteria may include patient identifying attributes such as patient name, phone number, and date of birth. The server may query the first database to locate patient records matching the search criteria. The server may transmit search results to the user computing device indicating whether matching patient records were found. When no matching patient records are found, the server may create a new patient record in the first database based on data extracted from the fax document.

In some cases, when a patient record is created or updated in the first database, the server may establish associations between the patient record and healthcare providers. The server may extract healthcare provider identifying information from the fax document, such as prescriber name, national provider identifier (NPI), or healthcare facility name. The server may store a reference identifier in the patient case record within the first database, where the reference identifier corresponds to a healthcare provider record in the second database. The reference identifier may enable cross-database queries without commingling patient data with healthcare provider data.

In some aspects, when a user computing device transmits a request to access data associated with a patient case, the server may perform cross-database retrieval operations. The server may first query the first database using a case identifier or patient identifier included in the request to retrieve patient data associated with the patient case. The server may extract the healthcare provider reference identifier from the retrieved patient case data. The server may then query the second database using the healthcare provider reference identifier to retrieve healthcare provider data associated with the healthcare provider linked to the patient case.

In some implementations, the server may combine the patient data retrieved from the first database with the healthcare provider data retrieved from the second database for presentation in a user interface. The server may assemble data elements from both databases and may generate a user interface displaying the combined data. The server may transmit the user interface to the user computing device for rendering. The server may perform the combination at query time without persistently storing the combined data in either database, thereby maintaining the separation between the first database and the second database.

In some cases, the server may transmit patient data to the user computing device for display without storing the patient data locally on the user computing device. The user computing device may render the patient data in the user interface while the patient data remains stored in the first database. When the user navigates away from a view displaying patient data, the patient data may not remain stored on the user computing device. The server may retrieve patient data from the first database in real-time in response to user navigation within a client application running on the user computing device.

In some aspects, the server may apply security controls during data retrieval and transmission operations. When the server receives a request to access patient data, the server may verify that the user associated with the user computing device has authorization to access the requested patient data. The verification may include determining whether the user has access to objects containing patient data and whether the user has access to the specific patient records identified in the request. The server may apply record-level access controls based on user roles, territory assignments, or other access criteria.

In some implementations, data associated with fields designated as containing protected health information or personally identifiable information may be encrypted during transmission between the server and user computing devices. The server may encrypt patient data before transmitting the patient data to the user computing device. The user computing device may decrypt the received patient data for display in the user interface. Data stored in fields designated as containing protected health information or personally identifiable information may also be encrypted at rest within the first database.

In some cases, the server may track patient journey progression through a plurality of workflow stages. The workflow stages may include intake, enrollment, benefits verification, coverage verification, fulfillment, and adherence monitoring. As tasks within each workflow stage are completed, the server may update patient journey data stored in the first database. The server may retrieve patient journey data when generating user interfaces that display patient journey progress to users.

In some aspects, the server may coordinate data flow with external systems during the benefits verification workflow stage. The server may transmit benefits verification requests to e-services providers or pharmaceutical hubs through application programming interface (API) calls. The server may receive benefits verification responses from the e-services providers or pharmaceutical hubs containing coverage information and prior authorization requirements. The server may store the received coverage information in association with the patient case record in the first database.

In some implementations, the server may coordinate data flow with specialty pharmacies during the fulfillment workflow stage. The server may transmit prescription information and financial information to specialty pharmacies using API integration, fax transmission, or Secure File Transfer Protocol (SFTP) file payload delivery. The server may receive shipment status updates, tracking numbers, and delivery confirmation information from specialty pharmacies. The server may store the received fulfillment information in association with the patient case record in the first database.

In some cases, the server may support multiple communication channels for patient engagement. The server may interface with a cloud-based contact center service to provide telephony functionality. When a user computing device transmits a call initiation request, the server may communicate with the contact center service to establish an outbound call. The server may record call metadata in association with patient case records in the first database.

In some aspects, the server may generate outbound fax communications using approved fax templates. The server may retrieve data values associated with a patient case from the first database and may populate tokens in a selected fax template with the retrieved data values. The server may transmit the populated fax document to a healthcare provider fax number retrieved from the second database.

In some implementations, the server may coordinate scheduling operations between field reimbursement managers and sales representatives. When a field reimbursement manager submits a scheduling request for patient cases, the server may retrieve healthcare provider data from the second database based on reference identifiers stored in the patient case records. The server may identify sales representatives aligned with the healthcare providers and may transmit scheduling request notifications to the identified sales representatives. The server may track scheduling request status and may update patient case records when appointments are scheduled.

In some cases, the server may generate geographic visualizations of patient case locations. The server may retrieve geographic location data associated with patient cases from the first database. The server may generate a map interface depicting locations associated with the patient cases and may transmit the map interface to a user computing device for display. When the user computing device transmits a selection of a geographic area on the map interface, the server may identify patient cases located within the selected geographic area and may generate a schedule grouping the identified patient cases based on proximity to each other.

In some aspects, the server may maintain audit trails that track communications and data changes. The server may record the content of communications, metadata about the communications, and information regarding content accessed by recipients. When changes are made to fields containing protected health information or personally identifiable information, the server may record that a change occurred in the audit trail without revealing the actual values that were changed. The audit trail may be stored in a controlled content repository accessible for regulatory compliance purposes.

In some implementations, the server may retrieve data from the second database to maintain current healthcare provider information. The second database may communicate with multiple external sources through API interfaces to update licensing information, areas of practice, and location information for healthcare provider accounts. When the server retrieves healthcare provider data in response to a request, the server may retrieve current healthcare provider data that reflects updates received from external sources.

In some cases, the data flow may support multiple user types with different access permissions. Call center personnel may access patient data through the server to perform patient intake and enrollment tasks. Benefits specialists may access patient data and coverage information through the server to manage coverage verification workflows. Field reimbursement managers may access patient data from the first database and healthcare provider data from the second database through the server to coordinate patient services and healthcare provider interactions. The server may generate user interfaces tailored to the access permissions and role-based requirements of each user type while maintaining the patient data in the first database separate from the second database.

While various embodiments have been described above, it should be understood that they have been presented by way of example only, and not limitation. Thus, the breadth and scope of a preferred embodiment should not be limited by any of the above described exemplary embodiments, but should be defined only in accordance with the claims and their equivalents for any patent that issues claiming priority from the present provisional patent application.

In all descriptions of “servers” or other computing devices herein, whether or not the illustrations of those servers or other computing devices similarly show a server-like illustration in the figures, it should be understood that any such described servers or computing devices will similarly per form their described functions in accordance with computer readable instructions stored on a computer-readable media that are connected thereto.

Resources may encompass any types of resources for running instances including hardware (such as servers, clients, mainframe computers, networks, network storage, data sources, memory, central processing unit time, Scientific instruments, and other computing devices), as well as software, software licenses, available network services, and other non-hardware resources, or a combination thereof.

A networked computing environment may include, but is not limited to, computing grid systems, distributed computing environments, cloud computing environment, etc. Such networked computing environments include hardware and Software infrastructures configured to form a virtual organization comprised of multiple resources which may be in geographically disperse locations.

The approved content may be in any format, e.g., text, audio, video, picture, multimedia, or PDF.

Various terms used herein have special meanings within the present technical field. Whether a particular term should be construed as such a “term of art, depends on the context in which that term is used. “Connected to,” “in communication with or other similar terms should generally be construed broadly to include situations both where communications and connections are direct between referenced elements or through one or more intermediaries between the referenced elements, including through the Internet or some other communicating network. “Network,” “system,” “environment,” and other similar terms generally refer to networked computing systems that embody one or more aspects of the present disclosure. These and other terms are to be construed in light of the context in which they are used in the present disclosure and as those terms would be understood by one of ordinary skill in the art would understand those terms in the disclosed context. The above definitions are not exclusive of other meanings that might be imparted to those terms based on the disclosed context.

Words of comparison, measurement, and timing such as “at the time.” “equivalent,” “during,” “complete,” and the like should be understood to mean “substantially at the time.” “substantially equivalent,” “substantially during,” “substantially complete,” etc., where “substantially” means that such comparisons, measurements, and timings are practicable to accomplish the implicitly or expressly stated desired result.

The steps and/or operations described above in relation to an embodiment of the present disclosure may occur in a different order, or in parallel, or concurrently for different epochs, etc. depending on the specific embodiment and/or implementation, as would be understood by one of ordinary skill in the art. Different embodiments may perform actions in a different order or by different ways or means. As would be understood by one of ordinary skill in the art, some drawings are simplified representations of the actions performed, their descriptions herein simplified overviews, and real-world implementations would be much more complex, require more stages and/or components, and would also vary depending on the requirements of the particular implementation. Being simplified representations, these drawings do not show other required steps as these may be known and understood by one of ordinary skill in the art and may not be pertinent and/or helpful to the present description.

Similarly, some drawings are simplified block diagrams showing only pertinent components, and some of these components merely represent a function and/or operation well-known in the field, rather than an actual piece of hardware, as would be understood by one of ordinary skill in the art. In such cases, some or all of the components/modules may be implemented or provided in a variety and/or combinations of manners, such as at least partially firmware and/or hardware, including, but not limited to one or more application-specific integrated circuits (“ASICS”), standard integrated circuits, controllers executing appropriate instructions, and including microcontrollers and/or embedded controllers, field-programmable gate arrays (“FPGAs”), complex programmable logic devices (“CPLDs”), and the like. Some or all of the system components and/or data structures may also be stored as contents (e.g., as executable or other machine-readable software instructions or structured data) on a non-transitory computer-readable medium (e.g., as a hard disk; a memory; a computer network or cellular wireless network or other data transmission medium; or a portable media article to be read by an appropriate drive or via an appropriate connection, such as a DVD or flash memory device) so as to enable or configure the computer-readable medium and/or one or more associated computing systems or devices to execute or otherwise use or provide the contents to perform at least some of the described techniques.

One or more processors, simple micro controllers, controllers, and the like, whether alone or in a multi-processing arrangement, may be employed to execute sequences of instructions stored on non-transitory computer-readable media to implement embodiments of the present disclosure. In some embodiments, hard-wired circuitry may be used in place of or in combination with software instructions. Thus, embodiments of the present disclosure are not limited to any specific combination of hardware circuitry, firmware, and/or software.

The term “computer-readable medium” as used herein refers to any medium that stores instructions which may be provided to a processor for execution. Such a medium may take many forms, including but not limited to, non-volatile and volatile media. Common forms of non-transitory computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, a PROM, and EPROM, a FLASH-EPROM, any other memory chip or cartridge, or any other medium on which instructions which can be executed by a processor are stored. The computer readable media does not include carrier waves and electronic signals passing wirelessly or over wired connections.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Technical Field, such claims should not be limited by the language chosen under this heading to describe the so-called technical field. Further, a description of a technology in the “Background is not to be construed as an admission that technology is prior art to any invention(s) in this disclosure. Neither is the “Brief Summary” to be considered as a characterization of the invention(s) set forth in issued claims. Furthermore, any reference in this disclosure to “invention’ in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

1. A system, comprising:
a first database configured to store patient data including at least one of protected health information or personally identifiable information associated with a plurality of patients;
a second database separate from the first database, the second database configured to store customer relationship management data associated with a plurality of healthcare providers;
a server in communication with the first database and the second database, the server comprising one or more processors and a memory storing instructions that, when executed by the one or more processors, cause the server to:
receive, from a user computing device associated with a field representative, a request to access patient data associated with a patient case;
retrieve, from the first database, patient data associated with the patient case;
retrieve, from the second database, healthcare provider data associated with a healthcare provider linked to the patient case; and
transmit, to the user computing device, a user interface displaying the patient data and the healthcare provider data while maintaining the patient data in the first database separate from the second database.

2. The system of claim 1, wherein the instructions further cause the server to:
receive a fax document containing patient information;
perform optical character recognition on the fax document to extract text from the fax document;
identify a plurality of data fields within the extracted text; and
automatically populate a patient intake form with the plurality of data fields.

3. The system of claim 2, wherein the instructions further cause the server to:
receive a selection of a field in the patient intake form; and
display a visual indication on the fax document corresponding to a location from which data populating the selected field was extracted.

4. The system of claim 1, wherein the user interface includes a visual indicator adjacent to fields containing at least one of protected health information or personally identifiable information.

5. The system of claim 4, wherein data associated with fields having the visual indicator is encrypted during transmission.

6. The system of claim 1, wherein the instructions further cause the server to:
receive geographic location data associated with a plurality of patient cases;
display a map interface depicting locations associated with the plurality of patient cases;
receive a selection of a geographic area on the map interface; and
identify patient cases located within the selected geographic area for scheduling.

7. The system of claim 6, wherein the instructions further cause the server to:
generate a schedule grouping the identified patient cases based on proximity to each other.

8. The system of claim 1, wherein the patient data stored in the first database is not stored locally on the user computing device.

9. The system of claim 1, wherein the patient data stored in the first database is not stored in the second database.

10. The system of claim 1, wherein the instructions further cause the server to:
track a patient journey comprising a plurality of workflow stages including at least one of intake, enrollment, benefits verification, coverage verification, fulfillment, or adherence monitoring.

11. A method, comprising:
storing, in a first database, patient data including at least one of protected health information or personally identifiable information associated with a plurality of patients;
storing, in a second database separate from the first database, customer relationship management data associated with a plurality of healthcare providers;
receiving, by a server from a user computing device, a request to access data associated with a patient case;
retrieving, by the server from the first database, patient data associated with the patient case;
retrieving, by the server from the second database, healthcare provider data associated with a healthcare provider linked to the patient case; and
transmitting, by the server to the user computing device, a user interface displaying the patient data and the healthcare provider data while maintaining the patient data in the first database separate from the second database.

12. The method of claim 11, further comprising:
receiving a fax document containing patient information;
performing optical character recognition on the fax document to extract text from the fax document;
identifying a plurality of data fields within the extracted text; and
automatically populating a patient intake form with the plurality of data fields.

13. The method of claim 12, further comprising:
receiving a selection of a field in the patient intake form; and
displaying a visual indication on the fax document corresponding to a location from which data populating the selected field was extracted.

14. The method of claim 11, further comprising:
displaying a visual indicator adjacent to fields in the user interface containing at least one of protected health information or personally identifiable information.

15. The method of claim 11, further comprising:
receiving geographic location data associated with a plurality of patient cases;
displaying a map interface depicting locations associated with the plurality of patient cases;
receiving a selection of a geographic area on the map interface; and
identifying patient cases located within the selected geographic area for scheduling.

16. The method of claim 11, further comprising:
tracking a patient journey comprising a plurality of workflow stages including at least one of intake, enrollment, benefits verification, coverage verification, fulfillment, or adherence monitoring.

17. A non-transitory computer-readable medium storing instructions that, when executed by one or more processors, cause the one or more processors to:
maintain a first database storing patient data including at least one of protected health information or personally identifiable information associated with a plurality of patients;
maintain a second database separate from the first database, the second database storing customer relationship management data associated with a plurality of healthcare providers;
receive, from a user computing device associated with a field representative, a request to access data associated with a patient case;
retrieve, from the first database, patient data associated with the patient case;
retrieve, from the second database, healthcare provider data associated with a healthcare provider linked to the patient case; and
transmit, to the user computing device, a user interface displaying the patient data and the healthcare provider data while maintaining the patient data in the first database separate from the second database.

18. The non-transitory computer-readable medium of claim 17, wherein the instructions further cause the one or more processors to:
receive a fax document containing patient information;
perform optical character recognition on the fax document to extract text from the fax document; and
automatically populate a patient intake form with data extracted from the fax document.

19. The non-transitory computer-readable medium of claim 18, wherein the instructions further cause the one or more processors to:
receive a selection of a field in the patient intake form; and
display a visual indication on the fax document corresponding to a location from which data populating the selected field was extracted.

20. The non-transitory computer-readable medium of claim 17, wherein the instructions further cause the one or more processors to:
receive geographic location data associated with a plurality of patient cases;
display a map interface depicting locations associated with the plurality of patient cases; and
enable selection of patient cases within a geographic area for grouped scheduling.
